(19) United States
(12) Patent Application Publication
(10) Pub. No.: US 2025/0108107 A1
(43) Pub. Date: Apr. 3, 2025

(54) VACCINES AND RELATED METHODS

(71) Applicant: Flagship Pioneering Innovations VII, LLC, Cambridge, MA (US)

(72) Inventors: Daniel William Menon ACKER, Arlington, MA (US); John Edward HOLLAND, Somerville, MA (US); William Augustine DUNN, III, Roslindale, MA (US); Adam Spier DINGENS, Cambridge, MA (US); Julia Anna Kalter CHARTOVE, Cambridge, MA (US); Alexander Patrick GOLDSMITH, Winchester, MA (US); Ellen Lovisa Larsdotter AFZELIUS, Belmont, MA (US)

(21) Appl. No.: 18/903,675

(22) Filed: Oct. 1, 2024

Related U.S. Application Data
(63) Continuation of application No. PCT/US23/17945, filed on Apr. 7, 2023.
(60) Provisional application No. 63/362,710, filed on Apr. 8, 2022.
(60) Provisional application No. 63/476,310, filed on Dec. 20, 2022.

(51) Int. Cl. A61K 39/215; A61K 9/127; A61K 9/51; A61K 39/00; B82Y 5/00; C07K 14/005; C12N 7/00

(52) U.S. Cl. CPC A61K 39/215; A61K 9/1272; A61K 9/5123; C07K 14/005; C12N 7/00; A61K 2039/53; A61K 2039/55555; B82Y 5/00; C12N 2770/20022; C12N 2770/20034

(57) ABSTRACT

Provided herein are SARS-CoV-2 spike proteins and polypeptides (e.g., SARS-CoV-2 spike proteins and polypeptide immunogens (and immunogenic fragments and immunogenic variants thereof)) comprising at least one set of amino acid substitutions, and nucleic acid molecules encoding the same. Further provided herein are compositions (e.g., pharmaceutical compositions) and vaccines comprising the same for use in e.g., the prevention, treatment, and/or amelioration of a SARS-CoV-2 infection.

### 1. RELATED APPLICATIONS

This application claims priority to U.S. Ser. No. 63/362,710, filed Apr. 8, 2022 and U.S. Ser. No. 63/476,310, filed Dec. 20, 2022, the entire contents of each of which is incorporated herein by reference.

### 2. FIELD

This disclosure relates to SARS-CoV-2 spike proteins and polypeptides (e.g., SARS-CoV-2 spike protein and polypeptide immunogens (and immunogenic fragments and/or immunogenic variants thereof)), that comprise at least one set of amino acid substitutions described herein and nucleic acid molecules encoding the same. The disclosure further relates to compositions comprising the same (e.g., vaccine compositions, pharmaceutical compositions) and methods of making and utilizing the same.

### 3. BACKGROUND

Coronaviruses are a family of enveloped, positive-sense, single stranded RNA viruses that infect a wide variety of mammalian and avian species. The viral genome is packaged into a capsid that is comprised of the viral nucleocapsid protein and surrounded by a lipid envelope. Embedded in the lipid envelope are several proteins, including, the membrane protein, the envelope small membrane protein, hemagglutinin-esterase, and the spike protein. Human coronaviruses typically cause respiratory illnesses, and include, e.g., severe acute respiratory syndrome coronavirus 2 (SARS-CoV-2), severe acute respiratory syndrome coronavirus 1 (SARS-CoV-1), and Middle East respiratory syndrome (MERS-CoV).

SARS-Cov-2 emerged in humans in 2019, spread rapidly, and led to an ongoing global pandemic. SARS-CoV-2 is the cause of the coronavirus disease 2019 (COVID-19). COVID-19 has caused a continuing public health crisis, with millions of deaths and severe illness attributed to COVID-19 worldwide. Protection against COVID-19 is mediated in large part by an immune response directed against the SARS-CoV-2 spike protein, a main target of SARS-CoV-2 vaccines. The spike protein mediates binding and entry into host cells, through binding of the receptor binding domain (RBD) to the host cell receptor angiotensin-converting enzyme 2 (ACE2).

### 4. SUMMARY

Provided herein are, inter alia, SARS-CoV-2 spike proteins and polypeptides (e.g., SARS-CoV-2 spike protein or polypeptide immunogens (and immunogenic fragments and/or immunogenic variants thereof)), nucleic acid molecules encoding the same, compositions (e.g., vaccine compositions, pharmaceutical compositions) comprising the SARS-CoV-2 spike proteins and polypeptides (e.g., the SARS-CoV-2 spike protein or polypeptide immunogens (and immunogenic fragments and/or immunogenic variants thereof)) or nucleic acid molecules encoding the same, methods of manufacturing, and methods of utilizing the same including, e.g., methods of preventing, ameliorating, or treating a SARS-CoV-2 infection, methods of vaccination against a SARS-CoV-2 infection, etc.

In one aspect, provided herein are, inter alia, nucleic acid molecules comprising a coding region encoding a SARS-CoV-2 spike protein (e.g., a SARS-CoV-2 spike protein immunogen (or an immunogenic fragment and/or immunogenic variant thereof)), wherein the amino acid sequence of the SARS-CoV-2 spike protein (e.g., the SARS-CoV-2 spike protein immunogen (or immunogenic fragment and/or immunogenic variant thereof)) comprises at least one set of amino acid substitutions set forth in Table 2.

In some embodiments, the amino acid sequence of the encoded SARS-CoV-2 spike protein (e.g., the SARS-CoV-2 spike protein immunogen (or immunogenic fragment and/or immunogenic variant thereof)) comprises or consists of the SARS-CoV-2 spike protein receptor binding domain (RBD).

In some embodiments, the amino acid sequence of the encoded SARS-CoV-2 spike protein (e.g., the SARS-CoV-2 spike protein immunogen (or immunogenic fragment and/or immunogenic variant thereof)) comprises or consists of a full-length SARS-CoV-2 spike protein.

In some embodiments, the amino acid sequence of the encoded SARS-CoV-2 spike protein (e.g., the SARS-CoV-2 spike protein immunogen (or immunogenic fragment and/or immunogenic variant thereof)) comprises at least 10, 15, 20, 30, 40, 50, 60, 70, 80, 90, 100, 150, 200, 300, 400, 500, 600, 700, 800, 900, 1000, 1100, 1200, or 1300 amino acids.

In some embodiments, the amino acid sequence of the encoded SARS-CoV-2 spike protein (e.g., the SARS-CoV-2 spike protein immunogen (or immunogenic fragment and/or immunogenic variant thereof)) comprises from about 10-15, 10-20, 10-30, 10-40, 10-50, 10-60, 10-70, 10-80, 10-90, 10-100, 10-200, 10-300, 10-400, 10-500, 10-600, 10-700, 10-800, 10-900, 10-1000, 10-1100, 10-1200, or 10-1300 amino acids.

In some embodiments, other than the at least one set of amino acid substitutions set forth in Table 2, the amino acid sequence of the encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, 1200, or 1300 amino acids set forth in any one of SEQ ID NOS: 1-4.

In some embodiments, other than the at least one set of amino acid substitutions set forth in Table 2, the amino acid sequence of the encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-4.

In some embodiments, the amino acid sequence of the encoded SARS-CoV-2 spike protein (e.g., the SARS-CoV-2 spike protein immunogen (or immunogenic fragment and/or immunogenic variant thereof)) comprises a plurality of sets of amino acid substitutions set forth in Table 2.

In some embodiments, the amino acid sequence of the encoded SARS-CoV-2 spike protein (e.g., the SARS-CoV-2 spike protein immunogen (or immunogenic fragment and/or immunogenic variant thereof)) comprises at least 2, 3, 4, 5, or 6 or more sets of amino acid substitutions set forth in Table 2.

In some embodiments, the amino acid sequence of the encoded SARS-CoV-2 spike protein (e.g., the SARS-CoV-2 spike protein immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%) amino acid variations (e.g., substitutions, additions, deletions, etc.) that are not set forth in Table 2.

In some embodiments, the amino acid sequence of the encoded SARS-CoV-2 spike protein (e.g., the SARS-CoV-2 spike protein immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%) amino acid variations (e.g., substitutions, additions, deletions, etc.) that are not set forth in Table 2 relative to the amino acid sequence set forth in any one of SEQ ID NOS: 1-4.

In some embodiments, the encoded SARS-CoV-2 spike protein (e.g., the SARS-CoV-2 spike protein immunogen (or immunogenic fragment and/or immunogenic variant thereof)) is stabilized in a prefusion state.

In some embodiments, wherein the amino acid sequence of the encoded SARS-CoV-2 spike protein (e.g., the SARS-CoV-2 spike protein immunogen (or immunogenic fragment and/or immunogenic variant thereof)) comprises at least one amino acid variation relative to the amino acid sequence set forth in any one of SEQ ID NOS: 1-4, that stabilizes the SARS-CoV-2 spike protein (e.g., the SARS-CoV-2 spike protein immunogen (or immunogenic fragment and/or immunogenic variant thereof)) in a prefusion state.

In some embodiments, the amino acid sequence of the encoded SARS-CoV-2 spike protein (e.g., the SARS-CoV-2 spike protein immunogen (or immunogenic fragment and/or immunogenic variant thereof)) comprises a proline at amino acid position 986 and/or a proline at amino acid position 987, amino acid numbering relative to the amino acid positions set forth in SEQ ID NO: 4.

In some embodiments, the encoded SARS-CoV-2 spike protein (e.g., the SARS-CoV-2 spike protein immunogen (or immunogenic fragment and/or immunogenic variant thereof)) comprises one or more non-naturally N-glycosylation sites.

In some embodiments, the amino acid sequence of the encoded SARS-CoV-2 spike protein (e.g., the SARS-CoV-2 spike protein immunogen (or immunogenic fragment and/or immunogenic variant thereof)) comprises the addition of one or more N-glycosylation sites relative to the amino acid sequence set forth in any one of SEQ ID NOS: 1-4.

In some embodiments, the encoded SARS-CoV-2 spike protein is an immunogen (or an immunogenic fragment and/or immunogenic variant thereof).

In some embodiments, the nucleic acid molecule is RNA or DNA. In some embodiments, the RNA is messenger ribonucleic acid (mRNA).

In some embodiments, the nucleic acid molecule comprises at least one modified nucleotide. In some embodiments, the nucleic acid molecule comprises N1-methyl-pseudouridine, cytosine, adenine, and guanine.

In some embodiments, the nucleic acid molecule comprises a heterologous 5′-untranslated region (UTR), 3′-UTR, or both a 5′-UTR and 3′-UTR. In some embodiments, the nucleic acid molecule comprises a poly(A) sequence. In some embodiments, the nucleic acid molecule comprises a 5′cap structure. In some embodiments, the nucleotide sequence of the nucleic acid molecule is codon optimized.

In some embodiments, the nucleic acid molecule further encodes a heterologous polypeptide or protein.

In some embodiments, the nucleic acid molecule encodes a signal peptide. In some embodiments, the nucleic acid molecule encodes a homologous or heterologous signal peptide. In

In one aspect, provided herein are vectors comprising a nucleic acid described herein (e.g., a nucleic acid molecule comprising a coding region encoding a SARS-CoV-2 spike protein (e.g., a SARS-CoV-2 spike protein immunogen (or an immunogenic fragment and/or immunogenic variant thereof))). In some embodiments, the vector is a non-viral vector (e.g., a plasmid) or a viral vector.

In one aspect, provided herein are conjugates comprising a nucleic acid molecule described herein (e.g., a nucleic acid molecule comprising a coding region encoding a SARS-CoV-2 spike protein (e.g., a SARS-CoV-2 spike protein immunogen (or an immunogenic fragment and/or immunogenic variant thereof))) operably connected (e.g., directly or indirectly (e.g., via a linker)) to a heterologous moiety (e.g., a heterologous polypeptide or protein).

In one aspect, provided herein are compositions comprising at least one nucleic acid molecule described herein. In some embodiments, the composition comprises a plurality of nucleic acid molecules described herein (e.g., a plurality of nucleic acid molecules each comprising a coding region encoding a SARS-CoV-2 spike protein (e.g., a SARS-CoV-2 spike protein immunogen (or an immunogenic fragment and/or immunogenic variant thereof))), wherein the amino acid sequence of each of the encoded SARS-CoV-2 spike proteins (e.g., SARS-CoV-2 spike protein immunogens (or immunogenic fragments and/or immunogenic variants thereof)) encoded by of each of the plurality of nucleic acid molecules comprises a different set of amino acid substitutions set forth in Table 2. In some embodiments, the composition comprises a nucleic acid molecule comprising a coding region encoding SARS-CoV-2 spike proteins (e.g., SARS-CoV-2 spike protein immunogens (or immunogenic fragments and/or immunogenic variants thereof)) comprising an amino acid sequence that does not comprise a set of amino acid substitutions set forth in Table 2.

In one aspect, provided herein are SARS-CoV-2 spike proteins (e.g., SARS-CoV-2 spike protein immunogens (or immunogenic fragments and/or immunogenic variants thereof)) wherein the amino acid sequence of the SARS-CoV-2 spike protein (e.g., the SARS-CoV-2 spike protein immunogen (or immunogenic fragment and/or immunogenic variant thereof)) comprises at least one set of amino acid substitutions set forth in Table 2.

In some embodiments, the amino acid sequence of the SARS-CoV-2 spike protein (e.g., the SARS-CoV-2 spike protein immunogen (or immunogenic fragment and/or immunogenic variant thereof)) comprises or consists of the receptor binding domain (RBD) of a SARS-CoV-2 spike protein.

In some embodiments, the amino acid sequence of the SARS-CoV-2 spike protein (e.g., the SARS-CoV-2 spike protein immunogen (or immunogenic fragment and/or immunogenic variant thereof)) comprises or consists of a full-length SARS-CoV-2 spike protein.

In some embodiments, the amino acid sequence of the SARS-CoV-2 spike protein (e.g., the SARS-CoV-2 spike protein immunogen (or immunogenic fragment and/or immunogenic variant thereof)) comprises at least 10, 15, 20, 30, 40, 50, 60, 70, 80, 90, 100, 150, 200, 300, 400, 500, 600, 700, 800, 900, 1000, 1100, or 1200 amino acids.

In some embodiments, the amino acid sequence of the SARS-CoV-2 spike protein (e.g., the SARS-CoV-2 spike protein immunogen (or immunogenic fragment and/or immunogenic variant thereof)) comprises from about 10-15, 10-20, 10-30, 10-40, 10-50, 10-60, 10-70, 10-80, 10-90, 10-100, 10-200, 10-300, 10-400, 10-500, 10-600, 10-700, 10-800, 10-900, 10-1000, 10-1100, 10-1200, or 10-1300 amino acids.

In some embodiments, other than the at least one set of amino acid substitutions, the amino acid sequence of the SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-4.

In some embodiments, other than the at least one set of amino acid substitutions, the amino acid sequence of the SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-4.

In some embodiments, the amino acid sequence of the SARS-CoV-2 spike protein (e.g., the SARS-CoV-2 spike protein immunogen (or immunogenic fragment and/or immunogenic variant thereof)) comprises a plurality of amino acid substitutions set forth in Table 2.

In some embodiments, the amino acid sequence of the SARS-CoV-2 spike protein (e.g., the SARS-CoV-2 spike protein immunogen (or immunogenic fragment and/or immunogenic variant thereof)) comprises at least 2, 3, 4, 5, or 6 or more sets of amino acid substitutions set forth in Table 2.

In some embodiments, the amino acid sequence of the SARS-CoV-2 spike protein (e.g., the SARS-CoV-2 spike protein immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%) amino acid variations (e.g., substitutions, additions, deletions, etc.) that are not listed in Table 2.

In some embodiments, the amino acid sequence of the SARS-CoV-2 spike protein (e.g., the SARS-CoV-2 spike protein immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%) amino acid variations (e.g., substitutions, additions, deletions, etc.) that are not listed in Table 2 relative to the amino acid sequence set forth in any one of SEQ ID NOS: 1-4.

In some embodiments, the SARS-CoV-2 spike protein (e.g., the SARS-CoV-2 spike protein immunogen (or immunogenic fragment and/or immunogenic variant thereof)) is stabilized in a prefusion state.

In some embodiments, the amino acid sequence of the SARS-CoV-2 spike protein (e.g., the SARS-CoV-2 spike protein immunogen (or immunogenic fragment and/or immunogenic variant thereof)) comprises at least one amino acid variation relative to the amino acid sequence set forth in any one of SEQ ID NOS: 1-4 that stabilizes the SARS-CoV-2 spike protein (e.g., the SARS-CoV-2 spike protein immunogen (or immunogenic fragment and/or immunogenic variant thereof)) in a prefusion state.

In some embodiments, the amino acid sequence of the SARS-CoV-2 spike protein (e.g., the SARS-CoV-2 spike protein immunogen (or immunogenic fragment and/or immunogenic variant thereof)) comprises a proline at amino acid position 986 and/or a proline at amino acid position 987, amino acid numbering relative to the amino acid positions set forth in SEQ ID NO: 4.

In some embodiments, the SARS-CoV-2 spike protein (e.g., the SARS-CoV-2 spike protein immunogen (or immunogenic fragment and/or immunogenic variant thereof)) comprises one or more non-naturally N-glycosylation sites.

In some embodiments, the amino acid sequence of the SARS-CoV-2 spike protein (e.g., the SARS-CoV-2 spike protein immunogen (or immunogenic fragment and/or immunogenic variant thereof)) comprises the addition of one or more N-glycosylation sites relative to the amino acid sequence set forth in any one of SEQ ID NOS: 1-4.

In some embodiments, the amino acid sequence of the SARS-CoV-2 spike protein (e.g., the SARS-CoV-2 spike protein immunogen (or immunogenic fragment and/or immunogenic variant thereof)) comprises an inactive furin cleavage site.

In some embodiments, amino acid sequence of the SARS-CoV-2 spike protein (e.g., the SARS-CoV-2 spike protein immunogen (or immunogenic fragment and/or immunogenic variant thereof)) comprises at least one amino acid variation in the furin cleavage site that inactivates the furin cleavage site.

In some embodiments, the SARS-CoV-2 spike protein (e.g., the SARS-CoV-2 spike protein immunogen (or immunogenic fragment and/or immunogenic variant thereof)) further comprises a heterologous protein.

In some embodiments, the SARS-CoV-2 spike protein (e.g., the SARS-CoV-2 spike protein immunogen (or immunogenic fragment and/or immunogenic variant thereof)) comprises a signal peptide. In some embodiments, the SARS-CoV-2 spike protein (e.g., the SARS-CoV-2 spike protein immunogen (or immunogenic fragment and/or immunogenic variant thereof)) comprises a homologous or heterologous signal peptide. In some embodiments, the SARS-CoV-2 spike protein (e.g., the SARS-CoV-2 spike protein immunogen (or immunogenic fragment and/or immunogenic variant thereof)) does not comprise a signal peptide.

In some embodiments, the SARS-CoV-2 spike protein is an immunogen (or an immunogenic fragment and/or immunogenic variant thereof).

In one aspect, provided herein are compositions comprising at least one SARS-CoV-2 spike protein (e.g., at least one SARS-CoV-2 spike protein immunogen (or immunogenic fragment and/or immunogenic variant thereof)) described herein (e.g., a SARS-CoV-2 spike protein (e.g., a SARS-CoV-2 spike protein immunogen (or immunogenic fragment and/or immunogenic variant thereof) that comprises at least one set of amino acid substitutions set forth in Table 2). In some embodiments, the composition comprises a plurality of SARS-CoV-2 spike proteins (e.g., a plurality of SARS-CoV-2 spike protein immunogens (or immunogenic fragments and/or immunogenic variants thereof)) described herein, wherein the amino acid sequence of each of the plurality of SARS-CoV-2 spike proteins (e.g., the SARS-CoV-2 spike protein immunogens (or immunogenic fragments and/or immunogenic variants thereof)) comprises a different set of amino acid substitutions set forth in Table 2. In some embodiments, the composition comprises at least one SARS-CoV-2 spike protein (e.g., SARS-CoV-2 spike protein immunogen (or immunogenic fragment and/or immunogenic variant thereof)) comprising an amino acid sequence that does not comprise a set of amino acid substitutions set forth in Table 2.

In one aspect, provided herein are fusion proteins comprising a SARS-CoV-2 spike protein (e.g., the SARS-CoV-2 spike protein immunogen (or immunogenic fragment and/or immunogenic variant thereof)) described herein (e.g., a SARS-CoV-2 spike protein (e.g., a SARS-CoV-2 spike protein immunogen (or immunogenic fragment and/or immunogenic variant thereof) that comprises at least one set of amino acid substitutions set forth in Table 2) operably connected (e.g., directly or indirectly (e.g., via a linker)) to a heterologous polypeptide or protein.

In one aspect, provided herein are conjugates comprising a SARS-CoV-2 spike protein (e.g., the SARS-CoV-2 spike protein immunogen (or immunogenic fragment and/or immunogenic variant thereof)) described herein (e.g., a SARS-CoV-2 spike protein (e.g., a SARS-CoV-2 spike protein immunogen (or immunogenic fragment and/or immunogenic variant thereof) that comprises at least one set of amino acid substitutions set forth in Table 2) operably connected (e.g., directly or indirectly (e.g., via a linker)) to a heterologous moiety.

In one aspect, provide herein are SARS-CoV-2 spike proteins (e.g., a SARS-CoV-2 spike protein immunogen (or an immunogenic fragment and/or immunogenic variant thereof)) encoded by a nucleic acid molecule described herein.

In one aspect, provided herein are nucleic acid molecules encoding the SARS-CoV-2 spike protein (e.g., the SARS-CoV-2 spike protein immunogen (or immunogenic fragment and/or immunogenic variant thereof)) described herein.

In one aspect, provide herein are carriers comprising a nucleic acid molecule described herein, a vector described herein, a composition described herein, a SARS-CoV-2 spike protein described herein, a vaccine composition described herein, a conjugate described herein, a fusion protein described herein, or a pharmaceutical composition described herein. In some embodiments, the carrier is a lipid nanoparticle (LNP), liposome, lipoplex, or nanoliposome. In some embodiments, the carrier is an LNP. In some embodiments, the LNP comprises a cationic lipid, a neutral lipid, a cholesterol, and/or a PEG lipid. In some embodiments, the LNP has a mean particle size of between 80 nm and 160 nm.

In one aspect, described herein are vaccine compositions comprising a nucleic acid molecule described herein, a vector described herein, a composition described herein, a SARS-CoV-2 spike protein described herein, a vaccine composition described herein, a conjugate described herein, a fusion protein described herein, a carrier described herein, or a pharmaceutical composition described herein.

In some embodiments, the vaccine composition is a prime vaccine composition. In some embodiments, the vaccine composition is a boost vaccine composition. In some embodiments, the vaccine composition is a prime vaccine composition and a boost vaccine composition. In some embodiments, the vaccine composition can be utilized as a prime vaccine composition and/or a booster vaccine composition in a homologous or heterologous prime boost vaccine regimen. In some embodiments, the vaccine composition further comprises an adjuvant.

In one aspect, provided herein are vaccine compositions comprising a messenger ribonucleic acid (mRNA) encoding a SARS-CoV-2 spike protein (e.g., a SARS-CoV-2 spike protein immunogen (or an immunogenic fragment and/or immunogenic variant thereof)) that comprises at least one amino acid substitution set forth in Table 2, formulated in a lipid nanoparticle, the vaccine composition having the following characteristics: (a) the LNPs comprise a cationic lipid, a neutral lipid, a cholesterol, and a PEG lipid, (b) the LNPs have a mean particle size of between 80 nm and 160 nm, and (c) the mRNA comprises: (i) a 5′-cap structure; (ii) a 5′-UTR; (iii) N1-methyl-pseudouridine, cytosine, adenine, and guanine; (iv) a 3′-UTR; and (v) a poly-A region.

In some embodiments, the vaccine composition is a prime vaccine composition. In some embodiments, the vaccine composition is a boost vaccine composition. In some embodiments, the vaccine composition is a prime vaccine composition and a boost vaccine composition. In some embodiments, the vaccine composition can be utilized as a prime vaccine composition and/or a booster vaccine composition in a homologous or heterologous prime boost vaccine regimen. In some embodiments, the vaccine composition further comprises an adjuvant.

In one aspect, provided herein are pharmaceutical compositions compositions comprising a nucleic acid molecule described herein, a vector described herein, a composition described herein, a SARS-CoV-2 spike protein described herein, a vaccine composition described herein, a conjugate described herein, a fusion protein described herein, a carrier described herein, or a vaccine composition described herein, and a pharmaceutically acceptable excipient. In some embodiments, the pharmaceutical composition further comprises an adjuvant.

In one aspect, provide herein are pharmaceutical compositions comprising a messenger ribonucleic acid (mRNA) encoding a SARS-CoV-2 spike protein (e.g., a SARS-CoV-2 spike protein immunogen (or an immunogenic fragment and/or immunogenic variant thereof)) that comprises at least one amino acid substitution set forth in Table 2, formulated in a lipid nanoparticle, the pharmaceutical composition having the following characteristics: (a) the LNPs comprise a cationic lipid, a neutral lipid, a cholesterol, and a PEG lipid, (b) the LNPs have a mean particle size of between 80 nm and 160 nm, and (c) the mRNA comprises: (i) a 5′-cap structure; (ii) a 5′-UTR; (iii) N1-methyl-pseudouridine, cytosine, adenine, and guanine; (iv) a 3′-UTR; and (v) a poly-A region. In some embodiments, the pharmaceutical composition further comprises an adjuvant.

In one aspect, provided herein are kits comprising a nucleic acid molecule described herein, a vector described herein, a composition described herein, a SARS-CoV-2 spike protein described herein, a vaccine composition described herein, a conjugate described herein, a fusion protein described herein, a carrier described herein, a vaccine composition described herein, or a pharmaceutical composition described herein.

In some embodiments, the kit comprises instructions for use of the nucleic acid molecule, vector, protein (or immunogenic fragment or immunogenic variant thereof), conjugate, fusion protein, carrier, composition, vaccine composition, or pharmaceutical composition.

In one aspect, provided herein are methods of delivering a nucleic acid molecule, vector, protein (or immunogenic fragment or immunogenic variant thereof), conjugate, fusion protein, carrier, composition, vaccine composition, or pharmaceutical composition to a subject in need thereof, the method comprising administering to the subject a nucleic acid molecule described herein, a vector described herein, a composition described herein, a SARS-CoV-2 spike protein described herein, a vaccine composition described herein, a conjugate described herein, a fusion protein described herein, a carrier described herein, a vaccine composition described herein, or a pharmaceutical composition described herein, to thereby deliver the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition to the subject.

In some embodiments, the subject is a human.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

In one aspect, provided herein are methods of inducing or enhancing an immune response in a subject in need thereof, the method comprising administering to the subject a nucleic acid molecule described herein, a vector described herein, a composition described herein, a SARS-CoV-2 spike protein described herein, a vaccine composition described herein, a conjugate described herein, a fusion protein described herein, a carrier described herein, a vaccine composition described herein, or a pharmaceutical composition described herein, to thereby induce or enhance an immune response the subject.

In some embodiments, the subject is a human.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

In one aspect, provide herein are methods of preventing, ameliorating, or treating a SARS-CoV-2 infection in a subject in need thereof, the method comprising administering to the subject a nucleic acid molecule described herein, a vector described herein, a composition described herein, a SARS-CoV-2 spike protein described herein, a vaccine composition described herein, a conjugate described herein, a fusion protein described herein, a carrier described herein, a vaccine composition described herein, or a pharmaceutical composition described herein, to thereby prevent, ameliorate, or treat the SARS-CoV-2 infection the subject.

In some embodiments, the subject is a human.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

In one aspect, provided herein are methods of vaccinating a subject against SARS-CoV-2, the method comprising administering to the subject a nucleic acid molecule described herein, a vector described herein, a composition described herein, a SARS-CoV-2 spike protein described herein, a vaccine composition described herein, a conjugate described herein, a fusion protein described herein, a carrier described herein, a vaccine composition described herein, or a pharmaceutical composition described herein, to thereby vaccinate the subject against SARS-CoV-2.

In some embodiments, the subject is a human.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

In one aspect, provided herein are methods of vaccinating a subject against SARS-CoV-2, the method comprising administering to the subject (a) an mRNA molecule (e.g., an mRNA molecule described herein) encoding the SARS-CoV-2 spike protein (e.g., a SARS-CoV-2 spike protein or polypeptide immunogen (or immunogenic fragment and/or immunogenic variant thereof)) described herein (or a conjugate or fusion protein thereof), (b) a vector comprising the mRNA molecule, (c) a carrier comprising the mRNA molecule or the vector, (d) a vaccine composition comprising the mRNA molecule, the vector, or the carrier, or (e) a pharmaceutical composition comprising the mRNA molecule, the vector, the carrier, or the vaccine composition, to thereby vaccinate the subject against SARS-CoV-2, to thereby vaccinate the subject against SARS-CoV-2.

In some embodiments, the subject is a human.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

In one aspect, provided herein are methods of vaccinating a subject against SARS-CoV-2, the method comprising administering to the subject a vaccine composition described herein or a pharmaceutical composition described herein, to thereby vaccinate the subject against SARS-CoV

In some embodiments, the subject is a human.

In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen. In some embodiments, the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.

### 5. DETAILED DESCRIPTION

SARS-CoV-2 continues to evolve into new variants comprising a variety of amino acid variations, e.g., substitutions, deletions, additions. Many of the variations are found in the RBD of the spike protein, which is vital for entry of the SARS-Cov-2 virus into host cells. As most of the SARS-CoV-2 vaccines and current antibody therapies target the RBD of the spike protein, this creates the potential for the evolution of SARS-CoV-2 variants that evade vaccine induced immunity, infection induced immunity, or current antibody therapies. The inventors have, inter alia, identified sequence variations in the SARS-CoV-2 spike protein (e.g., in the RBD) that e.g., counter SARS-CoV-2 resistance to vaccine induced immunity and/or are potential SARS-CoV-2 variants. Accordingly, novel SARS-CoV-2 spike proteins and polypeptides (e.g., SARS-CoV-2 spike protein or polypeptide immunogens (and immunogenic fragments and/or immunogenic variants thereof)) (or nucleic acid molecules, e.g., mRNAs, encoding such SARS-CoV-2 spike proteins and polypeptides (e.g., SARS-CoV-2 spike protein or polypeptide immunogens (and immunogenic fragments and/or immunogenic variants thereof))) comprising at least one such sequence variation are good candidates for variant-proof or variant-resistant vaccines against COVID-19. A such, the current disclosure provides, inter alia, novel SARS-CoV-2 spike proteins (e.g., SARS-CoV-2 spike protein or polypeptide immunogens) for use in, inter alia, pharmaceutical compositions and vaccines to induce a desired immune response against one or more variants of SARS-CoV-2.

### 5.1 Definitions

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed.

In this application, the use of the singular includes the plural unless specifically stated otherwise. For example, as used in the specification and the appended claims, the singular forms “a,” “an,” and “the” include plural referents unless the context clearly dictates otherwise. Furthermore, use of the term “including” as well as other forms, such as “include,” “includes,” and “included,” is not limiting.

It is understood that wherever aspects are described herein with the language “comprising,” otherwise analogous aspects described in terms of “consisting of” and “consisting essentially of” are also provided.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As described herein, any concentration range, percentage range, ratio range or integer range is to be understood to include the value of any integer within the recited range and, when appropriate, fractions thereof (such as one tenth and one hundredth of an integer), unless otherwise indicated.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. When particular values or compositions are provided in the application and claims, unless otherwise stated, the meaning of “about” or “comprising essentially of” should be assumed to be within an acceptable error range for that particular value or composition.

Where proteins and/or polypeptides are described herein, it is understood that nucleic acid molecules (e.g., RNA (e.g., mRNA) or DNA nucleic acid molecules) encoding the protein or polypeptide are also provided herein.

Where proteins, polypeptides, nucleic acid molecules, vectors, carriers, etc. are described herein, it is understood that isolated forms of the proteins, polypeptides, nucleic acid molecules, vectors, carriers, etc. are also provided herein.

Where proteins, polypeptides, nucleic acid molecules, vectors, carriers, etc. are described herein, it is understood that recombinant forms of the proteins, polypeptides, nucleic acid molecules, vectors, carriers, etc. are also provided herein.

Where polypeptides or sets of polypeptides are described herein, it is understood that proteins comprising the polypeptides or sets of polypeptides folded into their three-dimensional structure (i.e., tertiary or quaternary structure) are also provided herein and vice versa (i.e., where proteins are described herein polypeptides comprising the amino acid sequence of the protein are also provided herein).

As used herein, the term “adjuvant” refers to a substance that causes stimulation of the immune system of a subject when administered to the subject.

As used herein, the term “administering” refers to the physical introduction of an agent (e.g., a therapeutic agent, a vaccine) (or a precursor of the agent that is metabolized or altered (e.g., translation of a nucleic acid molecule) within the body of the subject to produce the agent in vivo) to a subject, using any of the various methods and delivery systems known to those skilled in the art. Administering can also be performed, for example, once, a plurality of times, and/or over one or more extended periods.

As used herein, the term “agent” is used generically to describe any macro or micro molecule. Exemplary moieties include, but are not limited to polypeptides, proteins, peptides, nucleic acid molecules (e.g., DNA, RNA), small molecules, carbohydrates, lipids, synthetic polymers (e.g., polymers of PEG).

As used herein, the term “derived from,” with reference to a nucleic acid molecule refers to a nucleic acid molecule that has at least 70% sequence identity to a reference nucleic acid molecule (e.g., a naturally occurring nucleic acid molecule) or a fragment thereof. The term “derived from,” with reference to a polypeptide or protein refers to a polypeptide or protein that comprises an amino acid sequence that has at least 70% sequence identity to the amino acid sequence of a reference polypeptide or protein (e.g., a naturally occurring polypeptide or protein). The term “derived from” as used herein does not denote any specific process or method for obtaining the nucleic acid molecule, polypeptide, or protein. For example, the nucleic acid molecule, polypeptide, or protein can be recombinant produced or chemically synthesized.

As used herein, the term “disease” refers to any abnormal condition that impairs physiological function. The term is used broadly to encompass any disorder, illness, abnormality, pathology, sickness, condition, or syndrome in which physiological function is impaired, irrespective of the nature of the etiology. The term disease includes infection (e.g., a viral (e.g., a SARS-Cov-2 infection), bacterial, fungal, protozoal infection).

The terms “DNA” and “polydeoxyribonucleotide” are used interchangeably herein and refer to macromolecules that include multiple deoxyribonucleotides that are polymerized via phosphodiester bonds. Deoxyribonucleotides are nucleotides in which the sugar is deoxyribose.

As used herein, the term “Fe region” refers to the C-terminal region of an immunoglobulin (Ig) heavy chain that comprises from N- to C-terminus at least a CH2 region operably connected to a CH3 region. In some embodiments, the Fc region comprises an Ig hinge region or at least a portion of an Ig hinge region operably connected to the N-terminus of the CH2 region. In some embodiments, the Fc region is engineered relative to a reference Fc region. Additional examples of proteins with engineered Fc regions can be found in Saunders 2019 (K. O. Saunders, “Conceptual Approaches to Modulating Antibody Effector Functions and Circulation Half-Life,” 2019, Frontiers in Immunology, V. 10, Art. 1296, pp. 1-20, the entire contents of which is incorporated by reference herein for all purposes).

As used herein, the term “full-length” with reference to a SARS-CoV-2 spike protein refers to a SARS-CoV-2 spike protein, wherein the amino acid sequence of the SARS-CoV-2 spike protein comprises substantially the same number of amino acids as a reference SARS-CoV-2 spike protein (e.g., a reference naturally occurring SARS-CoV-2 spike protein).

As used herein, the term “fuse” and grammatical equivalents thereof refer to the operable connection of at least a first polypeptide or protein to a second polypeptide or protein, wherein the first and second polypeptides or proteins are not naturally found operably connected together. For example, the first and second polypeptides or proteins are derived from different proteins. The term fuse encompasses both a direct connection of the at least two polypeptides or proteins through a peptide bond, and the indirect connection through a linker (e.g., a peptide linker).

As used herein, the term “fusion protein” and grammatical equivalents thereof refers to a protein that comprises at least one polypeptide operably connected to another polypeptide, wherein the first and second polypeptides are different and not naturally found operably connected together. For example, the first and second polypeptides of the fusion protein are each derived from different proteins. The at least two polypeptides of the fusion protein can be directly operably connected through a peptide bond; or can be indirectly operably connected through a linker (e.g., a peptide linker). Therefore, for example, the term fusion polypeptide encompasses embodiments, wherein Polypeptide A is directly operably connected to Polypeptide B through a peptide bond (Polypeptide A-Polypeptide B), and embodiments, wherein Polypeptide A is operably connected to Polypeptide B through a peptide linker (Polypeptide A-peptide linker-Polypeptide B).

As used herein, the term “half-life extension moiety” refers to a moiety (e.g., small molecule, polypeptide, polynucleotide, carbohydrate, lipid, synthetic polymer (e.g., polymers of PEG), etc.) that when conjugated or otherwise operably connected (e.g., fused) to another moiety (the subject moiety) (e.g., a protein), increases the half-life of the subject moiety (e.g., protein) in vivo when administered to a subject (e.g., a human subject). The pharmacokinetic properties of the subject moiety (e.g., protein) can be evaluated utilizing in vivo models known in the art.

As used herein, the term “half-life extension protein or “half-life extension polypeptide” refers to a polypeptide or protein that when operably connected to another moiety (e.g., a subject moiety) (e.g., a protein), increases the half-life of the subject moiety (e.g., the subject protein) in vivo when administered to a subject (e.g., a human subject). The pharmacokinetic properties of the protein can be evaluated utilizing in vivo models known in the art.

As used herein, the term “heterologous”, when used to describe a first element in reference to a second element means that the first element and second element do not exist in nature disposed as described. For example, a polypeptide comprising a “heterologous moiety” means a polypeptide that is joined to a moiety (e.g., small molecule, polypeptide, nucleic acid molecule, carbohydrate, lipid, synthetic polymer (e.g., polymers of PEG), etc.) that is not joined to the polypeptide in nature.

As used, herein the term “heterologous signal peptide” refers to a signal peptide that is not operably connected to a subject polypeptide or protein in nature. For example, in reference to a polypeptide comprising a signal peptide from human IL-2 operably connected to human IL-12, the human IL-2 signal peptide would constitute a heterologous signal peptide.

As used herein, the term “homologous signal peptide” refers to a signal peptide that is operably connected to a subject polypeptide or protein in nature. For example, in reference to a polypeptide comprising a signal peptide from human IL-2 operably connected to human IL-2, the human IL-2 signal peptide would constitute a homologous signal peptide.

As used herein, the term “prime boost” refers to a vaccine regimen comprising at least an initial vaccine dose and one or more subsequent vaccine doses. The initial vaccine dose comprises the prime vaccine composition and the one or more subsequent vaccine doses are referred to as boost (or booster) vaccine compositions. Prime boost vaccine regimens can comprise more than one booster (e.g., 2, 3, 4, 5, 6, or more, etc.).

As used herein, the term “homologous prime boost” refers to a prime boost vaccine regimen wherein the prime vaccine composition and the boost (or booster) vaccine composition are the same.

As used herein, the term “heterologous prime boost” refers to a prime boost vaccine regimen wherein the prime vaccine composition and the boost (or booster) vaccine composition are different (e.g., the immunogen is different, the form of the immunogen is different (e.g., a nucleic acid (e.g., mRNA) molecule-based vaccine versus a protein-based vaccine), the immunogen is expressed from a different vector (e.g., plasmid, viral vector), the method of delivering the immunogen to the subject is different, etc.).

As used herein, the term “immunogen” refers to a substance that is capable of inducing an immune response (e.g., an adaptive immune response) in a subject (e.g., a human subject).

As used herein, the term “immunogenic fragment” refers to a fragment of a reference polypeptide or protein that retains an immunogen.

As used herein, the term “immunogenic variant” refers to a variant of a reference polypeptide or protein that retains an immunogen. In some embodiments, the polypeptide or protein comprises at least one but no more than 25%, (e.g., no more than 20%, no more than 15%, no more than 12%, no more than 10%, no more than 8%) amino acid variation (e.g., substitutions, deletions, additions) compared to the amino acid sequence of a reference polypeptide or protein.

As used herein, the term “in combination with” means that two (or more) different agents or treatments are administered to a subject as part of a defined treatment regimen for a particular disease. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen (e.g., a prime-boost vaccine regimen). In some embodiments, administration of two or more agents or treatments in combination is such that the reduction in a symptom, or other parameter related to the disease is greater than what would be observed with one agent or treatment delivered alone or in the absence of the other. The effect of the two treatments can be partially additive, wholly additive, or greater than additive (e.g., synergistic). Sequential or substantially simultaneous administration of each agent can be effected by any appropriate route including, but not limited to, oral routes, intravenous routes, intramuscular routes, and direct absorption through mucous membrane tissues. The agents can be administered by the same route or by different routes.

As used herein, the term “isolated” with reference to an agent (e.g., a polypeptide, protein, or nucleic acid molecule) refers to the agent (e.g., the polypeptide, protein, or nucleic acid molecule) that is substantially free of other cellular components with which it is associated in the natural state.

As used herein, the term “long COVID” is commonly used to refer to signs and symptoms that continue or develop after acute COVID-19. Long COVID is also referred to in the art as persistent post-Covid syndrome (PPCS), post-acute sequelae of COVID-19 (PASC), long haul COVID, and chronic COVID. The term long COVID encompasses any clinically acceptable definition.

As used herein, the term “modification” in reference to a nucleic acid sequence refers to a nucleic acid molecule that comprises at least one nucleotide comprising a chemical modification, e.g., a modified sugar moiety, a modified nucleobase, and/or a modified internucleotide linkage, or any combination thereof. Exemplary nucleotide modifications are provided herein, see, e.g., § 5.3 (e.g., § 5.3.2). In certain embodiments of the instant disclosure, inclusion of a deoxynucleotide—which is acknowledged as a naturally occurring form of nucleotide—if present within an RNA molecule (e.g., an mRNA molecule) is considered to constitute a modified nucleotide.

The terms “nucleic acid molecule” and “polynucleotide” are used interchangeably herein and refer to a polymer of DNA or RNA. The nucleic acid molecule can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified nucleic acid molecule. Nucleic acid molecules include, but are not limited to, all nucleic acid molecules which are obtained by any means available in the art, including, without limitation, recombinant means, e.g., the cloning of nucleic acid molecules from a recombinant library or a cell genome, using ordinary cloning technology and polymerase chain reaction, and the like, and by synthetic means. The skilled artisan will appreciate that, except where otherwise noted, nucleic acid sequences set forth in the instant application will recite thymidine (T) in a representative DNA sequence but where the sequence represents RNA (e.g., mRNA), the thymidines (Ts) would be substituted for uracils (Us). Thus, any of the RNA nucleic acid molecules encoded by a DNA identified by a particular sequence identification number may also comprise the corresponding RNA (e.g., mRNA) sequence encoded by the DNA, where each thymidine (T) of the DNA sequence is substituted with uracil (U).

As used herein, the term “operably connected” refers to the linkage of two moieties in a functional relationship. For example, a polypeptide is operably connected to another polypeptide when they are linked (either directly or indirectly via a peptide linker) in frame such that both polypeptides are functional (e.g., a fusion protein described herein). Or for example, a transcription regulatory polynucleotide e.g., a promoter, enhancer, or other expression control element is operably connected to a polynucleotide that encodes a protein if it affects the transcription of the polynucleotide that encodes the protein. The term “operably connected” can also refer to the conjugation of a moiety to e.g., a polynucleotide or polypeptide (e.g., the conjugation of a PEG polymer to a protein).

As used herein, the term “peptide” refers to a polymer of at least two amino acids linked by peptide bonds. The term “peptide” does not limit the length of the polymer chain of amino acids. It is common in the art to refer to shorter polymers of amino acids (e.g., approximately 2-50 amino acids) as peptides; and to refer to longer polymers of amino acids (e.g., approximately over 50 amino acids) as polypeptides. However, the terms “peptide” and “polypeptide” are used interchangeably herein.

The determination of “percent identity” between two sequences (e.g., peptide or protein (amino acid sequences) or nucleic acid sequences)) can be accomplished using a mathematical algorithm. A specific, non-limiting example of a mathematical algorithm utilized for the comparison of two sequences is the algorithm of Karlin S & Altschul S F (1990) PNAS 87: 2264-2268, modified as in Karlin S & Altschul S F (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the term “pharmaceutical composition” refers to a composition that is suitable for administration to an animal (e.g., a human subject) and comprises a therapeutic agent and a pharmaceutically acceptable carrier or diluent. A “pharmaceutically acceptable carrier or diluent” means a substance for use in contact with the tissues of human beings and/or non-human animals without excessive toxicity, irritation, allergic response, or other problem or complication, commensurate with a reasonable therapeutic benefit/risk ratio.

As used herein, the term “plurality” means 2 or more (e.g., 3 or more, 4 or more, 5 or more, 6 or more, 7 or more, 9 or more, or 10 or more).

As used herein, the term “poly(A) sequence” refers to a sequence of adenosine nucleotides, typically located at the 3′-end of a linear RNA (or in a circular RNA), of up to about 1000 adenosine nucleotides. In some embodiments, the poly(A) sequence is essentially homopolymeric, e.g., a poly(A) sequence of e.g., 100 adenosine nucleotides has essentially the length of 100 nucleotides. In other embodiments, the poly(A) sequence may be interrupted by at least one nucleotide different from an adenosine nucleotide, e.g., a poly(A) sequence of e.g., 100 adenosine nucleotides may have a length of more than 100 nucleotides (comprising 100 adenosine nucleotides and in addition said at least one nucleotide—or a stretch of nucleotides—different from an adenosine nucleotide). It has to be understood that “poly(A) sequence” as defined herein typically relates to RNA—however in the context of the invention, the term likewise relates to corresponding sequences in a DNA molecule (e.g., a “poly(T) sequence”).

As used herein, the term, “prime-boost” with reference to a vaccine regimen refers to a vaccine regimen comprising a first administration of a first immunogen to a subject (the vaccine prime) and sometime thereafter administration of a vaccine booster (e.g., a second immunogen). In some embodiments, the vaccine booster comprises a second immunogen. As described herein, first immunogen of the vaccine prime and the second immunogen of the vaccine booster can be the same or different, administered via the same or different routes, etc.

A “prophylactic” treatment is a treatment administered to a subject who does not exhibit signs of a disease or exhibits only early signs for the purpose of decreasing the risk of developing pathology.

As used herein, the term “protein” refers to a one or more peptides folded into its three-dimensional structure.

As used herein, the term “receptor binding domain” or “RBD” in reference to a SARS-CoV-2 spike protein refers to the minimal amino acid sequence required for the SARS-CoV-2 spike protein to bind ACE2. The amino acid sequence of an exemplary reference SARS-CoV-2 spike protein RBD is set forth in SEQ ID NO: 1.

The terms “RNA” and “polyribonucleotide” are used interchangeably herein and refer to macromolecules that include multiple ribonucleotides that are polymerized via phosphodiester bonds. Ribonucleotides are nucleotides in which the sugar is ribose. RNA may contain modified nucleotides; and contain natural, non-natural, or altered internucleotide linkages, such as a phosphoroamidate linkage or a phosphorothioate linkage, instead of the phosphodiester found between the nucleotides of an unmodified nucleic acid molecule.

As used herein, the term “SARS-CoV-2 spike protein” refers to the SARS-CoV-2 protein that mediates binding to the host cell receptor angiotensin-converting enzyme 2 (ACE2). The amino acid sequence of a first exemplary reference SARS-CoV-2 spike protein is set forth in SEQ ID NO: 2. The term SARS-CoV-2 spike protein includes naturally occurring and engineered variants. The SARS-CoV-2 spike proteins and polypeptides described herein (e.g., the SARS-CoV-2 spike proteins and polypeptides) include fragments and variants thereof (e.g., immunogenic fragments and/or immunogenic variants thereof).

As used herein, the term “set” with reference to amino acid variation(s) (e.g., substitution(s)) does not require more than one amino acid variation (e.g., substitution). For example, Table 2 herein describes “sets” of amino acid substitutions, some sets have only one amino acid substitution and some sets have more than one amino acid substitution.

As used herein, the term “signal peptide” or “signal sequence” refers to a sequence (e.g., an amino acid sequence) that can direct the transport or localization of a protein to a certain organelle, cell compartment, or extracellular export. The term encompasses both the signal peptide (the amino acid sequence of the signal peptide) and the nucleic acid sequence encoding the signal peptide. Thus, references to a signal peptide in the context of a nucleic acid molecule refers to the nucleic acid sequence encoding the signal peptide.

As used herein, the term “subject” includes any animal, such as a human or other animal. In embodiments, the subject is a vertebrate animal (e.g., mammal, bird, fish, reptile, or amphibian). In embodiments, the subject is a human. In embodiments, the method subject is a non-human mammal. In embodiments, the subject is a non-human mammal is such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit). In embodiments, the subject is a bird, such as a member of the avian taxa Galliformes (e.g., chickens, turkeys, pheasants, quail), Anseriformes (e.g., ducks, geese), Paleaognathae (e.g., ostriches, emus), Columbiformes (e.g., pigeons, doves), or Psittaciformes (e.g., parrots).

A “therapeutically effective amount” of an agent (e.g., a therapeutic agent, a vaccine) refers to any amount of the agent (e.g., the therapeutic agent, the vaccine) that, when used alone or in combination with another agent (e.g., a therapeutic agent, a vaccine), protects a subject against the onset of a disease (e.g., an infection), ameliorates the severity of a disease (e.g., an infection), and/or promotes disease (e.g., infection) regression evidenced by a decrease in severity of disease (e.g., infection) symptoms, an increase in frequency and duration of disease (e.g., infection) symptom-free periods, or a prevention of impairment or disability due to the disease (e.g., infection) affliction. The ability of an agent (e.g., a therapeutic agent, a vaccine) to do any of the foregoing (or any combination thereof) can be evaluated using a variety of methods known to the skilled practitioner, such as in human subjects during clinical trials, in animal model systems predictive of efficacy in humans, or by assaying the activity of the agent in in vitro assays.

As used herein, the term “translatable RNA” refers to any RNA that encodes at least one polypeptide or protein and can be translated to produce the encoded polypeptide or protein in vitro, in vivo, in situ or ex vivo. A translatable RNA may be an mRNA or a circular RNA encoding a polypeptide or protein.

As used herein, the terms “treat,” treating,” “treatment,” and the like refer to reducing or ameliorating a disease (e.g., an infection) and/or symptom(s) associated therewith or obtaining a desired pharmacologic and/or physiologic effect. It will be appreciated that, although not precluded, treating a disease (e.g., an infection) does not require that the disease (e.g., an infection), or symptom(s) associated therewith be completely eliminated. In some embodiments, the effect is therapeutic, i.e., without limitation, the effect partially or completely reduces, diminishes, abrogates, abates, alleviates, decreases the intensity of, or cures a disease (e.g., an infection) and/or adverse symptom attributable to the disease (e.g., an infection). In some embodiments, the effect is preventative, i.e., the effect protects or prevents an occurrence or reoccurrence of a disease (e.g., an infection) or prevents severe disease (e.g., a severe infection, a severe disease associated with an infection).

As used herein, the term “variation” or “variant” with reference to a nucleic acid molecule, refers to a nucleic acid molecule that comprises at least one substitution, alteration, inversion, addition, or deletion of nucleotide compared to a reference nucleic acid molecule. As used herein, the term “variation” or “variant” with reference to a polypeptide or protein refers to a polypeptide or protein that comprises at least one substitution, alteration, inversion, addition, or deletion of an amino acid residue compared to a reference polypeptide or protein.

As used herein, the term “5′-untranslated region” or “5′-UTR” refers to a part of a nucleic acid molecule located 5′ (i.e., “upstream”) of a coding sequence and which is not translated into protein or polypeptide. Typically, a 5′-UTR starts with the transcriptional start site and ends before the start codon of the coding sequence. A 5′-UTR may comprise elements for controlling gene expression, also called regulatory elements. Such regulatory elements may be, e.g., ribosomal binding sites, miRNA binding sites etc. The 5′-UTR may be post-transcriptionally modified, e.g., by enzymatic or post-transcriptional addition of a 5′-cap structure.

As used herein the term “3′-untranslated region” or “3′-UTR” refers to a part of a nucleic acid molecule located 3′ (i.e., downstream) of a coding sequence and which is not translated into protein or polypeptide. A 3′-UTR may located between a coding sequence and an (optional) terminal poly(A) sequence of a nucleic acid sequence. A 3′-UTR may comprise elements for controlling gene expression, also called regulatory elements. Such regulatory elements may be, e.g., ribosomal binding sites, miRNA binding sites etc.

### 5.2 SARS-CoV-2 Spike Proteins (e.g., Immunogens)

The SARS-CoV-2 spike protein mediates viral entry into host cells. The spike protein comprises two functional subunits responsible for binding to the host cell receptor (S1 subunit) and fusion of the viral and cellular membranes (S2 subunit). The SARS-CoV-2 spike protein is cleaved at the boundary between the S1 and S2 subunits, which remain non-covalently associated in the prefusion conformation. The distal S1 subunit comprises the RBD and contributes to stabilization of the prefusion state of the membrane anchored S2 subunit that contains the fusion machinery. The RBD mediates binding to the host cell receptor ACE2. The spike protein is cleaved by host proteases at the so-called S2′ site located immediately upstream of the fusion peptide. This cleavage has been proposed to activate the protein for membrane fusion via extensive irreversible conformational changes. (See, e.g., Walls, Alexandra C et al. “Structure, Function, and Antigenicity of the SARS-CoV-2 Spike Glycoprotein.” Cell vol. 181, 2 (2020): 281-292.e6. doi:10.1016/j.cell.2020.02.058, the entire contents of which is incorporated herein by reference for all purposes).

The amino acid sequence of an exemplary reference SARS-CoV-2 spike protein is provided in SEQ ID NO: 2. The signal sequence is boxed (amino acids 1-13 of SEQ ID NO: 2), the S1 subunit is boldface (amino acids 14-682 of SEQ ID NO: 2), the S2 subunit is italicized (amino acids 683-1270 of SEQ ID NO: 2), the N-terminal domain includes amino acids 14-300 of SEQ ID NO: 2, and the transmembrane and cytoplasmic domains are italicized and underlined (amino acids 1209-1270 of SEQ ID NO: 2). The amino acid sequence of the ectodomain of the exemplary reference SARS-CoV-2 spike protein (without the native signal sequence) is set forth in SEQ ID NO: 3. The S1 subunit is boldface (amino acids 1-669 of SEQ ID NO: 3), the S2 subunit is italicized amino acids (amino acids 670-1195 of SEQ ID NO: 3, and the N-terminal domain contains amino acids 1-287 of SEQ ID NO: 3. The amino acid sequence of the RBD of the exemplary reference SARS-CoV-2 spike protein is set forth in SEQ ID NO: 1. The amino acid sequence of the immature SARS-CoV-2 Wuhan-Hu-1 Spike Reference Protein is set forth in SEQ ID NO: 4. The signal peptide (amino acids 1-13 of SEQ ID NO: 4 are underlined).

TABLE 1
Reference SARS-CoV-2 Amino Acid Sequences

| Description | Amino Acid Sequence | SEQ ID NO |
|---|---|---|
| RBD of a Reference | NITNLCPFDEVENATRFASVYAWNRKRISNCVADY | 1 |
| SARS-CoV-2 Spike | SVLYNLAPFFTFKCYGVSPTKLNDLCFTNVYADSF | |
| Protein | VIRGDEVRQIAPGQTGNIADYNYKLPDDFTGCVIA | |
| | WNSNKLDSKVSGNYNYLYRLFRKSNLKPFERDIST | |
| | EIYQAGNKPCNGVAGENCYFPLRSYSFRPTYGVGH | |
| | QPYRVVVLSFELLHAPATVCGPKKST | |
| Reference SARS-CoV- | [symbol] QCVNLTTRTQLPPAYTNSFTRG | 2 |
| 2 Spike Protein | VYYPDKVFRSSVLHSTQDLFLPFFSNVTWFHVISG | |
| | TNGTKRFDNPVLPFNDGVYFASIEKSNIIRGWIFG | |
| | TTLDSKTQSLLIVNNATNVVIKVCEFQFCNDPFLD | |
| | HKNNKSWMESEFRVYSSANNCTFEYVSQPFLMDLE | |
| | GKQGNFKNLREFVFKNIDGYFKIYSKHTPIIVEPE | |
| | RDLPQGFSALEPLVDLPIGINITRFQTLLALHRSY | |
| | LTPGDSSSGWTAGAAAYYVGYLQPRTFLLKYNENG | |
| | TITDAVDCALDPLSETKCTLKSFTVEKGIYQTSNF | |
| | RVQPTESIVRFPNITNITNLCPFDEVFNATRFASV | |
| | YAWNRKRISNCVADYSVLYNLAPFFTFKCYGVSPT | |
| | KLNDLCFTNVYADSFVIRGDEVRQIAPGQTGNIAD | |
| | YNYKLPDDFTGCVIAWNSNKLDSKVSGNYNYLYRL | |
| | FRKSNLKPFERDISTEIYQAGNKPCNGVAGENCYF | |
| | PLRSYSFRPTYGVGHQPYRVVVLSFELLHAPATVC | |
| | GPKKSTKNKCVNFNFNGLKGTGVLTESNKKFLPFQ | |
| | QFGRDIADTTDAVRDPOTLEILDITPCSFGGVSVI | |
| | TPGTNTSNOVAVLYQGVNCTEVPVAIHADQLTPTW | |
| | RVYSTGSNVFQTRAGCLIGAEYVNNSYECDIPIGA | |
| | GICASYQTQTKSHRRARSVASQSIIAYTMSLGAEN | |
| | SVAYSNNSIAIPTNFTISVTTEILPVSMTKTSVDC | |
| | TMYICGDSTECSNLLLQYGSFCTQLKRALTGIAVE | |
| | QDKNTQEVFAQVKQIYKTPPIKYFGGFNFSQILPD | |
| | PSKPSKRSFIEDLLFNKVTLADAGFIKQYGDCLGD | |
| | IAARDLICAQKFKGLTVLPPLLTDEMIAQYTSALL | |
| | AGTITSGWTFGAGAALQIPFAMQMAYRENGIGVTQ | |
| | NVLYENQKLIANQFNSAIGKIQDSLSSTASALGKL | |
| | QDVVNHNAQALNTLVKQLSSKFGAISSVLNDIFSR | |
| | LDKVEAEVOIDRLITGRLOSLOTYVTQQLIRAAEI | |
| | RASANLAATKMSECVLGQSKRVDFCGKGYHLMSFP | |
| | QSAPHGVVFLHVTYVPAQEKNFTTAPAICHDGKAH | |
| | FPREGVFVSNGTHWFVTORNFYEPQIITTDNTFVS | |
| | GNCDVVIGIVNNTVYDPLQPELDSFKEELDKYFKN | |
| | HTSPDVDLGDISGINASVVNIQKEIDRLNEVAKNL | |
| | NESLIDLQELGKYEQYIKWPWYIWLGFIAGLIAIV | |
| | MVTIMLCCMTSCCSCLKGCCSCGSCCKFDEDDSEP | |
| | VLKGVKLHYT | |
| Reference SARS- | QCVNLTTRTQLPPAYTNSFTRGVYYPDKVFRSSVL | 3 |
| CoV-2 Spike | HSTQDLFLPFFSNVTWFHVISGTNGTKRFDNPVLP | |
| Protein | FNDGVYFASIEKSNIIRGWIFGTTLDSKTQSLLIV | |
| Ectodomain | NNATNVVIKVCEFQFCNDPFLDHKNNKSWMESEFR | |
| (Lacking | VYSSANNCTFEYVSQPFLMDLEGKQGNFKNLREFV | |
| native signal | FKNIDGYFKIYSKHTPIIVEPERDLPQGFSALEPL | |
| sequence) | VDLPIGINITRFQTLLALHRSYLTPGDSSSGWTAG | |
| | AAAYYVGYLQPRTFLLKYNENGTITDAVDCALDPL | |
| | SETKCTLKSFTVEKGIYQTSNFRVQPTESIVRFPN | |
| | ITNITNLCPFDEVFNATRFASVYAWNRKRISNCVA | |
| | DYSVLYNLAPFFTFKCYGVSPTKLNDLCFTNVYAD | |
| | SFVIRGDEVRQIAPGQTGNIADYNYKLPDDFTGCV | |
| | IAWNSNKLDSKVSGNYNYLYRLFRKSNLKPFERDI | |
| | STEIYQAGNKPCNGVAGENCYFPLRSYSFRPTYGV | |
| | GHQPYRVVVLSFELLHAPATVCGPKKSTKNKCVNF | |
| | NFNGLKGTGVLTESNKKFLPFQQFGRDIADTTDAV | |
| | RDPQTLEILDITPCSFGGVSVITPGTNTSNOVAVL | |
| | YQGVNCTEVPVAIHADQLTPTWRVYSTGSNVFQTR | |
| | AGCLIGAEYVNNSYECDIPIGAGICASYQTQTKSH | |
| | RRARSVASQSIIAYTMSLGAENSVAYSNNSIAIPT | |
| | NFTISVTTEILPVSMTKTSVDCTMYICGDSTECSN | |
| | LLLQYGSFCTOLKRALTGIAVEQDKNTQEVFAQVK | |
| | QIYKTPPIKYFGGFNFSQILPDPSKPSKRSFIEDL | |
| | LFNKVTLADAGFIKQYGDCLGDIAARDLICAQKFK | |
| | GLTVLPPLLTDEMIAQYTSALLAGTITSGWTFGAG | |
| | AALQIPFAMQMAYRENGIGVTONVLYENQKLIANQ | |
| | FNSAIGKIQDSLSSTASALGKLQDVVNHNAQALNT | |
| | LVKQLSSKFGAISSVLNDIFSRLDKVEAEVOIDRL | |
| | ITGRLOSLOTYVTQQLIRAAEIRASANLAATKMSE | |
| | CVLGQSKRVDFCGKGYHLMSFPQSAPHGVVFLHVT | |
| | YVPAQEKNFTTAPAICHDGKAHFPREGVFVSNGTH | |
| | WFVTORNFYEPQIITTDNTFVSGNCDVVIGIVNNT | |
| | VYDPLQPELDSFKEELDKYFKNHTSPDVDLGDISG | |
| | INASVVNIQKEIDRLNEVAKNLNESLIDLQELGKY | |
| | EQYIK | |
| Immature SARS-CoV- | MFVFLVLLPLVSSQCVNLTTRTQLPPAYTNSFTRG | 4 |
| 2 Wuhan-Hu-1 Spike | VYYPDKVFRSSVLHSTQDLFLPFFSNVTWFHAIHV | |
| Protein | SGTNGTKRFDNPVLPENDGVYFASTEKSNIIRGWI | |
| | FGTTLDSKTQSLLIVNNATNVVIKVCEFQFCNDPF | |
| | LGVYYHKNNKSWMESEFRVYSSANNCTFEYVSQPF | |
| | LMDLEGKQGNFKNLREFVEKNIDGYFKIYSKHTPI | |
| | NLVRDLPQGFSALEPLVDLPIGINITRFQTLLALH | |
| | RSYLTPGDSSSGWTAGAAAYYVGYLQPRTELLKYN | |
| | ENGTITDAVDCALDPLSETKCTLKSFTVEKGIYQT | |
| | SNFRVQPTESIVRFPNITNLCPFGEVENATRFASV | |
| | YAWNRKRISNCVADYSVLYNSASFSTFKCYGVSPT | |
| | KLNDLCFTNVYADSFVIRGDEVRQIAPGQTGKIAD | |
| | YNYKLPDDFTGCVIAWNSNNLDSKVGGNYNYLYRL | |
| | FRKSNLKPFERDISTEIYQAGSTPCNGVEGENCYF | |
| | PLQSYGFQPTNGVGYQPYRVVVLSFELLHAPATVC | |
| | GPKKSTNLVKNKCVNFNFNGLTGTGVLTESNKKEL | |
| | PFQQFGRDIADTTDAVRDPQTLEILDITPCSFGGV | |
| | SVITPGTNTSNQVAVLYQDVNCTEVPVAIHADQLT | |
| | PTWRVYSTGSNVFQTRAGCLIGAEHVNNSYECDIP | |
| | IGAGICASYQTQTNSPRRARSVASQSIIAYTMSLG | |
| | AENSVAYSNNSIAIPTNFTISVTTEILPVSMTKTS | |
| | VDCTMYICGDSTECSNLLLQYGSFCTQLNRALTGI | |
| | AVEQDKNTQEVFAQVKQIYKTPPIKDFGGFNFSQI | |
| | LPDPSKPSKRSFIEDLLENKVTLADAGFIKQYGDC | |
| | LGDIAARDLICAQKENGLTVLPPLLTDEMIAQYTS | |
| | ALLAGTITSGWTFGAGAALQIPFAMQMAYRENGIG | |
| | VTQNVLYENQKLIANQFNSAIGKIQDSLSSTASAL | |
| | GKLQDVVNQNAQALNTLVKQLSSNFGAISSVLNDI | |
| | LSRLDKVEAEVOIDRLITGRLQSLQTYVTQQLIRA | |
| | AEIRASANLAATKMSECVLGQSKRVDFCGKGYHLM | |
| | SFPQSAPHGVVFLHVTYVPAQEKNFTTAPAICHDG | |
| | KAHFPREGVFVSNGTHWFVTORNFYEPQIITTDNT | |
| | FVSGNCDVVIGIVNNTVYDPLQPELDSFKEELDKY | |
| | FKNHTSPDVDLGDISGINASVVNIQKEIDRLNEVA | |
| | KNLNESLIDLQELGKYEQYIKWPWYIWLGFIAGLI | |
| | AIVMVTIMLCCMTSCCSCLKGCCSCGSCCKFDEDD | |
| | SEPVLKGVKLHYT | |

Provided herein are, inter alia, SARS-CoV-2 spike proteins and polypeptides (e.g., SARS-CoV-2 spike protein or polypeptide immunogens (and immunogenic fragments and/or immunogenic variants thereof)) and nucleic acid molecules encoding the same, that comprise at least one amino acid substitution described herein (e.g., at least one amino acid substitution set forth in Table 2) (e.g., at least one set of amino acid substitutions described herein, e.g., at least one set of amino acid substitutions set forth in Table 2).

While exemplary amino acid substitutions are provided herein, for example in Table 2, that specify the parental amino acid and the substituted amino acid; it is to be understood that the disclosure includes the substitution of any parental amino acid with the substituted amino acid. As such, the substitutions set forth herein (e.g., in Table 2) include the substitution of any parental amino acid with the substituted amino acid. For example, the disclosure of amino acid substitution I28Y set forth in Table 2 incudes the substitution of any parental amino acid with tyrosine at amino acid position 28 (amino acid numbering is relative to SEQ ID NO: 1).

While exemplary amino acid substitutions are provided herein, for example in Table 2, that specify the parental amino acid and the substituted amino acid; it is to be understood that the disclosure includes the substitution of any parental amino acid with the substituted amino acid or a physiochemically similar amino acid to the substituted amino acid set forth in Table 2. A person of ordinary skill in the art can determine which amino acids would be considered physiochemically similar to any given substituted amino acid set forth in Table 2 utilizing standard methods (e.g., based on the physiochemical properties (e.g., charge, polarity, etc.) of each amino acid).

The amino acid numbering utilized in Table 2 is relative to the amino acid sequence set forth in SEQ ID NO: 1. A person of ordinary skill in the art could readily determine if any SARS-CoV-2 spike protein contained one or more of the amino acid substitutions (e.g., one or more of the sets of substitutions) set forth in Table 2 through standard sequence comparisons (including, e.g., standard sequence alignments).

For the sake of clarity, while the amino acid numbering utilized in Table 2 is relative to the amino acid sequence set forth in SEQ ID NO: 1, which comprises a portion of a reference Spike protein (i.e., the RBD), it is clear that the SARS-CoV-2 spike proteins and polypeptides (e.g., immunogens (and immunogenic fragments and/or immunogenic variants thereof)) provided herein can comprise a longer amino acid sequence than the RBD (e.g., the SARS-CoV-2 spike proteins and polypeptides (e.g., immunogens (and immunogenic fragments and/or immunogenic variants thereof)) provided herein can comprise a full length SARS-CoV-2 spike protein).

TABLE 2
Amino Acid Substitutions of SARS-COV-2 Proteins
and Polypeptides (e.g., Immunogens)
SARS-COV-2 RBD Amino Acid Substitution(s)

I28Y
I28W
Y35F
Y39R
Y39A
Y39K
F44Q
F44S
F44G
F44R
F44K
F44A
F44H
F44D
F44P
F44E
F44T
F45N
F45R
F45G
F45M
F45S
F45A
F45P
F45D
F45E
F45K
F45T
F45Q
F45H
F45Y
F47W
P54W
N87L
N87H
N87M
N87Y
N87I
N87V
S116G
L125W
L125P
V153S
R163K
R163M
R163G
R163A
R163T
R163V
R163Q
L188T
L188G
H189D
A190G
P197W
K199A
N1T/F45T
N1P/F45K
N1R/F45A
N1D/L122K
N1R/R163V
I2L/A18P
I2A/I28M
I2F/A33W
I2N/A33F
I2R/Y35L
I2V/V37F
I2G/V37F
I2H/Y39N
I2V/Y39K
I2S/F44S
I2S/F45T
I2P/F45D
I2T/F45A
I2A/F45H
I2P/F45E
I2K/T46H
I2R/I72V
I2L/N87I
I2N/N120E
I2S/L125P
I2T/R163Q
I2R/R163H
I2A/P197A
I2Q/P197L
T3P/A18P
T3S/Y35F
T3D/F45S
T3Q/F45T
T3S/S116E
N4T/P54K
L5P/N24K
L5F/I28Y
L5Y/Y35W
L5P/Y35W
L5H/V37F
L5P/V37W
L5A/V37F
L5W/F44S
L5S/F44S
L5M/F44T
L5Y/F45R
L5V/F45S
L5G/F45A
L5R/Q84A
L5P/N87I
L5R/V153S
L5R/P197A
P7T/I28Y
P7R/F44D
P7S/F47V
P7A/L122K
F8W/I28Y
D9N/F45T
D9F/F45A
D9S/F45Y
D9T/F45S
D9E/P197K
D9E/K199V
E10Q/V37F
V11I/F160R
N13S/F45A
A14D/F45A
T15H/I28Y
T15P/L38I
T15S/F45Q
T15P/P197L
R16Q/F44T
R16T/F47L
R16T/N87V
R16T/V153T
R16S/A154T
R16K/R163E
R16E/A190G
R16T/P197T
A18P/N30G
A18P/S36R
A18P/Y39V
A18P/L41S
A18S/F44G
A18G/F45G
A18P/Q84S
A18P/L122G
A18P/L122M
A18P/K128R
A18P/L188R
S19P/I28F
A22N/V37S
A22D/F45D
N24K/N40K
N24V/F45S
N24S/T46S
N24R/F47M
N24T/R163A
N24E/T201P
K26R/V37S
K26T/V37K
K26L/V37F
K26V/F44G
K26T/F44K
K26T/F44S
K26I/F45D
K26V/F45P
K26Q/F47M
K26R/T100M
K26V/I104F
K26R/V153K
R27L/V37F
R27I/Y39K
R27H/F44S
R27N/F44S
R27L/F44G
R27S/F44D
R27K/N87R
R27E/N130K
R27T/F134Y
R27Q/V153S
R27I/P197V
I28M/Y35M
I28F/Y35P
I28Y/Y35N
I28F/S36Q
I28F/S36G
I28L/V37F
I28M/V37L
I28F/V37A
I28M/V37S
I28M/V37R
I28M/Y39H
I28F/Y39W
I28F/N40H
I28F/L41A
I28Y/L41G
I28F/P43S
I28W/F44S
I28L/F44R
I28A/F47I
I28F/Y50F
I28L/P54W
I28Y/P54A
I28F/N58Y
I28F/L60M
I28W/C61Y
I28Y/T63Q
I28Y/V65I
I28F/A67V
I28M/R78H
I28F/R78H
I28Y/Q84S
I28L/N87V
I28Y/Y91N
I28Y/D98E
I28L/A105W
I28F/K110R
I28M/K114T
I28M/K114N
I28L/L122M
I28Y/L122I
I28F/K128M
I28F/K132L
I28M/F134G
I28F/T140L
I28Y/P149Q
I28F/N151Y
I28F/A154L
I28W/L188K
I28Y/L188H
I28M/A192P
I28F/C195K
I28Y/C195R
I28F/C195F
I28M/S200V
I28W/T201M
S29W/F45S
S29T/P197T
N30R/F45S
V32I/Y39R
V32K/F45A
V32K/L122R
V32T/F134Y
V32R/V173L
A33P/F47M
A33W/N64A
A33W/V173D
Y35F/F44V
Y35L/F44S
Y35I/F45D
Y35L/F47T
Y35F/F62W
Y35F/N64E
Y35W/Y66T
Y35F/K114T
Y35L/L122R
Y35L/L122K
Y35L/N130K
Y35W/V153A
Y35W/E186D
Y35F/A190T
Y35W/P197A
Y35F/K198S
Y35F/K198Y
Y35W/T201L
Y35F/T201S
S36N/F45D
S36N/F47M
S36T/Q84A
S36T/R163V
V37W/A42H
V37A/F44S
V37P/F44D
V37T/F44A
V37M/F44D
V37F/F44G
V37L/F45Y
V37N/F45Q
V37Q/F45S
V37A/F45N
V37E/F45A
V37S/F45R
V37W/F47I
V37F/K48R
V37F/V52M
V37N/P54K
V37M/P54K
V37S/P54I
V37F/P54S
V37S/K56E
V37F/D59S
V37F/G83H
V37M/T85R
V37S/N87L
V37S/N87F
V37S/N87T
V37F/D97V
V37F/A105F
V37F/L111Q
V37F/K114E
V37R/L122G
V37L/L125P
V37F/K128P
V37A/N130K
V37K/N130P
V37S/F134Y
V37F/K148T
V37F/K148V
V37F/F156V
V37T/R163M
V37W/R163K
V37F/T170A
V37L/Y178H
V37F/P191R
V37Y/P197V
V37T/P197L
V37L/K198N
V37F/K198R
V37A/K198N
V37F/T201A
L38V/F44P
L38V/F44K
L38F/F45D
Y39A/F44S
Y39Q/F44Y
Y39M/F44S
Y39R/F45N
Y39Q/F45T
Y39H/F45R
Y39K/F45N
Y39S/F45Q
Y39L/F45S
Y39A/F45T
Y39D/F45D
Y39R/T46H
Y39T/T46S
Y39G/F47V
Y39L/S53Q
Y39R/N58Q
Y39A/D59E
Y39A/T85R
Y39D/N87H
Y39P/N87V
Y39K/T100L
Y39K/K110D
Y39S/L125P
Y39R/S129D
Y39L/R163H
Y39N/H175W
Y39S/L187S
Y39A/L188S
Y39D/P197R
Y39T/P197R
Y39S/P197L
Y39T/P197S
Y39H/P197I
N40R/F45G
N40L/F45A
N40R/N87L
N40D/R163H
N40S/V173L
N40R/T201L
N40R/T201V
L41T/F45R
L41K/F45A
L41S/T100F
L41V/N130K
L41T/P197L
A42D/F45S
A42G/N58P
A42G/N64E
A42R/N87F
A42Q/L125P
A42G/R163K
P43L/F45D
F44D/F47L
F44K/V52M
F44H/S53H
F44D/P54R
F44Q/P54K
F44Q/T55A
F44D/T55N
F44R/T55S
F44A/T55Q
F44G/K56E
F44G/K56N
F44G/N58S
F44D/N58E
F44H/D59T
F44A/D59S
F44D/N64H
F44D/N64F
F44A/V65I
F44A/R78H
F44G/R78A
F44E/G83F
F44G/Q84A
F44N/N87M
F44S/D90S
F44R/D97M
F44R/T100V
F44G/1104Y
F44Y/A105W
F44E/L111V
F44R/K114R
F44E/V115E
F44R/S129T
F44E/S129E
F44P/N130R
F44T/I138T
F44A/T140R
F44G/K148R
F44S/N151G
F44P/G152R
F44R/V153E
F44A/V153S
F44H/A154S
F44P/R163I
F44S/S166A
F44E/Y178F
F44S/S184W
F44T/H189T
F44Q/H189N
F44G/H189P
F44D/K198Y
F44S/K198N
F44A/T201L
F44G/T201Q
F44H/T201S
F44P/T201D
F44S/T201L
F45S/V52Y
F45G/S53M
F45A/T55K
F45A/L57G
F45S/L57M
F45D/N58P
F45R/N58P
F45R/C61R
F45S/C61Q
F45T/C61T
F45T/C61G
F45T/C61D
F45H/C61Y
F45S/F62L
F45Q/T63S
F45G/N64H
F45N/N64T
F45G/V65I
F45E/I72V
F45T/V77L
F45S/R78S
F45D/G83K
F45T/Q84G
F45Q/T85R
F45S/D90E
F45T/K94R
F45S/L95V
F45S/D97Y
F45S/D97N
F45G/D97H
F45D/D97R
F45S/D98S
F45K/T100K
F45D/T100I
F45S/T100Y
F45Y/A105S
F45G/A105S
F45S/K110S
F45A/K114W
F45A/K114Q
F45M/V115Q
F45D/S116G
F45S/Y119K
F45A/N120A
F45S/L122M
F45P/L122E
F45V/L125S
F45M/F134Y
F45G/I138V
F45D/I138T
F45D/S139A
F45A/S139H
F45T/S139K
F45S/Q144T
F45N/P149A
F45Q/G152A
F45S/G152R
F45V/V153S
F45K/V153D
F45M/V153M
F45S/V153A
F45H/V153A
F45S/A154E
F45Q/A154S
F45R/R163F
F45R/R163L
F45S/V173D
F45D/Y178W
F45R/Y178W
F45S/E186D
F45S/L187Q
F45D/H189L
F45G/H189Y
F45S/A190V
F45S/A190W
F45A/P191N
F45P/P191H
F45T/A192M
F45L/P197A
F45S/P197R
F45A/K198Q
F45E/K199Q
F45R/K199L
F45S/K199H
F45E/K199N
F45N/T201D
F45P/T201K
F45H/T201M
F45S/T201K
T46S/T55S
T46V/S116G
T46S/R163S
F47T/Y66I
F47M/Y66F
F47I/Q84A
F47L/N87Y
F47I/T100V
F47L/L122M
F47L/L125S
F47I/P149E
F47M/A154E
F47L/A154T
F47L/F160Y
F47V/T201L
G51N/R163G
V52I/R163A
V52F/R163M
P54R/V65I
P54W/R163K
P54R/K199R
P54K/K199N
T55A/L188T
T55W/L188G
K56E/T100Y
K56R/P197M
C61A/R163A
T63S/L188K
T63S/H189D
V65I/R163A
V77L/N87I
V77L/S116G
V77L/H189D
R78P/R163A
Q84A/D90Q
Q84A/V153H
Q84A/V153R
Q84A/R163G
Q84A/K199I
N87I/L122A
N87I/L122V
N87V/K128P
N87I/K128T
N87L/S129N
N87V/N130R
N87L/N130Q
N87M/N130R
N87H/A154T
N87M/R163K
N87T/R163V
N87L/R163V
N87Y/V173R
N87I/V173A
N87T/Y178H
N87F/K199Y
N87F/T201L
N87V/T201A
L95V/P197R
T100L/L122K
T100H/R163A
T100Y/L188G
I104M/R163V
A105S/R163A
A105S/P197A
K114R/N130K
V115P/K198R
S116G/K128S
S116N/V153S
S116D/P197C
S116G/K198G
S116G/T201K
L122Q/N130A
L122K/V173A
L122K/L187V
L122K/A192T
L122K/T201P
Y123S/L188S
L125P/K199S
K128S/L188T
S129E/R163L
S129D/R163Q
N130K/P197A
N130K/T201Q
F134Y/R163L
F134Y/P197W
K148Q/R163V
K148E/R163S
K148E/L188T
P149A/V153S
V153I/R163A
V153S/K198G
A154P/R163T
F156P/R163I
R163M/S164Y
R163Y/V173M
R163V/V173P
R163L/L188S
R163G/H189S
R163Y/A192P
R163I/P197R
R163I/P197S
R163Q/K198T
R163G/S200T
R163H/T201Q
R163A/T201W
S164N/L188S
V173I/H189D
P197A/K198R
P197S/S200K
P197S/T201M
N1P/I28M/L188N
N1G/F44Q/L122K
N1R/F44R/F160H
N1T/F44G/V173K
I2V/N13L/F45H
I2R/A18P/K26T
I2C/K26I/F45G
I2V/I28L/F45T
I2A/A33S/F45A
I2S/Y35W/F45E
I2N/Y35F/L60M
I2A/V37T/F45E
I2P/V37T/R163M
I2S/Y39T/F47C
I2L/F44G/D59S
I2A/F44S/I72V
I2R/F44S/T201G
I2R/F45R/S116T
I2S/F45G/V153M
I2R/F45H/P191T
I2L/D59E/T85R
I2F/L125W/G152N
T3I/I28F/H189N
T3M/F44E/R163Y
T3S/F44D/T201S
T3G/F45S/F134N
T3P/F45S/A192W
T3S/F47I/H189N
N4Q/N87F/N130K
L5G/R16K/F45T
L5Y/K26R/R163T
L5S/F44G/P191T
L5F/T46R/N87M
L5P/F47L/R163T
L5T/T55G/L122K
L5P/N87T/T201W
L5P/L125G/P197S
C6I/N13M/P169C
P7S/R16K/F44A
P7S/I28F/K148R
P7N/Y39N/T46S
P7D/F45H/L111Q
P7R/S53K/R163A
F8L/V65L/K114T
D9R/A18S/F45A
D9L/R27L/F45E
D9G/I28F/V52T
D9A/V37G/F45S
D9E/F44P/K148D
D9W/F45S/I104M
D9G/F45A/Y123H
D9S/F45S/V173R
D9N/V153E/H189G
E10A/F45P/V77L
E10A/N64A/N130K
V11L/F45D/L187G
T15A/I28M/D97E
T15S/F45S/D90R
R16K/S29T/N130A
R16K/V37F/V52L
R16K/V37A/T55D
R16W/F44R/R163A
R16V/L125M/F160R
A18H/N24T/P197I
A18P/I28L/K56Y
A18S/F45E/P197C
A18P/D97K/S129G
A18P/K132A/V173R
A18P/A190R/S200Y
N24R/Y39W/R163V
N24S/F44K/L60Y
N24S/F44D/S164R
N24D/F45S/T201K
K26R/N87Y/T201Q
R27L/A33W/C61S
R27Q/V37F/K128A
R27P/Y39L/F45G
R27Q/F44H/D97L
R27K/F45S/N58A
I28F/Y35H/K198T
I28Y/S36R/V52I
I28F/S36Q/Y178V
I28F/V37L/K56D
I28Y/V37K/D59A
I28M/V37S/A190V
I28L/Y39R/F44K
I28F/Y39R/F45L
I28F/Y39V/V173S
I28M/N40R/F47A
I28W/F44S/G174A
I28M/F44Y/E186S
I28L/F45M/S53L
I28L/F45A/T55N
I28F/K48E/R163L
I28F/P54R/T140C
I28F/D59M/R136K
I28Y/C61Y/S184T
I28L/Y66F/N87V
I28M/D97L/N151K
I28F/D97G/T201G
I28M/S129R/A154P
I28Y/N130I/P191T
I28Y/L131V/G152N
I28F/S184T/S200M
S29T/N40H/V115P
S29R/F45Y/P197Q
N30S/A33V/F45G
N30S/S36N/F45E
V32N/Y39L/F45A
V32L/F44A/V173E
V32I/F45V/R163K
V32K/N87I/A154S
V32T/A154P/L188A
A33W/V37K/K128R
A33W/F47M/V173L
A33L/T63K/V153S
A33W/Q84A/T100I
Y35W/F44G/N58K
Y35F/F44A/Q84E
Y35L/F44S/T140L
Y35S/F44N/S184G
Y35P/F45G/N64T
Y35W/T85A/K198A
Y35W/N151M/K198G
S36C/T55L/T201E
S36T/N87F/Y178H
S36R/I138T/T201R
V37W/L38I/I72V
V37K/F44G/T63S
V37L/F44G/F160P
V37A/F45P/T100I
V37F/F45R/H175F
V37T/F45A/A192R
V37K/F45A/T201L
V37L/F47L/R163Q
V37S/F47V/R163S
V37F/G51H/F62W
V37F/T55V/N130V
V37W/L60Y/K148S
V37R/N64E/N87Y
V37Q/V77L/P197L
V37K/R78D/R163M
V37K/N87Y/R163A
V37F/T100L/K198R
V37Y/F134Y/R163A
V37F/G152R/K198A
V37Y/R163T/T201L
V37R/Y178F/L187R
Y39I/F45E/L60A
Y39R/F45S/C61K
Y39A/F45Q/T63S
Y39L/F45A/L122V
Y39E/F45T/K128D
Y39L/F45P/S129D
Y39E/F47I/R163S
Y39V/N87I/S129K
Y39K/N130K/R136Q
N40T/F47S/P54I
N40S/F134Y/P149N
N40H/A190G/T201L
L41T/S116R/P197L
A42G/P54Q/K199P
A42M/N87Y/N120D
A42P/L111V/P197V
F44P/Q84A/V173S
F44S/Q84G/T201S
F44H/N87S/I138L
F44P/L95I/T201R
F44G/V115P/P197T
F44T/Q144N/R163A
F44W/R163G/L188G
F44S/R163T/S200Q
F44D/V173T/T201V
F45T/Y50W/K128H
F45K/T55S/R78G
F45T/N58R/K198W
F45A/C61S/V115M
F45D/F62V/T201L
F45E/T63Y/T201V
F45G/N64S/T100V
F45A/N64P/T140S
F45N/V77L/D98N
F45M/R78T/R163G
F45A/I80V/A192I
F45H/F99W/A192T
F45S/T100M/I104V
F45H/K114L/L125G
F45V/K128S/V153K
F45T/N130L/A154S
F45A/N130C/T201W
F45C/F134Y/R163V
F45P/S139D/L188C
F45T/N151M/A190S
F45T/F160A/T201W
F45M/R163Y/P191K
F45K/V173M/T201Q
F47C/P54L/Y123K
F47I/K56E/V173L
F47M/Q84M/S200R
F47I/T85R/A192F
F47Q/I104F/A192V
F47M/L187M/T201L
K48T/R163V/S200L
P54I/C61R/L122G
T55A/I104F/L188T
T55D/Y123F/K198A
N58G/L122K/P197K
T63K/L125W/A192T
N64V/Y123K/L188S
T85M/R163V/P197V
N87H/D97A/V173D
N87K/T100K/L122E
N87C/R163V/P197R
N87F/L187R/K199H
L122K/S129A/T201Q
F160M/P197V/T201L
R163V/H175S/P197V
N1T/K26R/F45S/E141T
N1C/I28F/C61E/I104A
N1L/V37A/F45T/L188K
I2Y/F45V/N87Y/R163S
I2N/F45A/D98T/A192P
I2V/Q84A/S166G/K199I
I2H/N87R/F156P/R163M
N4L/A18P/F45S/S69R
N4R/L125A/V173L/H175Y
L5P/F45G/K56S/K199G
C6S/A14S/F45P/V52T
D9H/A18S/F45T/T100V
A14S/L38F/F44E/K148N
T15E/A22N/I28L/P54R
T15E/V37N/F45E/T201Y
T15R/Y39R/L57I/N87I
T15P/F45A/G83F/L187W
R16Q/R27K/T46E/S129G
A18P/F45M/D59S/Q84R
A18Q/F45T/I104L/K148Q
A22V/V37F/F44E/S129R
A22S/Y50I/D112R/Y159R
R27V/F44S/I138N/A192G
I28F/A42R/N58R/D90E
I28M/F44A/C61W/K199C
I28F/F45R/Y91L/T201W
I28Y/V115T/Y178H/A190T
V32S/F45G/C61F/K198Y
V32T/F45S/P149L/S200F
D34E/V37I/F45A/N120R
V37M/A42G/I72V/R78T
V37R/F45A/D97I/H189P
V37S/K48R/L188S/A190E
V37F/K56S/Y119P/S139R
V37F/A145R/V153M/R163T
Y39L/F45S/C61A/N87H
Y39N/F47V/L111E/R163L
Y39A/P54K/N130P/V153D
Y39A/A105T/S139T/P197W
L41P/F47L/T55S/N87M
F44G/P54W/D59V/V173R
F44H/N58P/A154T/V194A
F44E/C61Q/R163S/L187P
F44H/T85S/K128P/A190T
F44S/R163I/L188N/T201A
F45Y/N64T/L111A/A190G
F45S/N87C/I142V/V153E
F47W/P54W/T85M/P197L
C61N/R78S/S116V/R163V
I2Y/D9S/I28Y/N40R/N58K
N4S/N24T/N87H/L111Q/A192T
N4D/F45S/Y50F/K56N/N64L
L5P/V37Y/L111E/S129K/T201G
L5P/F44D/N151H/V173K/L188H
P7I/A22S/Y39Q/F45E/T55K
D9G/L41S/P43S/F45S/N87K
E10A/A33V/V37T/F45S/P149C
F12S/F17S/Q79V/C158S/R179
T15P/F45S/Y91L/L125G/V153G
A22D/V37F/L95I/K148T/A190G
N24T/S36K/F45P/C61F/A154P
K26R/F45H/S116E/G155R/V173E
Y39N/F45T/T85M/N130P/A190G
Y39P/F45S/S116L/Y123S/T201R
Y39S/T100L/I104F/L122A/F160R
A22Q/N30S/V37S/F45S/V173L/T201V
Y35F/A42T/Y123K/Y178H/S184H/L188P
Y39M/F45E/P54R/Y66T/D97R/F134Y/F160M/L187P
L41S/P43S/F45S/N87K/K110N/S116G/N147S/K148T/A154E/
R163Q/S166G/R168Q/Y171N/H175Y
D9G/L41S/P43S/F45S/N87K/K110N/S116G/N147S/K148T/
A154E/R163Q/S166G/R168Q/Y171N/H175Y
K26R/L41S/P43S/F45S/N87K/K110N/S116G/N147S/K148T/
A154E/R163Q/S166G/R168Q/Y171N/H175Y
D9G/L41S/P43S/F45S/N87K/K110N/S116G/N147S/K148T/
A54E/R163Q/S166G/R168Q/Y171N/H175Y/K199L
L5K
P7N
R16T
R16Q
R16E
R16K
F17W
N24E
N24Q
I28L
A33F
L38I
Y39N
Y39E
Y39H
Y39S
N40R
L41Y
L41H
F44N
F45V
F45C
T46H
T46E
T46Q
F47I
F47V
F47M
P54M
T55E
F62W
V65I
V65M
N87T
N87F
N87K
A105W
S116D
S116E
L122R
L125S
L125G
K148E
V153T
A154E
F160E
F160H
R163I
R163Y
R163F
R163L
R163S
R163H
S166G
S166A
V173E
V173Q
L187Q
L187N
L187S
L188D
L188K
P197H
K199F
N1T/D9
N1S/I28M
N1D/F44S
N1S/L188T
N1P/H189D
N1S/P197L
I2A/I28L
I2S/I28W
I2M/A33F
I2N/A33W
I2T/V37S
I2L/V37W
I2S/V37A
I2A/V37S
I2L/Y39F
I2S/Y39M
I2P/Y39A
I2T/Y39S
I2D/F44S
I2S/F44R
I2Y/F45S
I2Y/F45K
I2L/T46H
I2S/T46M
I2V/F47V
I2V/P54R
I2C/P54A
I2L/D59E
I2V/R78S
I2P/N87V
I2R/L122R
I2G/L125S
I2L/N130R
I2C/R163E
I2C/R163N
I2C/S166G
I2S/P197H
T3S/V11I
T3N/I28W
T3S/Y35L
T3A/Y39R
T3A/F44D
T3P/F45R
T3S/N87H
T3A/P197L
N4T/R16N
N4S/R16H
N4D/I28F
N4R/A33F
N4T/V37F
N4D/F45C
N4R/N87P
N4A/N87I
N4V/R163V
N4R/T201S
L5N/R16T
L5T/N24R
L5T/I28M
L5P/V37R
L5M/L38I
L5H/Y39L
L5K/Y39G
L5F/F44G
L5N/N87T
L5V/I104F
L5E/L125G
L5Q/L188G
L5V/P197A
C6M/W106
P7H/Y39A
P7A/N87H
F8I/I28F
F8L/F44P
D9H/F44R
D9Q/F44S
D9P/F45H
D9E/N87P
D9A/T201L
E10D/F44Q
E10S/F44S
V11I/Y39T
V11I/R136G
F12L/A18P
N13H/I28M
N13S/I28M
N13R/F45G
N13H/F45S
N13H/T46H
N13Y/P197L
T15P/Y35L
T15R/Y39R
T15V/F45N
T15S/S129
R16N/I28L
R16K/S29H
R16L/F44S
R16A/F45K
R16E/T46R
R16S/F47I
R16K/I72V
R16H/R78D
R16S/N87P
R16S/V173L
R16T/L188S
R16E/K198R
F17W/K110D
F17D/P161T
F17W/S200W
A18P/L38M
A18S/F44S
A18G/F45E
A18P/Q84K
A18P/Y121L
A18S/V153S
A18D/S166F
A18P/L188P
A18P/C195N
V20A/F45S
N24R/Y39L
N24Q/L41R
N24H/A42G
N24K/A42V
N24S/N58P
N24K/L125A
N24Q/K128P
N24R/S129G
N24Q/L188R
N24E/L188R
N24T/L188S
N24T/S200V
R25L/G74
K26T/V37Q
K26V/V37A
K26T/Y39R
K26R/A42G
K26R/F47I
K26R/F47V
K26R/V52I
K26R/T55H
K26V/L125G
K26T/V153Q
K26R/L187S
K26V/P197S
R27W/V37F
R27Q/L38I
R27Q/N40K
R27A/F45R
R27Q/D59E
R27Y/Y66T
R27K/D98N
R27V/K148T
R27K/A154S
R27L/R163M
R27V/H189G
R27V/A192G
R27V/P197A
R27V/T201W
I28M/Y39C
I28Y/A42E
I28F/C61Y
I28L/C61N
I28M/Y66F
I28M/R78S
I28L/N87T
I28M/D90E
I28M/D90S
I28M/D97R
I28F/D98Y
I28Y/V153W
I28M/S164N
I28L/L188H
I28W/A192L
I28L/K198S
S29H/Y39S
S29P/A42G
S29P/F45Q
S29T/F160H
N30G/F44P
N30K/Y165E
V32A/F45E
V32R/S53D
V32I/N87H
V32T/Y121L
V32T/K198R
V32T/K199W
V32I/T201V
V32I/T201W
A33V/F47I
D34T/F45S
D34S/F45T
Y35W/P43D
Y35S/F44H
Y35F/K56Q
Y35W/L60R
Y35L/F62W
Y35W/N64W
Y35L/V65I
Y35W/D98V
Y35I/L122S
Y35F/L122V
Y35I/N130K
Y35F/E141T
Y35W/Q144M
Y35L/V173T
Y35L/V173R
Y35L/V173D
Y35L/L188Q
Y35L/A192Q
Y35L/T201P
S36G/F45C
S36T/T201R
V37R/A42Q
V37A/F44L
V37Y/F44T
V37R/F44R
V37R/F45L
V37H/F45M
V37Q/F47V
V37F/S53G
V37A/T55Q
V37M/K56Q
V37M/K56A
V37S/N64P
V37S/N87P
V37A/N87R
V37Y/N87M
V37L/N87T
V37F/N109K
V37M/S116D
V37T/Y119H
V37S/Y119N
V37A/L122Y
V37F/K128W
V37L/N130R
V37W/N130V
V37M/F134Y
V37M/A154T
V37A/F160S
V37T/R163I
V37R/V173T
V37M/V173R
V37F/V173G
V37T/H189S
V37I/A190S
V37W/A192L
V37M/A192N
V37F/K198A
V37H/K198G
V37R/K199S
V37K/S200M
V37M/T201M
V37A/T201R
V37W/T201G
V37R/T201L
L38R/C61I
L38I/N87A
L38Q/N107
L38I/N120G
Y39I/F45T
Y39W/F47Y
Y39M/F47I
Y39S/F47M
Y39K/V52I
Y39Q/T55S
Y39D/T55R
Y39R/T55N
Y39N/N58K
Y39M/V65L
Y39D/N87L
Y39Q/Y91F
Y39R/Y91F
Y39Q/D97S
Y39W/D98N
Y39L/D98N
Y39A/S116K
Y39Q/Y119N
Y39R/Y119N
Y39V/S129K
Y39S/T140S
Y39S/N151E
Y39L/N151G
Y39Q/V153M
Y39R/V153S
Y39W/A154S
Y39L/F160K
Y39T/S184T
Y39K/L187H
Y39V/L188S
Y39K/A190R
Y39S/A192G
Y39V/P197V
Y39W/K198R
Y39M/K198R
Y39G/S200T
Y39H/T201H
Y39M/T201L
N40R/F45K
N40S/F45A
N40K/T46H
N40K/F47L
N40K/N120K
N40R/L125S
N40R/V153T
N40P/R163G
N40S/H175Y
N40R/H189S
N40H/P197K
N40R/K199W
N40S/T201L
L41V/K48R
L41S/T55Q
L41S/R78P
L41S/N87H
L41A/I104Y
L41R/F160N
L41S/F160K
L41T/R163V
L41A/L188S
L41Y/A192G
L41H/K199M
L41M/T201M
A42S/F45R
A42P/F47V
A42G/D59Q
A42V/N64V
A42G/V115L
A42G/S116R
A42G/L122R
A42G/T140M
A42G/P149M
A42G/P197E
A42G/K198S
A42G/K198T
A42K/T201G
P43R/Q84A
P43Q/N87H
P43R/L122R
P43N/R163V
F44N/P54W
F44N/P54R
F44Y/T55H
F44R/T55N
F44T/K56E
F44E/K56R
F44P/N58R
F44S/D59R
F44M/D59E
F44H/V71I
F44S/T85V
F44L/N87F
F44R/D90N
F44S/T100I
F44E/T100A
F44V/T100F
F44E/I104L
F44N/A105S
F44H/K110M
F44H/S129N
F44S/L131V
F44K/S139G
F44T/P149H
F44A/V153L
F44S/A154D
F44M/A154T
F44S/F160G
F44P/V173E
F44D/V180I
F44G/H189V
F44T/H189S
F44H/H189Y
F44S/T201
F44G/T201W
F44T/T201M
F45H/P54D
F45S/L60S
F45D/F62C
F45H/F62N
F45S/N64R
F45K/I72V
F45Y/V77L
F45P/G83M
F45S/N87T
F45Y/D97T
F45S/D98P
F45E/T100Y
F45S/T100S
F45A/T100K
F45K/A105T
F45Q/V115L
F45P/Y119H
F45P/Y119E
F45A/Y119A
F45D/L122H
F45T/K128V
F45D/K128M
F45S/S129Y
F45T/N130M
F45D/N130I
F45M/N151A
F45Y/N151S
F45P/V153D
F45S/A154L
F45R/A154V
F45A/G155R
F45A/Y178T
F45V/Y178W
F45G/S184K
F45C/A190T
F45D/P191L
F45P/A192T
F45M/A192K
F45G/K198G
F45Y/K199I
F45N/T201M
F45Y/T201L
T46R/S116N
T46H/N120E
T46S/R163I
T46Y/V173I
T46S/L187R
T46H/H189P
F47I/T55R
F47M/D59K
F47W/L60Y
F47L/N64T
F47T/N87T
F47V/A105T
F47V/V115R
F47V/Y119N
F47I/K148T
F47I/V173R
F47W/A190S
F47I/K198G
F47W/T201M
F47V/T201S
K48N/L125P
K48R/T201S
Y50F/Y178N
V52L/N87Y
V52F/S116G
S53D/S139T
P54A/R78S
P54A/T85S
P54D/S116G
P54Q/F134Y
P54E/F160Q
P54W/L188V
P54R/A192G
P54M/P197S
P54R/K198P
T55A/T100F
T55H/I104W
T55H/S129E
T55L/R163F
T55S/R163N
T55S/R163T
T55S/P197S
T55F/P197K
T55H/K199G
T55A/T201R
T55S/T201K
K56S/R163H
K56L/R163G
K56V/P197V
N58Q/F160Y
N58S/R163T
N58P/L188G
N58P/H189N
D59S/L122R
D59S/L188S
D59E/H189Q
D59E/T201M
L60H/D98G
L60H/N130K
L60T/R163A
L60S/L187Q
C61Q/R163L
C61L/R163A
F62W/T100I
N64S/V65L
N64T/V153S
N64H/F160R
N64S/P197L
N64T/T201R
V65I/Q84M
V65L/N87F
V65I/I104W
G74V/A105P
D75S/L122Q
R78S/A105Y
R78A/R163T
A81T/A89R
G83Y/R163A
Q84A/K128S
Q84A/Y178F
Q84A/A192Q
T85Q/L95
T85S/R163K
T85R/T201S
N87H/I104M
N87T/K114T
N87H/L122G
N87L/S129A
N87F/S129T
N87T/Y178F
N87I/Y178I
N87T/H189P
N87T/P197R
N87P/P197R
N87Y/K198R
N87K/T201G
N87V/T201G
N87I/T201R
D97A/L122R
D98N/N130P
D98N/P197A
T100V/L122S
T100L/L125W
T100L/N130R
T100I/N130P
T100I/R163K
T100K/A190G
T100F/S200L
I104Y/I138T
A105Y/S116E
A105T/L122Q
A105S/T201M
L111D/L188S
L111Q/S200A
K114Q/R163L
K114T/H175Y
K114R/V182G
K114R/A190G
S116G/R163C
S116G/G196S
S116D/T201Y
Y119D/L188Q
Y119N/K198E
N120D/H175Y
N120D/L187G
Y121I/K128
L122M/K128H
L122K/I138K
L122R/I138L
L122S/K148E
L122R/V173R
L122S/E186D
L122S/P191T
L122T/S200W
L122K/T201C
L122S/T201R
Y123F/F134G
Y123F/V153H
Y123K/V173L
Y123F/T201V
L125G/A154L
L125A/Y178F
K128N/A154E
K128V/R163V
K128A/V173E
K128H/A192R
S129R/V173L
N130K/G152R
N130S/L188D
N130T/L188G
N130P/A192Q
N130S/P197T
N130R/T201E
F134S/T201A
F134Y/T201A
P149L/R163V
N151K/H175Y
N151L/L187G
G152D/A190G
V153T/K198R
F160Y/V173E
F160Y/L187M
F160A/P191G
R163S/V173T
R163H/S184G
R163L/H189T
R163A/H189G
R163K/P191R
R163T/A192V
R163K/K198S
R163Q/K199I
R163L/K199M
R163Y/K199T
R163A/T201Y
R163H/T201P
R163H/T201R
S166A/L188S
V173I/S200L
L187R/P197T
L188K/A192H
H189G/T201P
H189N/T201V
A190G/T201S
A192L/P197L
A192L/P197S
A192H/K199I
A192P/K199Y
A192Y/T201M
A192P/T201W
P197L/T201S
N1K/P7N/A42P
N1R/I28Y/Y123S
N1S/Y39T/S166G
N1V/F45G/A190V
I2T/Y21H/F44G
I2S/V32T/N40R
I2V/Y35T/F45H
I2E/Y39M/K199I
I2Y/N40S/T201N
I2H/F44E/S164T
I2D/F44H/K198D
I2N/F45N/K114M
I2K/F45L/V115K
I2V/F47T/V65I
I2H/F47T/S184H
I2F/T55A/T100L
I2P/N87L/L188R
I2A/N87H/K198M
T3E/V37F/D75Q
T3K/D59S/N130K
N4A/V37S/N87R
N4K/Y39M/K114R
N4S/F44A/P54A
N4A/F44K/D97M
N4R/F47I/K198L
N4T/S53E/T201W
N4G/N130R/R163S
N4V/A145G/P197L
L5S/N24T/V37W
L5R/S29R/R163S
L5P/V32T/N87K
L5S/V32R/T201E
L5R/Y35F/C61V
L5I/V37F/T170G
L5V/V37M/V173I
L5R/F47L/T201M
L5H/Q144A/R163A
L5A/R163S/T201H
L5R/V173K/S200A
P7E/Y35L/F44Y
P7D/T46M/N130K
P7N/L125S/V153F
P7S/R163T/A192C
F8L/I28L/V37W
F8L/Y39V/F44A
D9E/I28W/T46I
D9S/V37L/A154E
D9Y/Y39K/S200M
D9S/F45N/L122S
D9V/L95T/V103
E10K/I28F/S53I
N13Q/I28F/Y35P
N13M/V37F/T201S
T15E/T46I/R163M
R16T/F47T/P54A
R16D/T55D/1104F
R16E/Y119D/N130R
F17Y/L41M/E141G
A18P/D59Q/R78P
A18S/R78P/N87T
N24D/V37T/F44G
N24R/P54M/S200G
N24T/N87Y/D97E
K26R/Y35L/K198V
K26V/F44Y/T201R
K26R/F45L/P197T
K26R/Q84A/L125S
K26V/S129G/Y178H
R27Y/D34R/D59
R27S/Y35W/T201D
R27K/Y39M/D59Q
R27M/Y39L/P197A
R27K/L41S/S129R
R27K/F44A/T55P
R27K/T46L/T100Y
I28M/N40E/R78H
I28F/P43Q/T63D
I28F/F44V/S116P
I28M/F47T/T63H
I28W/V52F/V65I
I28M/L60V/N151E
I28F/F62L/L131V
I28F/I88V/D97G
I28F/E141L/R163L
S29R/P54W/K198E
Y35F/A42H/K128Q
Y35A/F44S/T170S
Y35M/P54A/K148E
Y35W/T55N/K56L
Y35L/N58K/K132M
Y35F/V173D/L187R
S36T/K128N/R163M
V37F/F44Y/V52A
V37L/T46E/F160E
V37E/N87T/L122G
V37S/V115K/A192Y
V37I/I138T/R163S
V37T/T170A/Y178F
V37W/L187S/T201K
V37R/A192T/P197A
L38F/F44R/K56S
L38I/N120D/K199L
Y39F/F44R/D98P
Y39R/F45D/G155R
Y39T/T46K/K148L
Y39S/T63Q/N87Y
Y39L/V65L/V173R
Y39T/R78S/F160T
Y39V/Q84A/D97K
Y39T/N92L/G152R
Y39S/T100V/S139T
Y39D/N130T/F160A
Y39L/P169C/F185Y
N40R/L95F/P177I
L41V/F45A/G83L
L41M/K48Q/P197C
L41M/T63S/L122R
L41H/T100L/T201S
L41H/F134Y/A154L
A42D/F44A/T85R
A42G/Q84M/A105S
A42V/L125P/T201V
F44S/S53W/R163M
F44S/T55M/T85A
F44T/N64P/K198D
F44A/F99W/K128R
F44G/Y119N/S129G
F44D/L122S/Q144R
F44E/I142V/T201R
F44R/S184C/K198G
F44K/T193Y/V194G
F45C/P54I/T85S
F45M/P54A/V153D
F45Y/K56R/A105S
F45Q/D59Y/T201A
F45L/L60M/T100F
F45E/T63N/R78L
F45D/N64Y/Y119A
F45S/Q84V/E141M
F45A/Q84E/V173L
F45K/A105S/K132M
F45S/Y119G/F160P
F45S/T170K/L188S
T46S/Q84M/K198M
T46L/T100F/P197R
F47T/L60M/S116N
F47I/T100L/P191N
K48S/A190P/T201V
S53I/C61S/A8I
T55S/N87I/E186N
K56R/R78S/K199A
N58Q/D98N/L188G
D59Q/F160D/K198L
D59G/P197I/T201R
C61V/R78V/G83
F62W/G83F/A190S
F62S/F156P/R163T
V65A/A105W/R163T
E76G/G101A/Q144S
V77L/Y123F/N130V
A81T/G101I/G152F
Q84A/N120F/L188T
Q84S/V153S/S200V
D98N/L187G/T201A
N120D/A190S/K199S
L122K/K128S/P149Q
K132V/V153T/P197W
V153M/F160V/S166G
A154E/S164P/T201L
L188S/A192L/S200H
N1V/R27I/F44E/V77L
I2G/E10T/Y35W/K56N
I2D/I28F/G83N/S116E
I2P/V37R/F44E/K148E
T3P/F45T/V52M/T201R
T3I/F62N/R163M/H189K
N4K/I28W/N58K/K128G
N4P/N40G/F45H/K198S
L5H/F47C/N87T/L122K
L5P/T55D/V65A/N87L
C6N/F17P/Y93V/G174
P7H/F17W/L60Q/L122R
R16K/V32C/I104F/K199A
R16S/V37F/C61T/S129A
R16G/A42R/F62W/L188S
R16D/F44A/G83Y/I104F
R16Y/F45L/N87Y/I138L
R16N/V77L/R163V/A192T
A18P/Y35P/D59A/V77L
A18R/F45A/N64E/K198V
A18P/F47S/G83N/T140A
A18P/N58P/N64K/T100Y
S19L/D34S/G83S/L95V
S19P/V37F/F44V/R78P
N24D/A42Q/R163G/T201C
K26R/Y35W/A67V/Y119Q
K26R/V37I/P54A/K199T
K26R/Y50W/K148L/L188K
I28L/L122G/A190V/K199M
S29N/F45S/A67L/Y119R
A33V/F44D/F62V/T201C
A33F/F44T/A67L/T201N
Y35W/F47R/Y119H/A145S
Y35L/Q84P/F99S/R163V
S36L/T55L/L188T/P197L
Y39R/K48Q/L122S/P197S
Y39Q/E76Y/N107G/E135
N40K/K132R/V153T/S200Y
L41R/F47V/T100L/L187V
A42S/T55S/N87M/S200G
A42G/T55H/I104M/F160H
A42N/D97R/Y178N/L183W
F45N/V77L/S129G/T201E
F45D/K128H/Q144T/K198N
T46L/F70V/I80S/F99Y
F47S/L125P/V153A/P191G
P54I/N87I/P149R/K199S
T55S/F62V/V153M/R163M
T100A/Y123F/N130K/K199V
T100N/S129N/H189D/K199Y
K128R/P149E/R163G/H189N
N1R/F45G/E186N/T193Y/V194G
I2T/Y39S/T46S/V173T/L188P
N4D/Y35F/F44S/S116Y/K199R
F12L/R27V/Y35V/F45A/N58A
T15S/L41P/F47S/R163H/T201L
R27E/L38T/C49G/N64H/I80
L41G/T55N/S129T/L187S/T201I
L41K/L125S/V153K/A192K/T201Q
V20L/L57R/G74T/G83V/W106G/R168
L41M/P54L/S69Y/K94C/G117S/C150
F44D/N87W/N120H/T140S/P149E/P197R
N4T/N30T/V37A/V52N/K94Q/D98V/H189Y
R16N
A18P
N24K
N24R
K26R
R27K
I28F
I28M
V32T
A33W
Y35W
V37S
V37W
V37F
Y39L
Y39M
Y39F
Y39Q
Y39W
Y39T
A42G
S53D
S53E
P54R
P54K
Q84A
T85H
I104F
V115P
L122Q
L122K
L122M
L122S
Y123F
N130R
N130P
N130K
F134Y
F160N
L188S
L188N
L188A
P197S
P197N
P197F
P197I
P197L
P197Y
P197A
P197R
P197M
P197V
P197T
K198N
K199V
Y39L/T201M
N40R/P197A
V11I
R16S
R16H
R27V
S29T
Y35L
V37M
V37T
V37Q
V37K
V37L
V37I
V37A
N40K
N40Q
A42R
A42K
T46S
T55S
T55H
N58P
D59E
N64A
N64E
R78A
T85R
L122T
L122A
Y123K
S129E
N130A
V153K
V153R
V153E
V153Q
F160R
V173K
V173L
S184T
L187T
H189S
H189N
P197Q
P197K
K198G
K199Y
K199S
K199G
V37T/T201S

In some embodiments, the SARS-CoV-2 spike protein or polypeptide is an immunogen (a SARS-CoV-2 spike protein or polypeptide immunogen). In some embodiments, the SARS-CoV-2 spike protein or polypeptide comprises an immunogen (a SARS-CoV-2 spike protein or polypeptide immunogen). In some embodiments, the SARS-CoV-2 spike protein or polypeptide comprises or consists of an immunogenic fragment of a SARS-CoV-2 spike protein. In some embodiments, the SARS-CoV-2 spike protein or polypeptide comprises or consists of an immunogenic variant of a SARS-CoV-2 spike protein. In some embodiments, the SARS-CoV-2 spike protein or polypeptide immunogen comprises or consists of an immunogenic fragment of a SARS-CoV-2 spike protein. In some embodiments, the SARS-CoV-2 spike protein or polypeptide immunogen comprises or consists of an immunogenic variant of a SARS-CoV-2 spike protein. In some embodiments, the SARS-CoV-2 spike protein or polypeptide immunogen comprises or consists of an immunogenic fragment of a SARS-CoV-2 spike protein immunogen. In some embodiments, the SARS-CoV-2 spike protein or polypeptide immunogen comprises or consists of an immunogenic variant of a SARS-CoV-2 spike protein immunogen.

In some embodiments, the amino acid sequence of the SARS-CoV-2 spike protein or polypeptide (e.g., a SARS-CoV-2 spike protein or polypeptide immunogen (or immunogenic fragment and/or immunogenic variant thereof)) comprises an amino acid substitution at any one or more of the amino acid positions set forth in Table 2 (e.g., an amino acid substitution at any one or more of amino acid positions N1, I2, T3, N4, L5, C6, P7, F8, D9, E10, V11, F12, N13, A14, T15, R16, F17, A18, S19, V20, Y21, A22, W23, N24, R25, K26, R27, I28, S29, N30, C31, V32, A33, D24, Y25, S36, V37, L38, Y39, N40, L41, A42, P43, F44, F45, T46, F47, K48, C49, Y50, G51, V52, S53, P54, T55, K56, L57, N58, D59, L60, C61, F62, T63, N64, V65, Y66, A67, D68, S69, F70, V71, 172, R73, G74, D75, E76, V77, R78, Q79, I80, A81, P82, G83, Q84, T85, G86, N87, I88, A89, D90, Y91, N92, Y93, K94, L95, P96, D97, D98, F99, T100, G101, C102, V103, I104, A105, W106, N107, S108, N109, K110, L111, D112, S113, K114, V115, S116, G117, N118, Y119, N120, Y121, L122, Y123, R124, L125, F126, R127, K128, S129, N130, L131, K132, P133, F124, E135, R136, D137, I138, S139, T140, E141, I142, Y143, Q144, A145, G146, N147, K148, P149, C150, N151, G152, V153, A154, G155, F156, N157, C158, Y159, F160, P161, L162, R163, S154, Y165, S166, F167, R168, P169, T170, Y171, G172, V173, G174, H175, Q176, P177, Y178, R179, V180, V181, V182, L183, S184, F185, E186, L187, L188, H189, A190, P191, A192, T193, V194, C195, G196, P197, K198, K199, S200, or T201 (amino acid numbering relative to SEQ ID NO: 1)).

In some embodiments, the amino acid sequence of the SARS-CoV-2 spike protein or polypeptide (e.g., a SARS-CoV-2 spike protein or polypeptide immunogen (or immunogenic fragment and/or immunogenic variant thereof)) comprises or consists of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 50 or more of the amino acid substitutions set forth in Table 2. In some embodiments, the amino acid sequence of the SARS-CoV-2 spike protein or polypeptide (e.g., a SARS-CoV-2 spike protein or polypeptide immunogen (or immunogenic fragment and/or immunogenic variant thereof)) comprises or consists of at least one, but no more than 2, 3, 4, 5, 6, 7, 8, 9, 10, 15, 20, 25, 30, 35, 40, or 50 of the amino acid substitutions set forth in Table 2. In some embodiments, the amino acid sequence of the SARS-CoV-2 spike protein or polypeptide (e.g., a SARS-CoV-2 spike protein or polypeptide immunogen (or immunogenic fragment and/or immunogenic variant thereof)) comprises or consists of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 50 or more of the amino acid substitutions set forth in Table 2. In some embodiments, the amino acid sequence of the SARS-CoV-2 spike protein or polypeptide (e.g., a SARS-CoV-2 spike protein or polypeptide immunogen (or immunogenic fragment and/or immunogenic variant thereof)) comprises or consists of from about 1-50, 1-40, 1-30, 1-20, 1-10, 1-9, 1-8, 1-7, 1-6, 1-5, 1-4, 1-3, or 1-2 of the amino acid substitutions set forth in Table 2.

In some embodiments, the amino acid sequence of the SARS-CoV-2 spike (e.g., a SARS-CoV-2 spike protein or polypeptide immunogen (or immunogenic fragment and/or immunogenic variant thereof)) comprises or consists of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 50 or more sets of amino acid substitutions set forth in Table 2. In some embodiments, the amino acid sequence of the SARS-CoV-2 spike protein or polypeptide (e.g., a SARS-CoV-2 spike protein or polypeptide immunogen (or immunogenic fragment and/or immunogenic variant thereof)) comprises or consists of at least one, but no more than 2, 3, 4, 5, 6, 7, 8, 9, 10, 15, 20, 25, 30, 35, 40, or 50 of the sets of amino acid substitutions set forth in Table 2. In some embodiments, the amino acid sequence of the SARS-CoV-2 spike protein or polypeptide (e.g., a SARS-CoV-2 spike protein or polypeptide immunogen (or immunogenic fragment and/or immunogenic variant thereof)) comprises or consists of 1, 2, 3, 4, 5, 6, 7, 8, 9, 10, 15, 20, 25, 30, 35, 40, 50 or more of the sets of amino acid substitutions set forth in Table 2. In some embodiments, the amino acid sequence of the SARS-CoV-2 spike protein or polypeptide (e.g., a SARS-CoV-2 spike protein or polypeptide immunogen (or immunogenic fragment and/or immunogenic variant thereof)) comprises or consists of from about 1-50, 1-40, 1-30, 1-20, 1-10, 1-9, 1-8, 1-7, 1-6, 1-5, 1-4, 1-3, or 1-2 of the sets of amino acid substitutions set forth in Table 2.

In some embodiments, the amino acid sequence of the SARS-CoV-2 spike protein or polypeptide (e.g., a SARS-CoV-2 spike protein or polypeptide immunogen (or immunogenic fragment and/or immunogenic variant thereof)) comprises or consists of one or more (e.g., 1, 2, 3, 4, 5, 6, 7, 8, 9, 10, 20, 30, 40, or 50 or more) amino acid variation that is not set forth in Table 2. In some embodiments, at least one of the one or more (e.g., 1, 2, 3, 4, 5, 6, 7, 8, 9, 10, 20, 30, 40, or 50 or more) amino acid variations is relative to the amino acid sequence of a reference SARS-CoV-2 spike protein (e.g., a naturally occurring SARS-CoV-2 spike protein). In some embodiments, at least one of the one or more (e.g., 1, 2, 3, 4, 5, 6, 7, 8, 9, 10, 20, 30, 40, or 50 or more) amino acid variations is relative to the amino acid sequence set forth in SEQ ID NO: 2. In some embodiments, at least one of the one or more (e.g., 1, 2, 3, 4, 5, 6, 7, 8, 9, 10, 20, 30, 40, or 50 or more) amino acid variations is relative to the amino acid sequence set forth in SEQ ID NO: 4. In some embodiments, at least one of the one or more (e.g., 1, 2, 3, 4, 5, 6, 7, 8, 9, 10, 20, 30, 40, or 50 or more) amino acid variations is found in one or more circulating variants of SARS-CoV-2. In some embodiments, at least one of the one or more (e.g., 1, 2, 3, 4, 5, 6, 7, 8, 9, 10, 20, 30, 40, or 50 or more) amino acid variations is found in one or more variants of SARS-CoV-2 that is known to have previously been circulating.

In some embodiments, the amino acid sequence of the SARS-CoV-2 spike protein or polypeptide (e.g., a SARS-CoV-2 spike protein or polypeptide immunogen (or immunogenic fragment and/or immunogenic variant thereof)) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids of the amino acid sequence of a naturally occurring SARS-CoV-2 spike protein. In some embodiments, outside of the inclusion of any amino acid substitution(s) (e.g., any set(s) of amino acid substitutions) set forth in Table 2, the amino acid sequence of the SARS-CoV-2 spike protein or polypeptide (e.g., a SARS-CoV-2 spike protein or polypeptide immunogen (or immunogenic fragment and/or immunogenic variant thereof)) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids of a naturally occurring SARS-CoV-2 spike protein.

In some embodiments, the amino acid sequence of the SARS-CoV-2 spike protein or polypeptide (e.g., a SARS-CoV-2 spike protein or polypeptide immunogen (or immunogenic fragment and/or immunogenic variant thereof)) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids of the amino acid sequence set forth in SEQ ID NO: 2. In some embodiments, outside of the inclusion of any amino acid substitution(s) (e.g., any set(s) of amino acid substitutions) set forth in Table 2, the amino acid sequence of the SARS-CoV-2 spike protein or polypeptide (e.g., a SARS-CoV-2 spike protein or polypeptide immunogen (or immunogenic fragment and/or immunogenic variant thereof)) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids of the amino acid sequence set forth in SEQ ID NO: 2.

In some embodiments, the amino acid sequence of the SARS-CoV-2 spike protein or polypeptide (e.g., a SARS-CoV-2 spike protein or polypeptide immunogen (or immunogenic fragment and/or immunogenic variant thereof)) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids of the amino acid sequence set forth in SEQ ID NO: 4. In some embodiments, outside of the inclusion of any amino acid substitution(s) (e.g., any set(s) of amino acid substitutions) set forth in Table 2, the amino acid sequence of the SARS-CoV-2 spike protein or polypeptide (e.g., a SARS-CoV-2 spike protein or polypeptide immunogen (or immunogenic fragment and/or immunogenic variant thereof)) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids of the amino acid sequence set forth in SEQ ID NO: 4.

In some embodiments, outside of the inclusion of any set(s) of amino acid substitutions set forth in Table 2, the amino acid sequence of the SARS-CoV-2 spike protein or polypeptide (e.g., a SARS-CoV-2 spike protein or polypeptide immunogen (or immunogenic fragment and/or immunogenic variant thereof)) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids of a naturally occurring SARS-CoV-2 spike protein. In some embodiments, outside of the inclusion of any set(s) of amino acid substitutions set forth in Table 2, the amino acid sequence of the SARS-CoV-2 spike protein or polypeptide (e.g., a SARS-CoV-2 spike protein or polypeptide immunogen (or immunogenic fragment and/or immunogenic variant thereof)) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids of the amino acid sequence set forth in SEQ ID NO: 2. In some embodiments, outside of the inclusion of any set(s) of amino acid substitutions set forth in Table 2, the amino acid sequence of the SARS-CoV-2 spike protein or polypeptide (e.g., a SARS-CoV-2 spike protein or polypeptide immunogen (or immunogenic fragment and/or immunogenic variant thereof)) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids of the amino acid sequence set forth in SEQ ID NO: 4.

In some embodiments, the SARS-CoV-2 spike protein or polypeptide (e.g., a SARS-CoV-2 spike protein or polypeptide immunogen (or immunogenic fragment and/or immunogenic variant thereof)) comprises at least about 10, 15, 20, 30, 40, 50, 60, 70, 80, 90, 100, 150, 200, 300, 400, 500, 600, 700, 800, 900, 1000, 1100, 1200, or 1300 amino acids. In some embodiments, the SARS-CoV-2 spike protein or polypeptide (e.g., a SARS-CoV-2 spike protein or polypeptide immunogen (or immunogenic fragment and/or immunogenic variant thereof)) comprises no more than about 10, 15, 20, 30, 40, 50, 60, 70, 80, 90, 100, 150, 200, 300, 400, 500, 600, 700, 800, 900, 1000, 1100, 1200, or 1300 amino acids. In some embodiments the SARS-CoV-2 spike protein or polypeptide (e.g., a SARS-CoV-2 spike protein or polypeptide immunogen (or immunogenic fragment and/or immunogenic variant thereof)) comprises from about 10-1300, 10-1200, 10-1100, 10-1000, 10-900, 10-800, 10-700, 10-600, 10-500, 10-400, 10-500, 10-400, 10-300, 10-250, 10-200, 10-100, 10-90, 10-80, 10-70, 10-60, 10-50, 10-40, 10-30, 10-20, 10-1300, 20-1300, 30-1300, 40-1300, 50-1300, 60-1300, 70-1300, 80-1300, 90-1300, 100-1300,10-250, 20-250, 30-250, 40-250, 50-250, 60-250, 70-250, 80-250, 90-250, or 100-250 amino acids. In some embodiments the SARS-CoV-2 spike protein or polypeptide (e.g., a SARS-CoV-2 spike protein or polypeptide immunogen (or immunogenic fragment and/or immunogenic variant thereof)) comprises from about 10-15, 10-20, 10-30, 10-40, 10-50, 10-60, 10-70, 10-80, 10-90, 10-100, 10-200, 10-300, 10-400, 10-500, 10-600, 10-700, 10-800, 10-900, 10-1000, 10-1100, 10-1200, or 10-1300 amino acids.

In some embodiments the SARS-CoV-2 spike protein or polypeptide (e.g., a SARS-CoV-2 spike protein or polypeptide immunogen (or immunogenic fragment and/or immunogenic variant thereof)) comprises from about 10-15, 10-20, 10-30, 10-40, 10-50, 10-60, 10-70, 10-80, 10-90, 10-100, 10-200, 10-300, 10-400, 10-500, 10-600, 10-700, 10-800, 10-900, 10-1000, 10-1100, 10-1200, 10-1300, 20-30, 20-40, 20-50, 20-60, 20-70, 20-80, 20-90, 20-100, 20-200, 20-300, 20-400, 20-500, 20-600, 20-700, 20-800, 20-900, 20-1000, 20-1100, 20-1200, 20-1300, 30-40, 30-50, 30-60, 30-70, 30-80, 30-90, 30-100, 30-200, 30-300, 30-400, 30-500, 30-600, 30-700, 30-800, 30-900, 30-1000, 30-1100, 30-1200, 30-1300, 40-50, 40-60, 40-70, 40-80, 40-90, 40-100, 40-200,40-300, 40-400,40-500, 40-600,40-700, 40-800,40-900, 40-1000,40-1100, 40-1200, 40-1300, 50-60, 50-70, 50-80, 50-90, 50-100, 50-200, 50-300, 50-500, 50-500, 50-600, 50-700, 50-800, 50-900, 50-1000, 50-1100, 50-1200, 50-1300, 60-70, 60-80, 60-90, 60-100, 60-200, 60-300, 60-400, 60-500, 60-600, 60-700, 60-800, 60-900, 60-1000, 60-1100, 60-1200,60-1300, 70-80, 70-90, 70-100, 70-200, 70-300, 70-400, 70-500, 70-600, 70-700, 70-800, 70-900, 70-1000, 70-1100, 70-1200, 70-1300, 80-90, 80-100, 80-200, 80-300, 80-400, 80-500, 80-600, 80-700, 80-800, 80-900, 80-1000, 80-1100, 80-1200, 80-1300, 90-100, 90-200, 90-300, 90-400, 90-500, 90-600, 90-700, 90-800, 90-900, 90-1000, 90-1100, 90-1200, 90-1300, 100-200, 100-300, 100-400, 100-500, 100-600, 100-700, 100-800, 100-900, 100-1000, 100-1100, 100-1200, 100-1300, 200-300, 200-400, 200-500, 200-600, 200-700, 200-800, 200-900, 200-1000, 200-1100, 200-1200, 200-1300, 300-400, 300-500, 300-600, 300-700, 300-800, 300-900, 300-1000, 300-1100, 300-1200, 300-1300, 400-500, 400-600, 400-700, 400-800, 400-900, 400-1000, 400-1100, 400-1200, 400-1300, 500-600, 500-700, 500-800, 500-900, 500-1000, 500-1100, 500-1200, 500-1300, 600-50, 600-60, 600-70, 600-80, 600-90, 600-100, 600-200, 600-300, 600-6000, 600-500, 600-600, 600-700, 600-800, 600-900, 600-1000, 600-1100, 600-1200, 600-1300, 700-50, 700-60, 700-70, 700-80, 700-90, 700-100, 700-200, 700-300, 700-7000, 700-500, 700-600, 700-700, 700-800, 700-900, 700-1000, 700-1100, 700-1200, 700-1300, 800-900, 800-1000, 800-1100, 800-1200, 800-1300,

900-50, 900-60, 900-70, 900-80, 900-90, 900-100, 900-200, 900-300, 900-9000, 900-500, 900-600, 900-700, 900-800, 900-900, 900-1000, 900-1100, 900-1200, 900-1300, 1000-1100, 1000-1200, 1000-1300, 1100-1200, 1100-1300, or 1200-1300.

In some embodiments, the SARS-CoV-2 spike protein or polypeptide (e.g., a SARS-CoV-2 spike protein or polypeptide immunogen (or immunogenic fragment and/or immunogenic variant thereof)) comprises at least a portion of the RBD of the SARS-CoV-2 spike protein. In some embodiments, the SARS-CoV-2 spike protein or polypeptide (e.g., a SARS-CoV-2 spike protein or polypeptide immunogen (or immunogenic fragment and/or immunogenic variant thereof)) comprises the RBD of the SARS-CoV-2 spike protein. In some embodiments, the SARS-CoV-2 spike protein or polypeptide (e.g., a SARS-CoV-2 spike protein or polypeptide immunogen (or immunogenic fragment and/or immunogenic variant thereof)) comprises a full-length SARS-CoV-2 spike protein. In some embodiments, the SARS-CoV-2 spike protein or polypeptide (e.g., a SARS-CoV-2 spike protein or polypeptide immunogen (or immunogenic fragment and/or immunogenic variant thereof)) comprises the ectodomain of the SARS-CoV-2 spike protein. In some embodiments, the SARS-CoV-2 spike protein or polypeptide (e.g., a SARS-CoV-2 spike protein or polypeptide immunogen (or immunogenic fragment and/or immunogenic variant thereof)) comprises the ectodomain of the SARS-CoV-2 spike protein and does not include the transmembrane domain or the cytoplasmic domain of the SARS-CoV-2 spike protein. In some embodiments, the SARS-CoV-2 spike protein or polypeptide (e.g., a SARS-CoV-2 spike protein or polypeptide immunogen (or immunogenic fragment and/or immunogenic variant thereof)) comprises a homologous signal peptide. In some embodiments, the SARS-CoV-2 spike protein or polypeptide (e.g., a SARS-CoV-2 spike protein or polypeptide immunogen (or immunogenic fragment and/or immunogenic variant thereof)) comprises a heterologous signal peptide. In some embodiments, the SARS-CoV-2 spike protein or polypeptide (e.g., a SARS-CoV-2 spike protein or polypeptide immunogen (or immunogenic fragment and/or immunogenic variant thereof)) does not contain a signal peptide.

In some embodiments, the SARS-CoV-2 spike protein or polypeptide (e.g., a SARS-CoV-2 spike protein or polypeptide immunogen (or immunogenic fragment and/or immunogenic variant thereof)) is stabilized in a prefusion state. In some embodiments, the SARS-CoV-2 spike protein or polypeptide (e.g., a SARS-CoV-2 spike protein or polypeptide immunogen (or immunogenic fragment and/or immunogenic variant thereof)) comprises at least one amino acid variation that stabilizes the SARS-CoV-2 spike protein or polypeptide (e.g., a SARS-CoV-2 spike protein or polypeptide immunogen (or immunogenic fragment and/or immunogenic variant thereof)) in a prefusion state (e.g., relative to a naturally occurring SARS-CoV-2 spike protein or polypeptide, relative to the amino acid sequence set forth in SEQ ID NO: 2). In some embodiments, the SARS-CoV-2 spike protein or polypeptide (e.g., a SARS-CoV-2 spike protein or polypeptide immunogen (or immunogenic fragment and/or immunogenic variant thereof)) comprises a proline at amino acid position 983 and/or a proline at amino acid position 984, amino acid numbering amino acid numbering relative to SEQ ID NO: 2. In some embodiments, the SARS-CoV-2 spike protein or polypeptide (e.g., a SARS-CoV-2 spike protein or polypeptide immunogen (or immunogenic fragment and/or immunogenic variant thereof)) comprises a proline at amino acid position 986 and/or a proline at amino acid position 987, amino acid numbering amino acid numbering relative to SEQ ID NO: 4.

In some embodiments, the SARS-CoV-2 spike protein or polypeptide (e.g., a SARS-CoV-2 spike protein or polypeptide immunogen (or immunogenic fragment and/or immunogenic variant thereof)) comprises an inactive furin cleavage site. In order to be able to trigger fusion, the spike protein of SARS-Cov-2 has to be cleaved into the S1 and S2 subunit. The cleavage site in SARS-Cov-2 is a polybasic motif (RRAR) (for example, amino acids 679-682 of SEQ ID NO: 2 or amino acids 682-685 of SEQ ID NO: 4 that can be activated by furin-like proteases). Modifications to the furin cleavage site that inactivate it are known in the art. See, e.g., Amanat F, Strohmeier S, Rathnasinghe R, et al. Introduction of two prolines and removal of the polybasic cleavage site leads to optimal efficacy of a recombinant spike-based SARS-CoV-2 vaccine in the mouse model. Preprint. bioRxiv. 2020; 2020.09.16.300970. Published 2020 Sep. 17. doi:10.1101/2020.09.16.300970 (describes the replacement of the RRAR cleavage site with a single alanine); and WO2022203963 (which describes the replacement of the RRAR cleavage site with the amino acid sequence QQAQ), the entire contents of each of which are incorporated herein by reference. In some embodiments, the SARS-CoV-2 spike protein or polypeptide (e.g., a SARS-CoV-2 spike protein or polypeptide immunogen (or immunogenic fragment and/or immunogenic variant thereof)) comprises at least one amino acid variation (e.g., relative to a naturally occurring SARS-CoV-2 spike protein or polypeptide, relative to the amino acid sequence set forth in SEQ ID NO: 2) in the furin cleavage site that inactivates the furin cleavage site. In some embodiments, the SARS-CoV-2 spike protein or polypeptide (e.g., a SARS-CoV-2 spike protein or polypeptide immunogen (or immunogenic fragment and/or immunogenic variant thereof)) comprises a replacement of the RRAR cleavage site with a single alanine. In some embodiments, the SARS-CoV-2 spike protein or polypeptide (e.g., a SARS-CoV-2 spike protein or polypeptide immunogen (or immunogenic fragment and/or immunogenic variant thereof)) comprises a replacement of the RRAR cleavage site with the amino acid sequence QQAQ.

In some embodiments, the SARS-CoV-2 spike protein or polypeptide (e.g., a SARS-CoV-2 spike protein or polypeptide immunogen (or immunogenic fragment and/or immunogenic variant thereof)) comprises one or more non-naturally occurring glycosylation motifs (e.g., N-glycosylation motifs). See, e.g., Lin Wei-Shuo et al., Glycan Masking of Epitopes in the NTD and RBD of the Spike Protein Elicits Broadly Neutralizing Antibodies Against SARS-CoV-2 Variants, Frontiers in Immunology, (12) Article 795741, 2 Dec. 2021, DOI=10.3389/fimmu.2021.795741, the entire contents of which are incorporated herein by reference for all purposes. In some embodiments, the inclusion of one or more glycosylation motif (e.g., N-glycosylation motif) facilitates glycan masking of an immunodominant epitope of the immunogenic protein (or immunogenic fragment or immunogenic variant thereof). In some embodiments, when the SARS-CoV-2 spike protein or polypeptide (e.g., a SARS-CoV-2 spike protein or polypeptide immunogen (or a SARS-CoV-2 spike protein or polypeptide immunogen fragment and/or immunogenic variant thereof)) is administered to a subject, the subject does not generate an effective amount of neutralizing antibodies that specifically bind to the immunodominant epitope. Thus, in some embodiments, the inclusion of one or more N-glycosylation motifs in the SARS-CoV-2 spike protein or polypeptide (e.g., a SARS-CoV-2 spike protein or polypeptide immunogen (or immunogenic fragment and/or immunogenic variant thereof)) shifts an immune response generated from the administration of the SARS-CoV-2 spike protein or polypeptide (e.g., a SARS-CoV-2 spike protein or polypeptide immunogen (or immunogenic fragment and/or immunogenic variant thereof)) to produce more neutralizing antibodies against the SARS-CoV-2 spike protein. Glycosylation motifs are known in the art. For example, NX1X2, wherein X1 can be any amino acid except for proline, and X2 can be S, T, or C, is known as a consensus N-glycosylation sequence.

In some embodiments, the SARS-CoV-2 spike protein or polypeptide (e.g., a SARS-CoV-2 spike protein or polypeptide immunogen (or immunogenic fragment and/or immunogenic variant thereof)) comprises one or more heterologous peptide or protein element, or the nucleic acid molecules described herein encode at least one heterologous peptide or protein element. In some embodiments, the at least one heterologous peptide or protein element may impart an additional function to the SARS-CoV-2 spike protein or polypeptide (e.g., a SARS-CoV-2 spike protein or polypeptide immunogen (or immunogenic fragment and/or immunogenic variant thereof)), e.g., to promote or improve secretion of the encoded SARS-CoV-2 spike protein or polypeptide (e.g., a SARS-CoV-2 spike protein or polypeptide immunogen (or immunogenic fragment and/or immunogenic variant thereof)) (e.g., via secretory signal peptides), promote or improve anchoring of the encoded the SARS-CoV-2 spike protein or polypeptide (e.g., a SARS-CoV-2 spike protein or polypeptide immunogen (or immunogenic fragment and/or immunogenic variant thereof)) described herein in the plasma membrane (e.g., via transmembrane elements), promote or improve formation of antigen complexes (e.g., via multimerization domains or antigen clustering elements), or promote or improve virus-like particle formation (VLP forming sequence). In some embodiments, the ectodomain of the SARS-CoV-2 spike protein or polypeptide (e.g., a SARS-CoV-2 spike protein or polypeptide immunogen (or immunogenic fragment and/or immunogenic variant thereof)) is modified to improve stability of the protein or polypeptide produced.

### 5.3 Nucleic Acid Molecules Encoding SARS-CoV-2 Spike Proteins (e.g., Immunogens)

In one aspect, provided herein are nucleic acid molecules comprising a coding region encoding a SARS-CoV-2 spike protein or polypeptide (e.g., a SARS-CoV-2 spike protein or polypeptide immunogen (or immunogenic fragment and/or immunogenic variant thereof)) described herein (see, e.g., § 5.2). In some embodiments, the nucleic acid molecule is RNA (e.g., mRNA or circular RNA) or DNA. In some embodiments, the nucleic acid (e.g., RNA) molecule is a translatable RNA. In some embodiments, the nucleic acid (e.g., RNA) molecule is a circular RNA. In some embodiments, the nucleic acid (e.g., RNA) molecule is mRNA.

In some embodiments, the nucleic acid molecule encoding the SARS-CoV-2 spike protein or polypeptide (e.g., a SARS-CoV-2 spike protein or polypeptide immunogen (or immunogenic fragment and/or immunogenic variant thereof)) (e.g., described herein) comprises from about 30 to about 20000 nucleotides, about 50 to about 20000 nucleotides, about 500 to about 10000 nucleotides, about 1000 to about 10000 nucleotides, about 1000 to about 5000 nucleotides, or about 2000 to about 5000 nucleotides. In some embodiments, the nucleic acid molecule encoding the SARS-CoV-2 spike protein or polypeptide (e.g., a SARS-CoV-2 spike protein or polypeptide immunogen (or immunogenic fragment and/or immunogenic variant thereof)) comprises at least 30 nucleotides, 50 nucleotides, 100 nucleotides, 200 nucleotides, 300 nucleotides, 400 nucleotides, 500 nucleotides, 1000 nucleotides, 2000 nucleotides, 3000 nucleotides, or 5000 nucleotides.

In some embodiments, the segment of the nucleic acid molecule encoding the SARS-CoV-2 spike protein or polypeptide (e.g., a SARS-CoV-2 spike protein or polypeptide immunogen (or immunogenic fragment and/or immunogenic variant thereof)) (e.g., described herein) comprises from about 30 to about 20000 nucleotides, about 50 to about 20000 nucleotides, about 500 to about 10000 nucleotides, about 1000 to about 10000 nucleotides, about 1000 to about 5000 nucleotides, or about 2000 to about 5000 nucleotides. In some embodiments, the segment of the nucleic acid molecule encoding the SARS-CoV-2 spike protein or polypeptide (e.g., a SARS-CoV-2 spike protein or polypeptide immunogen (or immunogenic fragment and/or immunogenic variant thereof)) comprises at least 30 nucleotides, 50 nucleotides, 100 nucleotides, 200 nucleotides, 300 nucleotides, 400 nucleotides, 500 nucleotides, 1000 nucleotides, 2000 nucleotides, 3000 nucleotides, or 5000 nucleotides.

In some embodiments, the nucleic acid molecule is altered (e.g., compared to the sequence of a reference nucleic acid molecule, a naturally occurring nucleic acid molecule), e.g., to impart one or more of (a) improved resistance to in vivo degradation, (b) improved stability in vivo, (c) reduced secondary structures, and/or (d) improved translatability in vivo, compared to the reference nucleic acid sequence. Alterations include, without limitation, e.g., codon optimization, nucleotide modifications (see, e.g., described herein), etc.

In some embodiments, the sequence of the nucleic acid molecule is codon optimized, e.g., for expression in humans. Codon optimization, in some embodiments, may be used to match codon frequencies in target and host organisms to ensure proper folding; bias guanosine (G) and/or cytosine (C) content to increase nucleic acid stability; minimize tandem repeat codons or base runs that may impair gene construction or expression; customize transcriptional and translational control regions; insert or remove protein trafficking sequences; remove/add post translation alteration sites in an encoded protein (e.g., glycosylation sites); add, remove, or shuffle protein domains; insert or delete restriction sites; modify ribosome binding sites and mRNA degradation sites; adjust translational rates to allow the various domains of a protein to fold properly; and/or to reduce or eliminate secondary structures (e.g., problem secondary structures) within the nucleic acid molecule. In some embodiments, the codon optimized nucleic acid sequence shows one or more of the above (compared to a reference nucleic acid sequence). In some embodiments, the codon optimized nucleic acid sequence shows one or more of improved resistance to in vivo degradation, improved stability in vivo, reduced secondary structures, and/or improved translatability in vivo, compared to a reference nucleic acid sequence. Codon optimization methods, tools, algorithms, and services are known in the art, non-limiting examples include services from GeneArt (Life Technologies) and DNA2.0 (Menlo Park Calif.). In some embodiments, the open reading frame (ORF) sequence is optimized using optimization algorithms (e.g., optimization algorithms known in the art). In some embodiments, the nucleic acid sequence is modified to optimize the number of G and/or C nucleotides as compared to a reference nucleic acid sequence. An increase in the number of G and C nucleotides may be generated by substitution of codons containing adenosine (A) or thymidine (T) (or uracil (U)) nucleotides by codons containing G or C nucleotides.

### 5.3.1 DNA Nucleic Acids

In some embodiments, the nucleic acid molecule is DNA. In some embodiments, the DNA is a linear coding DNA construct. In some embodiments, the DNA contained within a vector (e.g., a non-viral vector (e.g., a plasmid) or a viral vector). In some embodiments, the DNA is contained within a non-viral vector (e.g., a plasmid). In some embodiments, the DNA is contained within a viral vector (e.g., described herein). A more detailed description of vectors for both RNA and DNA nucleic acids is provided in § 5.6.

The coding DNA molecule may also comprise one or more heterologous nucleic acid elements to mediate expression of the coding region. These include, e.g., promoter(s), enhancer(s), polyadenylation signal(s), synthetic introns, transcriptional termination signals, polyadenylation sequences, and other transcription regulatory elements. A person of ordinary skill in the art is familiar with the transcriptional regulatory elements needed for expression of coding DNA can optimize the expression construct (e.g., linear DNA, plasmid DNA, etc.) accordingly.

In some embodiments, a promoter is operably connected to the respective coding nucleic acid sequence. The person of ordinary skill in the art is aware of various promoters that can be employed, for example, a promoter from simian virus 40 (SV40), a mouse mammary tumor virus (MMTV) promoter, a human immunodeficiency virus (HIV) promoter, bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter can also be a promoter from a human gene, for example, from human actin, human myosin, human hemoglobin, human muscle creatine, or human metalothionein. The promoter can also be a tissue specific promoter, such as a muscle or skin specific promoter, natural or synthetic. Examples of such promoters are described in US Patent Application Publication No. US20040175727, the entire contents of which is incorporated by reference herein for all purposes. Exemplary polyadenylation signals, include, but are not limited, to the bovine growth hormone (BGH) polyadenylation site, SV40 polyadenylation signals, and LTR polyadenylation signals.

### 5.3.2 RNA Nucleic Acids

In some embodiments, the nucleic acid molecule is an RNA molecule. In some embodiments, the RNA molecule is a translatable RNA molecule. In some embodiments, the RNA molecule is selected from an mRNA, a self-replicating RNA, a circular RNA, a viral RNA, or a replicon RNA. In some embodiments, the RNA molecule a circular RNA.

In some embodiments, the RNA molecule is a mRNA. The basic components of an mRNA molecule typically include at least one coding region (herein a coding region encoding at least one peptide or protein (e.g., a SARS-CoV-2 protein (e.g., a SARS-CoV-2 spike protein or polypeptide immunogen (or an immunogenic fragment and/or variant thereof)) (e.g., described herein), a 5′-untranslated region (UTR), a 3′-UTR, a 5′ cap, and a poly-A tail.

In some embodiments, the RNA (e.g., mRNA) comprises at least one heterologous UTR. The UTRs may harbor regulatory sequence elements that determine the RNA (e.g., mRNA) turnover, stability, localization, and/or expression of operably connected coding sequence(s). The heterologous UTRs may be derived from a naturally occurring gene or may be synthetically engineered. In some embodiments, the 5′-UTR comprises elements for controlling gene expression, e.g., ribosomal binding sites, miRNA binding sites. The 5′-UTR may be post-transcriptionally modified, e.g., by enzymatic or post-transcriptional addition of a 5′cap structure. In some embodiments, the 3′-UTR comprises a polyadenylation signal. In some embodiments, the RNA (e.g., mRNA) comprises at least one coding region encoding the polypeptide or protein (e.g., a SARS-CoV-2 protein (e.g., a SARS-CoV-2 spike protein or polypeptide immunogen (or an immunogenic fragment and/or immunogenic variant thereof)) (e.g., described herein) and 5′-UTR and/or a 3′-UTR. In some embodiments, the RNA (e.g., mRNA) comprises at least one coding sequence encoding a polypeptide or protein (e.g., a SARS-CoV-2 protein (e.g., a SARS-CoV-2 spike protein or polypeptide immunogen (or an immunogenic fragment and/or immunogenic variant thereof)) (e.g., described herein) operably connected to at least one heterologous 5′-UTR and at least one 3′-UTR.

In some embodiments, the RNA (e.g., mRNA) comprises a poly(A) sequence. The poly(A) sequence may comprise from about 10 to 500 adenosine nucleotides, 10 to 200 adenosine nucleotides, 20 to 200 adenosine nucleotides, 30 to 200 adenosine nucleotides, 40 to 200 adenosine nucleotides, or 50 to 200 adenosine nucleotides. In some embodiments, poly(A) sequence comprises at least 10, 20, 30, 40, 50, 60, 70, 80, 90, 100, 200, 300, 400, or 500 adenosine nucleotides. The poly(A) sequence may comprise from about 10 to 500 adenosine nucleotides, 10 to 200 adenosine nucleotides, 20 to 200 adenosine nucleotides, 30 to 200 adenosine nucleotides, 40 to 200 adenosine nucleotides, or 50 to 200 adenosine nucleotides, wherein the 3′ terminal nucleotide of said nucleic acid molecule is an adenosine. In some embodiments, poly(A) sequence comprises at least 10, 20, 30, 40, 50, 60, 70, 80, 90, 100, 200, 300, 400, or 500 adenosine nucleotides, wherein the 3′ terminal nucleotide of said nucleic acid molecule is an adenosine.

In some embodiments, the RNA (e.g., mRNA) comprises a 5′-cap structure. In some embodiments, the 5′-cap structure stabilizes the RNA (e.g., mRNA), enhances expression of the encoded polypeptide or protein (e.g., a SARS-CoV-2 protein (e.g., a SARS-CoV-2 spike protein or polypeptide immunogen (or an immunogenic fragment and/or immunogenic variant thereof)) (e.g., described herein) and/or reduces the stimulation of the innate immune system (e.g., after administration to a subject).

Exemplary 5′-cap structures include, but are not limited to, cap0 (methylation of the first nucleobase, e.g., m7GpppN), cap1 (additional methylation of the ribose of the adjacent nucleotide of m7GpppN), cap2 (additional methylation of the ribose of the 2nd nucleotide downstream of the m7GpppN), cap3 (additional methylation of the ribose of the 3rd nucleotide downstream of the m7GpppN), cap4 (additional methylation of the ribose of the 4th nucleotide downstream of the m7GpppN), ARCA (anti-reverse cap analogue), modified ARCA (e.g., phosphothioate modified ARCA), inosine, N1-methyi-guanosine, 2′-fluoro-guanosine, 7-deaza-guanosine, 8-oxo-guanosine, 2-amino-guanosine, LNA-guanosine, and 2-azido-guanosine. In some embodiments, the 5′ cap structure comprises m7G, cap0, cap1, cap2, a modified capO, or a modified cap1 structure.

In some embodiments, the RNA (e.g., mRNA) comprises one or more modified nucleotide (e.g., nucleotide analogue, backbone modifications, sugar modifications, and/or base modifications). A backbone modification in the context of the present disclosure is a modification, in which phosphates of the backbone of the nucleotides of the RNA (e.g., mRNA) are chemically modified. A sugar modification in the context of the present disclosure is a chemical modification of the sugar of the nucleotides of the RNA (e.g., mRNA). A base modification in the context of the present disclosure is a chemical modification of the base moiety of the nucleotides of the RNA (e.g., mRNA).

In some embodiments, the RNA (e.g., mRNA) comprises at least one modified nucleotide. Exemplary nucleotide analogues/modifications include, but are not limited to, 2-amino-6-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-lodo-2′-deoxycytidine-5′-triphosphate, 5-iodouridine-5′-triphosphate, 5-lodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-Propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thiocytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, 5′-O-(1-thiophosphate)-adenosine, 5′-O-(1-thiophosphate)-cytidine, 5′-O-(1-thiophosphate)-guanosine, 5′-O-(1-thiophosphatej-uridine, 5′-O-(1-thiophosphate)-pseudouridine, 6-aza-cytidine, 2-thio-cytidine, alpha-thio-cytidine, Pseudoiso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, Pyrrolo-cytidine, inosine, alpha-thioguanosine, 6-methyl-guanosine, 5-methyl-cytdine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, alpha-thioadenosine, 8-azido-adenosine, and 7-deaza-adenosine.

In some embodiments, the RNA (e.g., mRNA) comprises pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and/or 2′-O-methyl uridine.

In some embodiments, the RNA (e.g., mRNA) comprises one or more pseudouridine (ψ), N 1-methylpseudouridine (m1ψ), 5-methylcytosine, and 5-methoxyuridine. In some embodiments, essentially all, e.g., essentially 100% of the uracil in the coding sequence of the RNA (e.g., mRNA) have a chemical modification, preferably a chemical modification is in the 5-position of the uracil. Incorporating modified nucleotides such as e.g., pseudouridine (ψ), N1-methylpseudouridine (m1ψ), 5-methylcytosine, and/or 5-methoxyuridine into the coding sequence may be advantageous as unwanted innate immune responses (upon administration of the coding RNA or the vaccine) may be adjusted or reduced (if required).

In one embodiment, the RNA (e.g., mRNA) comprises: (i) a 5′-cap structure; (ii) a 5′-UTR; (iii) N1-methyl-pseudouridine, cytosine, adenine, and guanine; (iv) a 3′-UTR; and (v) a poly-A region.

RNA (e.g., mRNA) described herein can be generated by e.g., in vitro transcription. In vitro transcription is a method well known to those of ordinary skill in the art for the production of RNA (e.g., mRNA). Generally, the RNA is obtained by DNA-dependent in vitro transcription of an appropriate DNA template, e.g., a linearized plasmid DNA template or a PCR-amplified DNA template. The promoter for controlling RNA in vitro transcription can be any promoter for any DNA-dependent RNA polymerase. Examples of DNA-dependent RNA polymerases include the 17, T3, SP6, or Syn5 RNA polymerases. In some instances, the DNA template is linearized with a suitable restriction enzyme before it is subjected to RNA in vitro transcription. Reagents used in RNA in vitro transcription typically include: a DNA template (linearized plasmid DNA or PCR product) with a promoter sequence that has a high binding affinity for its respective RNA polymerase such as bacteriophage-encoded RNA polymerases (T7, T3, SP6, or Syn5); ribonucleotide triphosphates (NTPs) for the four bases (adenine, cytosine, guanine and uracil); a DNA-dependent RNA polymerase capable of binding to the promoter sequence within the DNA template (e.g., T7, T3, SP6, or Syn5 RNA polymerase); optionally, a ribonuclease (RNase) inhibitor to inactivate any potentially contaminating RNase; optionally, a pyrophosphatase to degrade pyrophosphate, which may inhibit RNA in vitro transcription; MgCh, which supplies Mg2+ ions as a co-factor for the polymerase; a buffer (TRIS or HEPES) to maintain a suitable pH value, which can also contain antioxidants (e.g., DTT), and/or polyamines such as spermidine at optimal concentrations, e.g., a buffer system comprising TRIS-Citrate as disclosed in WO2017109161. The obtained RNA (e.g., mRNA) products can be purified according to methods known in the art. For example, using PureMessenger® (CureVac, Tubingen, Germany; RP-HPLC according to WO2008077592) and/or tangential flow filtration (as described in WO2016193206) and/or oligo d(T) purification (see WO2016180430); or using RP-HPLC, e.g., using Reversed-Phase High pressure liquid chromatography (RP-HPLC), the entire contents of each reference is incorporated by reference herein for all purposes.

### 5.4 Fusions & Conjugates

In some embodiments, the SARS-CoV-2 spike protein or polypeptide (e.g., a SARS-CoV-2 spike protein or polypeptide immunogen (or immunogenic fragment and/or immunogenic variant thereof)) described herein (see, e.g., § 5.2) or the nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)) described herein (see, e.g., § 5.3) is operably connected to a heterologous moiety (e.g., a heterologous polypeptide) forming a fusion protein or polypeptide or conjugate.

In some embodiments, the heterologous moiety is a half-life extension moiety. Exemplary half-life extension moieties include, but are not limited to, a human immunoglobulin (hIg), a fragment of a hIg, a hIg constant region, a fragment of a hIg constant region, a hIg Fc region, human transferrin, human serum albumin (HSA), an HSA binding protein, and polyethylene glycol (PEG) (and polymers thereof). In some embodiments, the heterologous polypeptide is a half-life extension polypeptide. Exemplary half-life extension polypeptides include, but are not limited to, a hIg, a fragment of a hIg, one or more hIg heavy chain constant region, a fragment of a hIg constant region, a hIg Fc region, human transferrin, human serum albumin (HSA), and an HSA binding protein. The SARS-CoV-2 spike protein or polypeptide (e.g., a SARS-CoV-2 spike protein or polypeptide immunogen (or immunogenic fragment and/or immunogenic variant thereof)) or a nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)) fused or conjugated to a half-life extending moiety (e.g., a half-life extension polypeptide) can be evaluated for their pharmacokinetic properties utilizing standard in vivo methods known in the art. In some embodiments, the heterologous moiety is a detectable agent (e.g., protein, e.g., a fluorescent protein).

The heterologous moiety (e.g., heterologous polypeptide) can be directly operably connected or indirectly operably connected to the SARS-CoV-2 spike protein or polypeptide (e.g., a SARS-CoV-2 spike protein or polypeptide immunogen (or immunogenic fragment and/or immunogenic variant thereof)) or the nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)).

In some embodiments, the heterologous moiety is directly operably connected to the SARS-CoV-2 spike protein or polypeptide (e.g., a SARS-CoV-2 spike protein or polypeptide immunogen (or immunogenic fragment and/or immunogenic variant thereof)) or the nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)). In some embodiments, the heterologous moiety is indirectly operably connected to the SARS-CoV-2 spike protein or polypeptide (e.g., a SARS-CoV-2 spike protein or polypeptide immunogen (or immunogenic fragment and/or immunogenic variant thereof)) or the nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)).

A person of ordinary skill in the art can evaluate and select a suitable linker for the fusion or conjugation of a specific heterologous moiety. For example, in embodiments, wherein the heterologous moiety comprises a heterologous polypeptide, a peptide linker may be employed. Peptide linkers are known in the art and can be selected based on specific properties, including e.g., length, flexibility, rigidity, cleavability, etc. The amino acid sequence of commonly employed peptide linkers comprises glycine amino acid residues, serine amino acid residues, glycine and serine amino acid residues, or glycine, serine, and proline amino acid residues.

The heterologous moiety (e.g., heterologous polypeptide) and the SARS-CoV-2 spike protein or polypeptide (e.g., a SARS-CoV-2 spike protein or polypeptide immunogen (or immunogenic fragment and/or immunogenic variant thereof)) or the nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)) can be arranged in orientation so long as each functional part of the fusion protein or polypeptide or conjugate maintains the ability to mediate its function.

### 5.5 Vaccine Compositions

In some embodiments, a SARS-CoV-2 spike protein or polypeptide (e.g., a SARS-CoV-2 spike protein or polypeptide immunogen (or immunogenic fragment and/or immunogenic variant thereof)) described herein (see, e.g., § 5.2) (or a fusion or conjugate thereof (see, e.g., § 5.4)) or a nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)) described herein (see, e.g., § 5.3) (or a fusion or conjugate thereof (see, e.g., § 5.4)) forms the basis for a vaccine composition (e.g., a prime vaccine composition, a prime boost composition, a vaccine prime and booster composition). Therefore, in one aspect, provided herein are vaccine compositions (e.g., prime vaccine compositions, prime boost compositions, vaccine prime and booster compositions) comprising at least one SARS-CoV-2 spike protein or polypeptide (e.g., a SARS-CoV-2 spike protein or polypeptide immunogen (or immunogenic fragment and/or immunogenic variant thereof)) described herein (see, e.g., § 5.2) (or a fusion or conjugate thereof (see, e.g., § 5.4)) or a nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)) described herein (see, e.g., § 5.3) (or a fusion or conjugate thereof (see, e.g., § 5.4)).

In some embodiments, the vaccine composition is a prime vaccine composition of a prime-boost vaccine regimen. In some embodiments, the vaccine composition is a prime boost composition of a prime-boost vaccine regimen. In some embodiments, the vaccine composition is a vaccine prime and booster composition of a prime-boost vaccine regimen. In some embodiments, the prime boost composition can be utilized as a prime and or a booster (e.g., as described herein). In some embodiments, the vaccine composition forms a single dose vaccine that does not require a booster.

### 5.5.1 Peptide and Protein-Based Vaccines

In some embodiments, a SARS-CoV-2 spike protein or polypeptide (e.g., a SARS-CoV-2 spike protein or polypeptide immunogen (or immunogenic fragment and/or immunogenic variant thereof)) described herein (see, e.g., § 5.2) (or a fusion or conjugate thereof (see, e.g., § 5.4)) forms the basis for a vaccine composition (e.g., a prime vaccine composition, a prime boost composition, a vaccine prime and booster composition). Therefore, in one aspect, provided herein are vaccine compositions (e.g., prime vaccine compositions, prime boost compositions, vaccine prime and booster compositions) comprising at least one SARS-CoV-2 spike protein or polypeptide (e.g., a SARS-CoV-2 spike protein or polypeptide immunogen (or immunogenic fragment and/or immunogenic variant thereof)) described herein (see, e.g., § 5.2) (or a fusion or conjugate thereof (see, e.g., § 5.4)).

In some embodiments, the vaccine composition is a prime vaccine composition of a prime-boost vaccine regimen. In some embodiments, the vaccine composition is a prime boost composition of a prime-boost vaccine regimen. In some embodiments, the vaccine composition is a vaccine prime and booster composition of a prime-boost vaccine regimen. In some embodiments, the prime boost composition can be utilized as a prime and or a booster (e.g., as described herein). In some embodiments, the vaccine composition forms a single dose vaccine that does not require a booster.

In some embodiments, the vaccine composition comprises a plurality of SARS-CoV-2 spike proteins or polypeptide (e.g., immunogens (or immunogenic fragments and/or immunogenic variants thereof)).

In some embodiments, the plurality comprises or consists of at least 2, 3, 4, 5, 6, 7, 8, 9, 10, 11, 12, 13, 14, 15, 16, 17, 18, 19, 20, 30, 40, 50, 60, 70, 80, 90, 100 or more SARS-CoV-2 spike proteins or polypeptides (e.g., immunogens (or immunogenic fragments and/or immunogenic variants thereof)). In some embodiments, the plurality comprises or consists of from about 2-100, 2-90, 2-80, 2-70, 2-60, 2-50, 2-40, 2-30, 2-20, 2-10, 2-5, 5-100, 5-90, 5-80, 5-70, 5-60, 5-50, 5-40, 5-30, 5-20, 5-10, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90,40-80, 40-70,40-60, 40-50, 50-100, 50-90, 50-80, 50-70, 50-60, 60-100, 60-90, 60-80, 60-70, 70-100, 70-90, 70-80, 80-100, 80-90, or 90-100 SARS-CoV-2 spike proteins or polypeptides (e.g., immunogens (or immunogenic fragments and/or immunogenic variants thereof)). In some embodiments, the plurality comprises at least 2 but no more than 100, 90, 80, 70, 60, 50, 40, 30, 20, 19, 18, 17, 16, 15, 14, 13, 12, 11, 10, 9, 8, 7, 6, 5, 4, 3 SARS-CoV-2 spike proteins or polypeptides (e.g., immunogens (or immunogenic fragments or immunogenic variants thereof)).

In some embodiments, the amino acid sequence of each of the SARS-CoV-2 spike proteins or polypeptides (e.g., immunogens (or immunogenic fragments and/or immunogenic variants thereof)) of the plurality is different.

In some embodiments, the amino acid sequence of at least one of the SARS-CoV-2 spike proteins or polypeptides (e.g., immunogens (or immunogenic fragments and/or immunogenic variants thereof)) of the plurality is derived from a circulating strain of SARS-CoV-2.

In some embodiments, the amino acid sequence of a first SARS-CoV-2 spike protein or polypeptide (e.g., a SARS-CoV-2 spike protein or polypeptide immunogen (or immunogenic fragment and/or immunogenic variant thereof)) of the plurality comprises at least a first amino acid substitution (e.g., a set of amino acid substitutions) set forth in Table 2; and the amino acid sequence of a second SARS-CoV-2 spike protein or polypeptide (e.g., a SARS-CoV-2 spike protein or polypeptide immunogen (or immunogenic fragment and/or immunogenic variant thereof)) of the plurality comprises at least a second amino acid substitution (e.g., a set of amino acid substitutions) set forth in Table 2, wherein the first and second amino acid substitutions (e.g., the first and second sets of amino acid substitutions) set forth in Table 2 are different. In some embodiments, the amino acid sequence of at least two (e.g., at least 3, 4, 5, 6, 7, 8, 9, 10, or more) of the SARS-CoV-2 spike proteins or polypeptides (e.g., immunogens (or immunogenic fragments and/or immunogenic variants thereof)) of the plurality contain at least one amino acid substitution (e.g., at least one set of amino acid substitutions) set forth in Table 2. In some embodiments, at least two (e.g., at least 3, 4, 5, 6, 7, 8, 9, 10, or more) of the SARS-CoV-2 spike proteins or polypeptides (e.g., immunogens (or immunogenic fragments and/or immunogenic variants thereof)) of the plurality contain at least one amino acid substitution (e.g., at least one set of amino acid substitutions) set forth in Table 2, wherein each amino acid substitution (e.g., each set of amino acid substitutions) is different.

In some embodiments, the amino acid sequence of at least one (e.g., at least 2, 3, 4, 5, 6, 7, 8, 9, 10, 20, 30, 40, or 50 or more) of the SARS-CoV-2 spike proteins or polypeptides (e.g., immunogens (or immunogenic fragments and/or immunogenic variants thereof)) of the plurality comprises or consists of one or more amino acid variation that is not set forth in Table 2. In some embodiments, the one or more amino acid variations are relative to the amino acid sequence of a reference SARS-CoV-2 spike protein or polypeptide (e.g., a naturally occurring SARS-CoV-2 spike protein). In some embodiments, the one or more amino acid variations are relative to the amino acid sequence set forth in SEQ ID NO: 2. In some embodiments, the one or more amino acid variations are relative to the amino acid sequence set forth in SEQ ID NO: 4. In some embodiments, the one or more variations are found in one or more circulating variants of SARS-CoV-2. In some embodiments, the one or more variations are found in one or more variant of SARS-CoV-2 that are known to have previously circulated.

In some embodiments, the vaccine composition further comprises at least one immunogen (or immunogenic fragment and/or immunogenic variant thereof) from a non-SARS-CoV-2 virus, e.g., an influenza virus (e.g., influenza A, influenza B), a respiratory syncytial virus (RSV), a rhinovirus, a parvovirus, a parainfluenza virus, an adenovirus. In some embodiments, vaccine composition comprises one or more immunogen (or immunogenic fragment or immunogenic variant thereof) from an influenza virus (e.g., influenza A, influenza B), a respiratory syncytial virus (RSV), a rhinovirus, a parvovirus, a parainfluenza virus, and/or an adenovirus (or any combination thereof).

In some embodiments, the SARS-CoV-2 spike protein or polypeptide (e.g., a SARS-CoV-2 spike protein or polypeptide immunogen (or immunogenic fragment and/or immunogenic variant thereof)) (e.g., ectodomain of the SARS-CoV-2 spike protein or polypeptide) is modified to improve expression of the protein in host cells (e.g., insect cells, mammalian cells, eggs) as described below.

Polypeptides and proteins (e.g., immunogens) described herein, e.g., SARS-CoV-2 spike proteins and polypeptides (e.g., immunogens (and immunogenic fragments and/or immunogenic variants thereof)) may be produced by recombinant technology in host cells (e.g., insect cells, mammalian cells, bacteria) that have been transfected or transduced with a nucleic acid expression vector (e.g., plasmid, viral vector (e.g., a baculoviral expression vector)) encoding the SARS-CoV-2 spike protein or polypeptide (e.g., the immunogen (or the immunogenic fragment and/or immunogenic variant thereof)). Such general methods are common knowledge in the art. The expression vector typically contains an expression cassette that includes nucleic acid sequences capable of bringing about expression of the nucleic acid molecule encoding the SARS-CoV-2 spike protein or polypeptide (e.g., the immunogen (or the immunogenic fragment and/or immunogenic variant thereof)), such as promoter(s), enhancer(s), polyadenylation signals, and the like. The person of ordinary skill in the art is aware that various promoter and enhancer elements can be used to obtain expression of a nucleic acid molecule in a host cell. For example, promoters can be constitutive or regulated, and can be obtained from various sources, e.g., viruses, prokaryotic or eukaryotic sources, or artificially designed. Post transfection or transduction, host cells containing the expression vector encoding the SARS-CoV-2 spike protein or polypeptide (e.g., the immunogen (or the immunogenic fragment and/or immunogenic variant thereof)) are cultured under conditions conducive to expression of the nucleic acid molecule encoding the SARS-CoV-2 spike protein or polypeptide (e.g., the immunogen (or the immunogenic fragment and/or immunogenic variant thereof)). Culture media is available from various vendors, and a suitable medium can be routinely chosen for a host cell to express a polypeptide or protein of interest, here the SARS-CoV-2 spike protein or polypeptide (e.g., the immunogen (or the immunogenic fragment and/or immunogenic variant thereof)). Host cells can be adherent or suspension cultures, and a person of ordinary skill in the art can optimize culture methods for specific host cells selected. For example, suspension cells can be cultured in, for example, bioreactors in e.g., a batch process or a fed-batch process. The produced immunogenic peptide or protein may be isolated from the cell cultures, by, for example, column chromatography in either flow-flow through or bind-and-elute modes. Examples include, but are not limited to, ion exchange resins and affinity resins, such as lentil lectin Sepharose, and mixed mode cation exchange-hydrophobic interaction columns (CEX-HIC). The peptide or protein may be concentrated, buffer exchanged by ultrafiltration, and the retentate from the ultrafiltration may be filtered through an appropriate filter, e.g., a 0.22 μm filter. See, e.g., McPherson et al., “Development of a SARS Coronavirus Vaccine from Recombinant Spike Protein Plus Delta Inulin Adjuvant,” Chapter 4, in Sunil Thomas (ed.), Vaccine Design: Methods and Protocols: Volume 1: Vaccines for Human Diseases, Methods in Molecular Biology, Springer, New York, 2016. See also U.S. Pat. No. 5,762,939, the entire contents of each of which is incorporated by reference herein for all purposes.

The SARS-CoV-2 spike proteins and polypeptides (e.g., immunogens (or immunogenic fragments and/or immunogenic variants thereof)) described herein may also be produced synthetically. The SARS-CoV-2 spike proteins and polypeptides (e.g., immunogens (or immunogenic fragments and/or immunogenic variants thereof)) described herein may be produced by using an egg-based manufacturing method.

In some embodiments, the SARS-CoV-2 spike proteins and polypeptides (e.g., immunogens (or immunogenic fragments and/or immunogenic variants thereof)) of the vaccine composition are formulated in one or more carrier (e.g., a carrier described herein (see, e.g., § 5.7)).

In some embodiments, the vaccine compositions are pharmaceutical compositions (e.g., described herein, e.g., see § 5.8). In some embodiments, the vaccine compositions comprise an adjuvant (e.g., described herein, e.g., see § 5.9).

### 5.5.2 Nucleic Acid-Based Vaccines

In some embodiments, a nucleic acid molecule comprising a coding region encoding a SARS-CoV-2 spike protein or polypeptide (e.g., a SARS-CoV-2 spike protein or polypeptide immunogen (or immunogenic fragment and/or immunogenic variant thereof)) described herein (see, e.g., § 5.3) (or a fusion or conjugate thereof (see, e.g., § 5.4)) forms the basis for a vaccine composition (e.g., a prime vaccine composition, a prime boost composition, a vaccine prime and booster composition). Therefore, in one aspect, provided herein are vaccine compositions (e.g., prime vaccine compositions, prime boost compositions, vaccine prime and booster compositions) comprising a nucleic acid molecule comprising a coding region encoding at least one SARS-CoV-2 spike protein or polypeptide (e.g., a SARS-CoV-2 spike protein or polypeptide immunogen (or immunogenic fragment and/or immunogenic variant thereof)) described herein (see, e.g., § 5.3) (or a fusion or conjugate thereof (see, e.g., § 5.4)).

In some embodiments, the vaccine composition is a prime vaccine composition of a prime-boost vaccine regimen. In some embodiments, the vaccine composition is a prime boost composition of a prime-boost vaccine regimen. In some embodiments, the vaccine composition is a vaccine prime and booster composition of a prime-boost vaccine regimen. In some embodiments, the prime boost composition can be utilized as a prime and or a booster (e.g., as described herein). In some embodiments, the vaccine composition forms a single dose vaccine that does not require a booster.

In some embodiments, the vaccine composition comprises a plurality of nucleic acid moles, each comprising a coding region encoding at least one SARS-CoV-2 spike protein or polypeptide (e.g., a SARS-CoV-2 spike protein or polypeptide immunogen (or immunogenic fragment and/or immunogenic variant thereof)) (e.g., described herein).

In some embodiments, each of the nucleic acid molecules of the plurality are part of the same larger nucleic acid molecule. In some embodiments, each of the nucleic acid molecules of the plurality are separate (i.e., not connected) nucleic acid molecules. In some embodiments, at least two of the nucleic acid molecules of the plurality are part of the same larger nucleic acid molecule. In some embodiments, at least two of the nucleic acid molecules of the plurality are separate (i.e., not connected) nucleic acid molecules. In some embodiments, at least two of the nucleic acid molecules of the plurality are part of the same larger nucleic acid molecule; and at least one (e.g., at least 2, 3, 4, 5, etc.) of the nucleic acid molecules of the plurality is a separate (i.e., not connected) nucleic acid molecule.

In some embodiments, the plurality comprises or consists of at least 2, 3, 4, 5, 6, 7, 8, 9, 10, 11, 12, 13, 14, 15, 16, 17, 18, 19, 20, 30, 40, 50, 60, 70, 80, 90, 100 or more nucleic acid molecules. In some embodiments, the plurality comprises or consists of from about 2-100, 2-90, 2-80, 2-70, 2-60, 2-50, 2-40, 2-30, 2-20, 2-10, 2-5, 5-100, 5-90, 5-80, 5-70, 5-60, 5-50, 5-40, 5-30, 5-20, 5-10, 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, or 90-100 nucleic acid molecules. In some embodiments, the plurality comprises at least 2 but no more than 100, 90, 80, 70, 60, 50, 40, 30, 20, 19, 18, 17, 16, 15, 14, 13, 12, 11, 10, 9, 8, 7, 6, 5, 4, 3 nucleic acid molecules.

In some embodiments, the amino acid sequence of each of the encoded SARS-CoV-2 spike proteins or polypeptides (e.g., immunogens (or immunogenic fragments and/or immunogenic variants thereof)) of the plurality is different.

In some embodiments, the amino acid sequence of at least one of the encoded SARS-CoV-2 spike proteins or polypeptides (e.g., immunogens (or immunogenic fragments and/or immunogenic variants thereof)) of the plurality is derived from a circulating strain of SARS-CoV-2.

In some embodiments, the amino acid sequence of a first encoded SARS-CoV-2 spike protein or polypeptide (e.g., a SARS-CoV-2 spike protein or polypeptide immunogen (or immunogenic fragment and/or immunogenic variant thereof)) of the plurality comprises at least a first amino acid substitution (e.g., a first set of amino acid substitutions) set forth in Table 2; and the amino acid sequence of a second encoded SARS-CoV-2 spike protein or polypeptide (e.g., a SARS-CoV-2 spike protein or polypeptide immunogen (or immunogenic fragment and/or immunogenic variant thereof)) of the plurality comprises at least a second amino acid substitution (e.g., a second set of amino acid substitutions) set forth in Table 2, wherein the first and second amino acid substitutions (e.g., the first and second sets of amino acid substitutions) set forth in Table 2 are different. In some embodiments, the amino acid sequence of at least two (e.g., at least 3, 4, 5, 6, 7, 8, 9, 10, or more) of the encoded SARS-CoV-2 spike proteins or polypeptides (e.g., immunogens (or immunogenic fragments and/or immunogenic variants thereof)) of the plurality contain at least one amino acid substitution (e.g., at least one set of amino acid substitutions) set forth in Table 2. In some embodiments, the amino acid sequence of at least two (e.g., at least 3, 4, 5, 6, 7, 8, 9, 10, or more) of the encoded SARS-CoV-2 spike proteins or polypeptides (e.g., immunogens (or immunogenic fragments and/or immunogenic variants thereof)) of the plurality contain an amino acid substitution (e.g., a set of amino acid substitutions) set forth in Table 2, wherein each amino acid substitution (e.g., each set of amino acid substitutions) is different.

In some embodiments, the amino acid sequence of at least one (e.g., at least 2, 3, 4, 5, 6, 7, 8, 9, 10, or more) of the encoded SARS-CoV-2 spike proteins or polypeptides (e.g., immunogens (or immunogenic fragments or immunogenic variants thereof)) of the plurality comprises or consists of one or more (e.g., 1, 2, 3, 4, 5, 6, 7, 8, 9, 10, 20, 30, 40, or 50, or more) amino acid variation that is not set forth in Table 2. In some embodiments, the one or more amino acid variations are relative to the amino acid sequence of a reference SARS-CoV-2 spike protein or polypeptide (e.g., a naturally occurring SARS-CoV-2 spike protein). In some embodiments, the one or more amino acid variations are relative to the amino acid sequence set forth in SEQ ID NO: 2. In some embodiments, the one or more amino acid variations are relative to the amino acid sequence set forth in SEQ ID NO: 4. In some embodiments, the one or more amino acid variations are found in one or more circulating variants of SARS-CoV-2. In some embodiments, the one or more amino acid variations are found in one or more SARS-CoV-2 strain that is known to have previously circulated.

In some embodiments, the vaccine composition further comprises one or more nucleic acid molecule encoding one or more immunogen (e.g., one or more immunogenic peptide or protein) from a non-SARS-CoV-2 virus, e.g., an influenza virus (e.g., influenza A, influenza B), a respiratory syncytial virus (RSV), a rhinovirus, a parvovirus, a parainfluenza virus, or an adenovirus. In some embodiments, vaccine composition comprises one or more nucleic acid molecule encoding one or more immunogen (e.g., one or more immunogenic peptide or protein) from an influenza virus (e.g., influenza A, influenza B), a respiratory syncytial virus (RSV), a rhinovirus, a parvovirus, a parainfluenza virus, and/or an adenovirus (or any combination thereof).

In some embodiments, the nucleic acid molecules are comprised within one or more vectors (e.g., vectors described herein (see, e.g., § 5.6). In some embodiments, the nucleic acid molecules or the vectors of the vaccine composition are formulated in one or more carrier (e.g., a carrier described herein (see, e.g., § 5.7).

In some embodiments, the vaccine compositions are pharmaceutical compositions (e.g., described herein, e.g., see § 5.8). In some embodiments, the vaccine compositions comprise an adjuvant (e.g., described herein, e.g., see § 5.9).

Nucleic acid molecules can be generated using common methods known in the art and described above in § 5.3.

### 5.6 Vectors

In some embodiments, the nucleic acid molecules described herein (e.g., DNA molecules, RNA molecules (e.g., mRNA molecules)) (see, e.g., § 5.3) (or a fusion or conjugate thereof (see, e.g., § 5.4)) are contained in a vector (e.g., a non-viral vector, a viral vector). Thus, in one aspect, also provided herein are vectors (e.g., viral vectors, non-viral vectors (e.g., plasmids, minicircles)) comprising one or more nucleic acid molecule described herein (e.g., nucleic acid molecules encoding a SARS-CoV-2 spike protein or polypeptide (e.g., a SARS-CoV-2 spike protein or polypeptide immunogen (or an immunogenic fragment and/or immunogenic variant thereof)). Such vectors can be easily manipulated by methods well known to the ordinary person of skill in the art.

In some embodiments, the vector is a non-viral vector (e.g., a plasmid, minicircle). In some embodiments, the vector is a plasmid. A person of ordinary skill in the art is aware of suitable plasmids for expression of the DNA of interest. For example, Suitable plasmid DNA may be generated to allow efficient production of the encoded peptides or proteins (e.g., SARS-CoV-2 proteins or polypeptides (e.g., immunogens) in cell lines, e.g., in insect cell lines, for example using vectors as described in WO2009150222A2 and as defined in PCT claims 1 to 33, the disclosure relating to claim 1 to 33 of WO2009150222A2 the entire contents of which is incorporated by reference herein for all purposes.

In some embodiments, the vector is a viral vector. Viral vectors include both RNA and DNA based vectors. The vectors can be designed to meet a variety of specifications. For example, viral vectors can be engineered to be capable or incapable of replication in prokaryotic and/or eukaryotic cells. In some embodiments, the vector is replication deficient. In some embodiments, the vector is replication competent. Viral vectors can be engineered or selected that either will (or will not) integrate in whole or in part into the genome of host cells, resulting (or not (e.g., episomal expression)) in stable host cells comprising the desired nucleic acid in their genome.

Exemplary viral vectors include, but are not limited to, adenovirus vectors, adeno-associated virus vectors, lentivirus vectors, retrovirus vectors, poxvirus vectors, parapoxivirus vectors, vaccinia virus vectors, fowlpox virus vectors, herpes virus vectors, adeno-associated virus vectors, alphavirus vectors, lentivirus vectors, rhabdovirus vectors, measles virus, Newcastle disease virus vectors, picornaviruses vectors, or lymphocytic choriomeningitis virus vectors. In some embodiments, the viral vector is an adenovirus vector, adeno-associated virus vector, or a lentivirus vector.

In some embodiments, the vector is an adenovirus vector (e.g., a human adenoviral vector, e.g., HAdV or AdHu). In some embodiments, the adenovirus vector has the E1 region deleted, rendering it replication-deficient in human cells. Other regions of the adenovirus such as E3 and E4 may also be deleted. Exemplary adenovirus vectors include, but are not limited to, those described in e.g., WO2005071093 or WQ2006048215, the entire contents of each of which is incorporated herein by reference for all purposes. In some embodiments, the adenovirus-based vector used is a simian adenovirus, thereby avoiding dampening of the immune response after vaccination by pre-existing antibodies to common human entities such as AdHu5. Exemplary, simian adenovirus vectors include AdCh63 (see, e.g., WO2005071093, the entire contents of which is incorporated herein by reference for all purposes) or AdCh68.

Viral vectors can be generated through the use of a packaging/producer cell line (e.g., a mammalian cell line) using standard methods known to the person of ordinary skill in the art. Generally, a nucleic acid construct (e.g., a plasmid) encoding the peptide or protein of interest (e.g., a peptide or protein described herein (e.g., SARS-CoV-2 peptide or protein described herein (e.g., a SARS-CoV-2 immunogen (or immunogenic fragment and/or immunogenic variant thereof)) (along with additional elements e.g., a promoter, inverted terminal repeats (ITRs) flanking the transgene, a plasmid encoding e.g., viral replication and structural proteins, along with one or more helper plasmids a host cell (e.g., a host cell line) are transfected into a host cell line (i.e., the packing/producer cell line). In some instances, depending on the viral vector, a helper plasmid may also be needed that include helper genes from another virus (e.g., in the instance of adeno-associated viral vectors). Eukaryotic expression plasmids are commercially available from a variety of suppliers, for example the plasmid series: pcDNA™, pCR3.1™, pCMV™, pFRT™ pVAX1™, pCI™, Nanoplasmid™, and Pcaggs. The person of ordinary skill in the art is aware of numerous transfection methods and any suitable method of transfection may be employed (e.g., using a biochemical substance as carrier (e.g., lipofectamine), by mechanical means, by electroporation). The cells are cultured under conditions suitable and for a sufficient time for plasmid expression. The viral particles may be purified from the cell culture medium using standard methods known to the person of ordinary skill in the art. For example, by centrifugation followed by e.g., chromatography and/or ultrafiltration.

### 5.7 Carriers

In some embodiments, a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule comprising a coding region encoding the SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), or a vector comprising a nucleic acid molecule comprising a coding region encoding the SARS-CoV-2 spike protein or polypeptide (e.g., a SARS-CoV-2 spike protein or polypeptide immunogen (or an immunogenic fragment and/or immunogenic variant thereof)) described herein is formulated within one or more carrier. Therefore, further provided herein are carriers comprising a SARS-CoV-2 spike protein or polypeptide (e.g., a SARS-CoV-2 spike protein or polypeptide immunogen (or an immunogenic fragment and/or immunogenic variant thereof)) described herein, a nucleic acid molecule comprising a coding region encoding a SARS-CoV-2 spike protein or polypeptide (e.g., a SARS-CoV-2 spike protein or polypeptide immunogen (or an immunogenic fragment and/or immunogenic variant thereof)) described herein, or a vector comprising a nucleic acid molecule comprising a coding region encoding the SARS-CoV-2 spike protein or polypeptide (e.g., a SARS-CoV-2 spike protein or polypeptide immunogen (or an immunogenic fragment and/or immunogenic variant thereof)) described herein.

Proteins, peptides, nucleic acid molecules (e.g., RNA (e.g., mRNA), DNA), and vectors, can be encapsulated within a carrier, chemically conjugated to a carrier, and/or associated with a carrier. In this context, the term “associated” refers to the essentially stable combination of a protein, peptide, nucleic acid molecule (e.g., RNA (e.g., mRNA, DNA), or vector with one or more molecules of a carrier (e.g., one or more lipids of a lipid-based carrier, e.g., a lipid nanoparticle (LNP), liposome, lipoplex, and/or nanoliposome) into larger complexes or assemblies without covalent binding. In this context, the term “encapsulation” refers to the incorporation of a protein, peptide, nucleic acid molecule (e.g., RNA (e.g., mRNA), DNA), or vector into a carrier (e.g., a lipid-based carrier, e.g., an LNP, liposome, lipoplex, and/or nanoliposome) wherein the protein, peptide, nucleic acid molecule, e.g., the RNA (e.g., mRNA, DNA), or vector is entirely contained within the interior space of the carrier (e.g., the lipid-based carrier, e.g., the LNP, liposome, lipoplex, and/or nanoliposome).

Exemplary carriers includes, but are not limited to, lipid-based carriers (e.g., LNPs, liposomes, lipoplexes, and nanoliposomes). In some embodiments, the carrier is a lipid-based carrier. In some embodiments, the carrier is an LNP. In some embodiments, the LNP comprises a cationic lipid, a neutral lipid, a cholesterol, and/or a PEG lipid. Lipid based carriers are further described below in § 5.7.1.

### 5.7.1 Lipid Based Carriers

In some embodiments, a SARS-CoV-2 spike protein or polypeptide (e.g., a SARS-CoV-2 spike protein or polypeptide immunogen (or an immunogenic fragment and/or immunogenic variant thereof)) described herein, a nucleic acid molecule comprising a coding region encoding the SARS-CoV-2 spike protein or polypeptide (e.g., a SARS-CoV-2 spike protein or polypeptide immunogen (or an immunogenic fragment and/or immunogenic variant thereof)) described herein, and/or a vector comprising a nucleic acid molecule comprising a coding region encoding the SARS-CoV-2 spike protein or polypeptide (e.g., a SARS-CoV-2 spike protein or polypeptide immunogen (or an immunogenic fragment and/or immunogenic variant thereof)) described herein is encapsulated or associated with one or more lipids (e.g., cationic lipids and/or neutral lipids), thereby forming lipid-based carriers such as LNPs, liposomes, lipoplexes, or nanoliposomes.

In some embodiments, the protein, peptide, nucleic acid molecule (e.g., RNA (e.g., mRNA), DNA), and/or vector is encapsulated in one or more lipids (e.g., cationic lipids and/or neutral lipids), thereby forming lipid-based carriers such as LNPs, liposomes, lipoplexes, or nanoliposomes. In some embodiments the protein, peptide, nucleic acid molecule (e.g., RNA (e.g., mRNA), DNA), and/or vector is associated with one or more lipids (e.g., cationic lipids and/or neutral lipids), thereby forming lipid-based carriers such as LNPs, liposomes, lipoplexes, or nanoliposomes.

In some embodiments, the protein, peptide, nucleic acid molecule (e.g., RNA (e.g., mRNA), DNA), and/or vector is encapsulated in an LNP (e.g., as described herein). In some embodiments the protein, peptide, nucleic acid molecule (e.g., RNA (e.g., mRNA), DNA), and/or vector is associated with an LNP (e.g., as described herein). LNPs are described in further detail in § 5.7.1.1. The use of LNPs for mRNA delivery is further detailed in e.g., Hou X et al. Lipid nanoparticles for mRNA delivery. Nat Rev Mater. 2021; 6(12):1078-1094. doi: 10.1038/s41578-021-00358-0. Epub 2021 Aug. 10. PMID: 34394960; PMCID: PMC8353930, the entire contents of which is incorporated herein by reference for all purposes.

The proteins, peptides, nucleic acid molecules (e.g., RNA (e.g., mRNA), DNA), and/or vectors may be completely or partially located in the interior space of the LNPs, liposomes, lipoplexes, and/or nanoliposomes, within the lipid layer/membrane, or associated with the exterior surface of the lipid layer/membrane. One purpose of incorporating proteins, peptides, nucleic acid molecules (e.g., RNA (e.g., mRNA), DNA), and/or vectors into LNPs, liposomes, lipoplexes, and/or nanoliposomes is to protect the proteins, peptides, nucleic acid molecules (e.g., RNA (e.g., mRNA), DNA), and/or vectors from an environment which may contain enzymes or chemicals or conditions that degrade the proteins, peptides, nucleic acid molecules (e.g., RNA (e.g., mRNA), DNA), and/or vectors and/or systems or receptors that cause the rapid excretion of the proteins, peptides, nucleic acid molecules (e.g., RNA (e.g., mRNA), DNA), and/or vectors. Moreover, incorporating proteins, peptides, nucleic acid molecules (e.g., RNA (e.g., mRNA), DNA), and/or vectors into LNPs, liposomes, lipoplexes, and/or nanoliposomes may promote the uptake of the proteins, peptides, nucleic acid molecules (e.g., RNA (e.g., mRNA), DNA), and/or vectors, and hence, may enhance the therapeutic effect of the proteins, peptides, nucleic acid molecules (e.g., RNA (e.g., mRNA), DNA), and/or vectors. Accordingly, incorporating a protein, peptide, nucleic acid molecule (e.g., RNA (e.g., mRNA), DNA), and/or vector (e.g., described herein), into LNPs, liposomes, lipoplexes, and/or nanoliposomes may be particularly suitable for a pharmaceutical composition described herein, e.g., for intramuscular and/or intradermal administration.

LNPs, liposomes, lipoplexes, and/or nanoliposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 nm and 500 nm in diameter. In some embodiments, the LNPs, liposomes, lipoplexes, and/or nanoliposomes has a diameter from about 10 to 500 nm, 10 to 400 nm, 10 to 300 nm, 10 to 200 nm, 10 to 100 nm, or 10 to 50 nm. In some embodiments, the LNPs, liposomes, lipoplexes, and/or nanoliposomes has a diameter of at least about 10 nm, 20 nm, 30 nm, 40 nm, 50 nm, 60 nm, 70 nm, 80 nm, 90 nm, 100 nm, 150 nm, 200 nm, 250 nm, 300 nm, 350 nm, 400 nm, 450 nm, or 500 nm.

### 5.7.1.1 Lipid Nanoparticles (LNPs)

In some embodiments, the SARS-CoV-2 spike protein or polypeptide (e.g., a SARS-CoV-2 spike protein or polypeptide immunogen (or an immunogenic fragment and/or immunogenic variant thereof)) described herein, the nucleic acid molecule comprising a coding region encoding the SARS-CoV-2 spike protein or polypeptide (e.g., a SARS-CoV-2 spike protein or polypeptide immunogen (or an immunogenic fragment and/or immunogenic variant thereof)) described herein, or a vector comprising a nucleic acid molecule comprising a coding region encoding the SARS-CoV-2 spike protein or polypeptide (e.g., a SARS-CoV-2 spike protein or polypeptide immunogen (or an immunogenic fragment and/or immunogenic variant thereof)) described herein is comprised in an LNP. In some embodiments, LNPs include one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; the entire contents of which is incorporated herein by reference for all purposes); one or more sterols (e.g., cholesterol). In embodiments, an LNP preparation comprises a cationic lipid, a neutral lipid, a cholesterol, and a PEG lipid, and has a mean particle size of between 50-200 nm, e.g., between 80 nm and 160 nm.

Lipids that can be used in nanoparticle formations (e.g., LNPs) include, for example those described in Table 4 of WO2019217941, which is incorporated herein by reference—e.g., a lipid-containing nanoparticle can include one or more of the lipids in Table 4 of WO2019217941. LNPs can include additional elements, such as polymers, such as the polymers described in Table 5 of WO2019217941, the entire contents of which is incorporated by reference herein for all purposes.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (the entire contents of which is incorporated by reference herein for all purposes), and combinations of the foregoing.

In some embodiments, sterols that can be incorporated into lipid nanoparticles include one or more of cholesterol or cholesterol derivatives, such as those in WO2009/127060 or US2010/0130588, the entire contents of which is incorporated by reference herein for all purposes. Additional exemplary sterols include phytosterols, including those described in Eygeris et al. (2020), dx.doi.org/10.1021/acs.nanolett.Oc01386, the entire contents of which is incorporated by reference herein for all purposes.

In some embodiments, the lipid particle includes an ionizable lipid, a non-cationic lipid, a conjugated lipid that inhibits aggregation of particles, and a sterol. The amounts of these components can be varied independently and to achieve desired properties. For example, in some embodiments, the lipid nanoparticle includes an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA (e.g., circular polyribonucleotide, linear polyribonucleotide)) described herein includes,

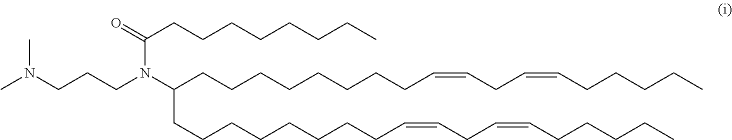 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP including Formula (i) is used to deliver a polyribonucleotide (e.g., a circular polyribonucleotide, a linear polyribonucleotide) composition described herein to cells.

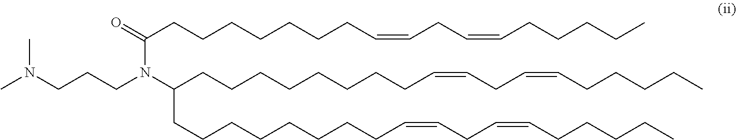 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP including Formula (ii) is used to deliver a polyribonucleotide (e.g., a circular polyribonucleotide, a linear polyribonucleotide) composition described herein to cells.

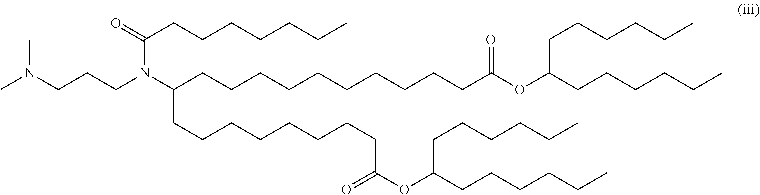 [SMILES: CCCCCCCC(=O)N(CCCN(C)C)C(CCCCCCCCCCC(=O)OC(CCCCCC)CCCCCC)CCCCCCCCC(=O)OC(CCCCCC)CCCCCC]

In some embodiments an LNP including Formula (iii) is used to deliver a polyribonucleotide (e.g., a circular polyribonucleotide, a linear polyribonucleotide) composition described herein to cells.

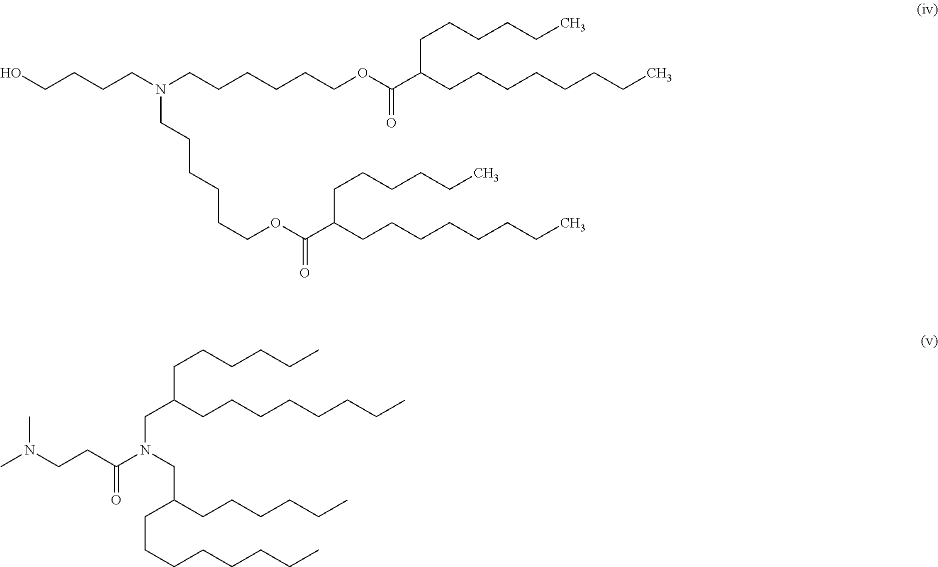

In some embodiments an LNP including Formula (v) is used to deliver a polyribonucleotide (e.g., a circular polyribonucleotide, a linear polyribonucleotide) composition described herein to cells.

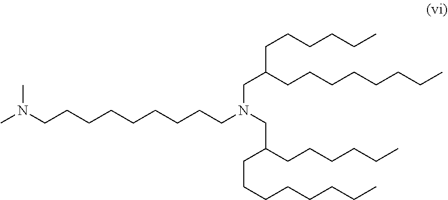 [SMILES: CCCCCCCCC(CCCCCC)CN(CCCCCCCCCN(C)C)CC(CCCCCC)CCCCCCCC]

In some embodiments an LNP including Formula (vi) is used to deliver a polyribonucleotide (e.g., a circular polyribonucleotide, a linear polyribonucleotide) composition described herein to cells.

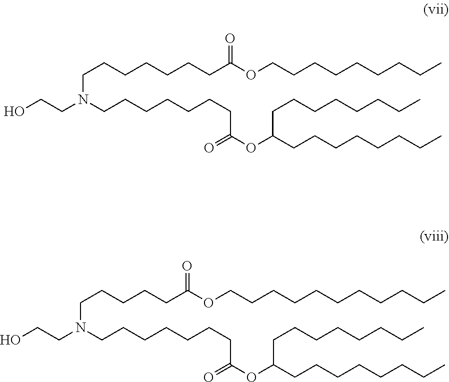 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP including Formula (viii) is used to deliver a polyribonucleotide (e.g., a circular polyribonucleotide, a linear polyribonucleotide) composition described herein to cells.

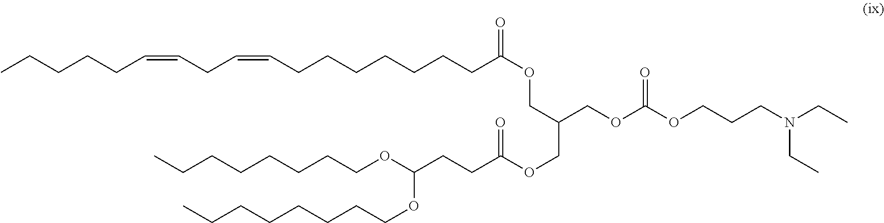 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP including Formula (ix) is used to deliver a polyribonucleotide (e.g., a circular polyribonucleotide, a linear polyribonucleotide) composition described herein to cells.

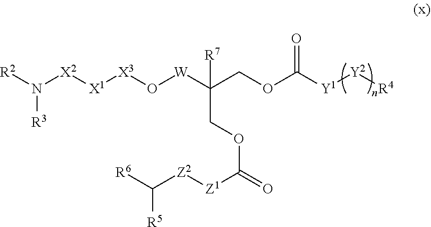

wherein
X¹ is O, NR¹, or a direct bond, X² is C2-5 alkylene, X³ is C(═O) or a direct bond, R¹ is H or Me, R³ is C1-3 alkyl, R² is C1-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

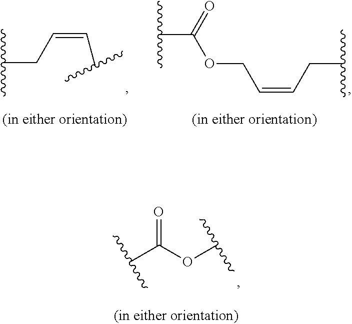 [SMILES: */C=C\C* ; *C/C=C\COC(*)=O ; *OC(*)=O]

n is 0 to 3, R⁴ is C1-15 alkyl, Z¹ is C1-6 alkylene or a direct bond,
Z² is

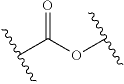 [SMILES: *OC(*)=O]

(in either orientation) or absent, provided that if Z¹ is a direct bond, Z² is absent;
R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls, X¹ is O, X² is linear C3 alkylene, X³ is C(═O), Y¹ is linear Ce alkylene, (Y²)n-R⁴ is

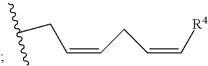

R⁴ is linear C5 alkyl, Z¹ is C2 alkylene, Z² is absent, W is methylene, and R⁷ is H, then R⁵ and R⁶ are not Cx alkoxy.

In some embodiments an LNP including Formula (xii) is used to deliver a polyribonucleotide (e.g., a circular polyribonucleotide, a linear polyribonucleotide) composition described herein to cells.

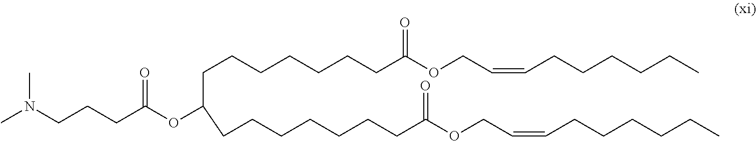 [SMILES: CCCCCC/C=C\COC(=O)CCCCCCCC(CCCCCCCC(=O)OC/C=C\CCCCCC)OC(=O)CCCN(C)C]

In some embodiments an LNP including Formula (xi) is used to deliver a polyribonucleotide (e.g., a circular polyribonucleotide, a linear polyribonucleotide) composition described herein to cells.

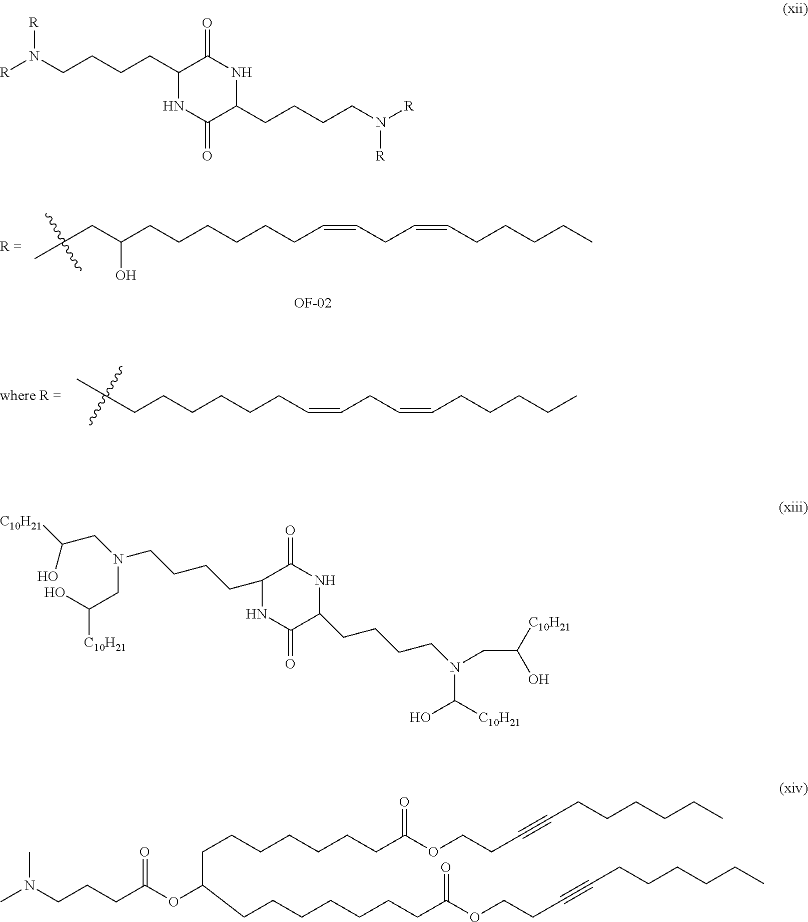

In some embodiments an LNP includes a compound of Formula (xiii) and a compound of Formula (xiv).

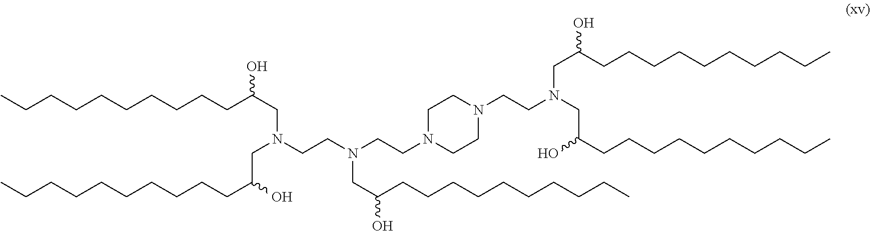 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP including Formula (xv) is used to deliver a polyribonucleotide (e.g., a circular polyribonucleotide, a linear polyribonucleotide) composition described herein to cells.

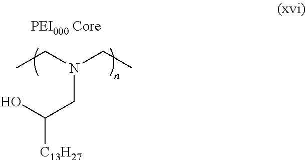

In some embodiments an LNP including a formulation of Formula (xvi) is used to deliver a polyribonucleotide (e.g., a circular polyribonucleotide, a linear polyribonucleotide) composition described herein to cells.

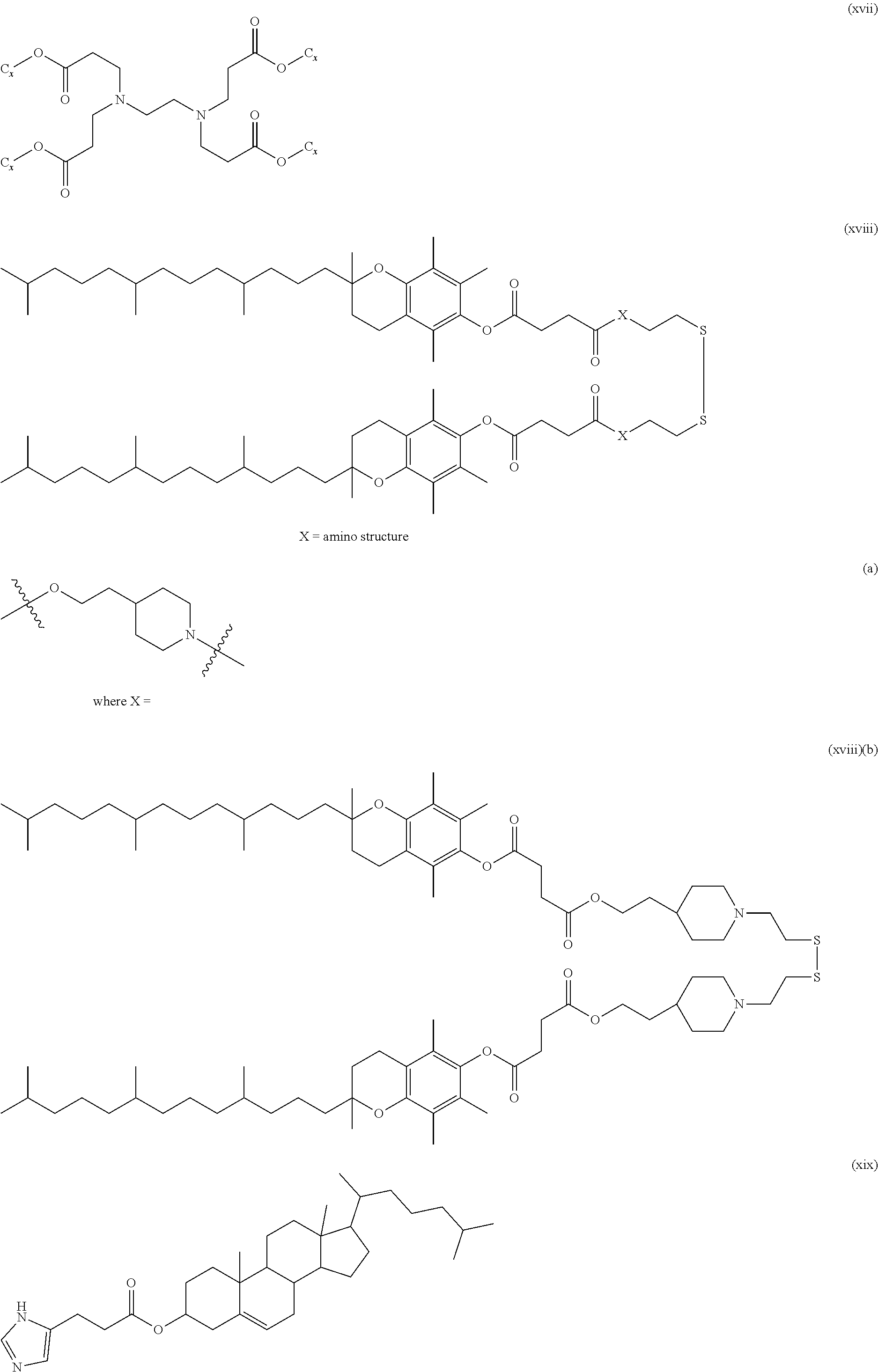

In some embodiments, a lipid compound used to form lipid nanoparticles for the delivery of compositions described herein, e.g., nucleic acid (e.g., RNA (e.g., circular polyribonucleotide, linear polyribonucleotide)) described herein is made by one of the following reactions:

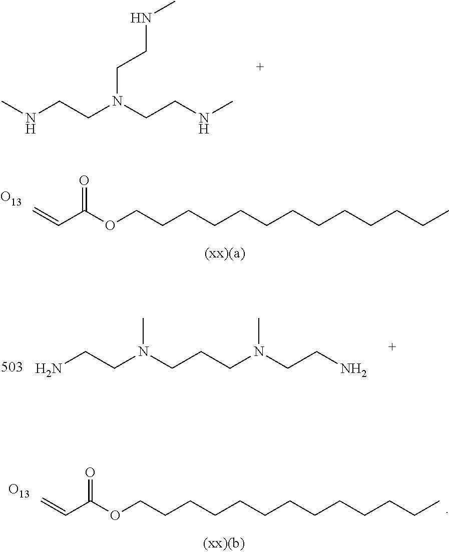

In some embodiments an LNP including Formula (xxi) is used to deliver a polyribonucleotide (e.g., a circular polyribonucleotide, a linear polyribonucleotide) composition described herein to cells. In some embodiments the LNP of Formula (xxi) is an LNP described by WO2021113777 (e.g., a lipid of Formula (1) such as a lipid of Table 1 of WO2021113777, the entire contents of which is incorporated by reference herein for all purposes).

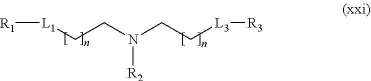

wherein
each n is independently an integer from 2-15; L₁ and L₃ are each independently —OC(O)—* or —C(O)O—*, wherein “*” indicates the attachment point to R₁ or R₃;
R₁ and R₃ are each independently a linear or branched C₉-C₂₀ alkyl or C₉-C₂₀ alkenyl, optionally substituted by one or more substituents selected from a group consisting of oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkyl sulfonyl, and alkyl sulfonealkyl; and
R₂ is selected from a group consisting of:

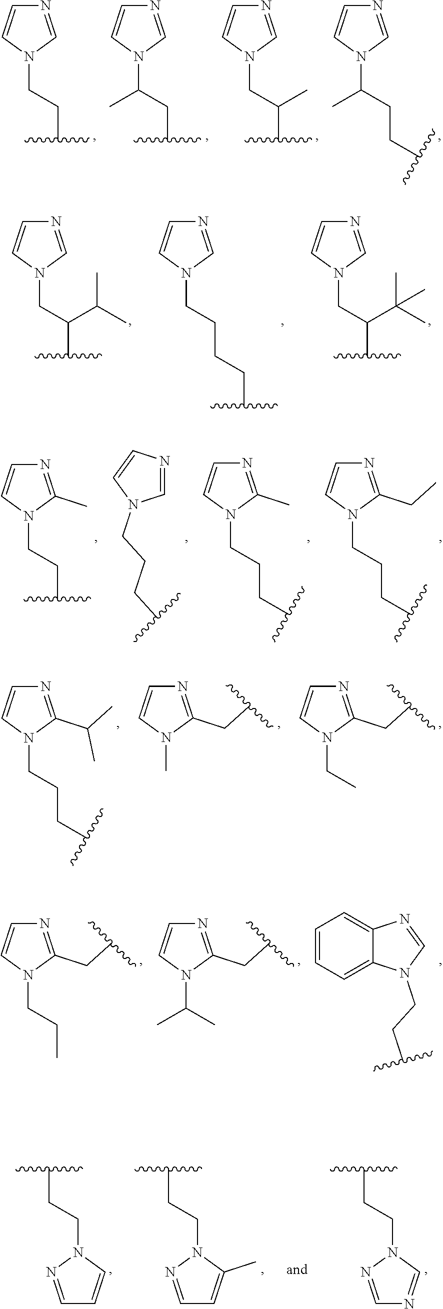

In some embodiments an LNP including Formula (xxii) is used to deliver a polyribonucleotide (e.g., a circular polyribonucleotide, a linear polyribonucleotide) composition described herein to cells. In some embodiments the LNP of Formula (xxii) is an LNP described by WO2021113777 (e.g., a lipid of Formula (2) such as a lipid of Table 2 of WO2021113777).

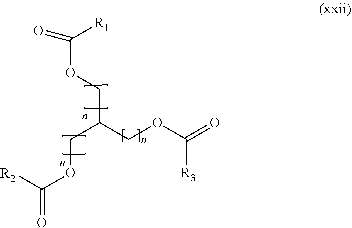

wherein
each n is independently an integer from 1-15;
R₁ and R₂ are each independently selected from a group consisting of:

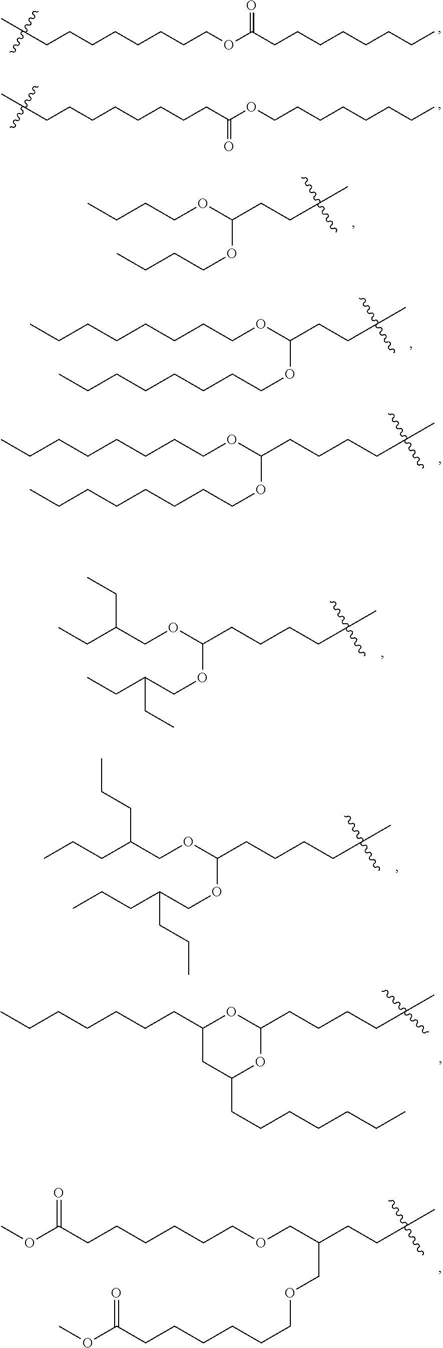

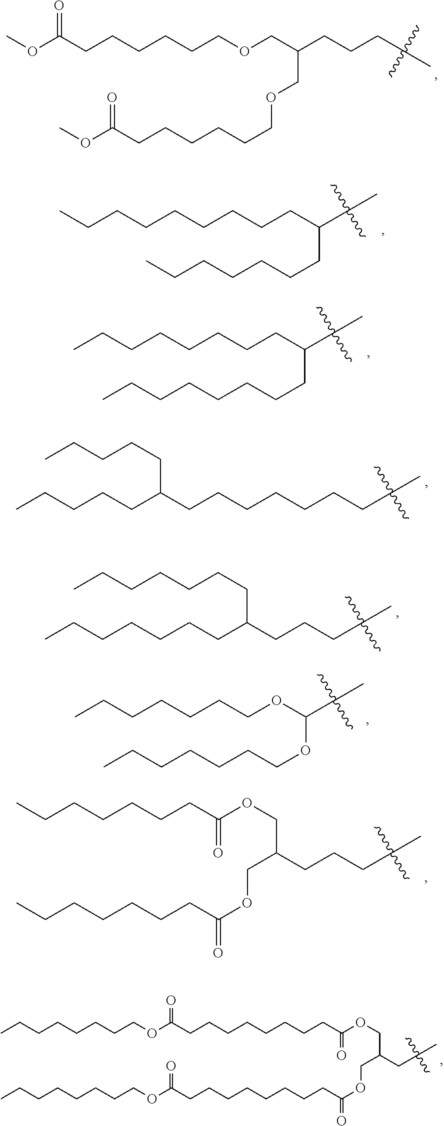

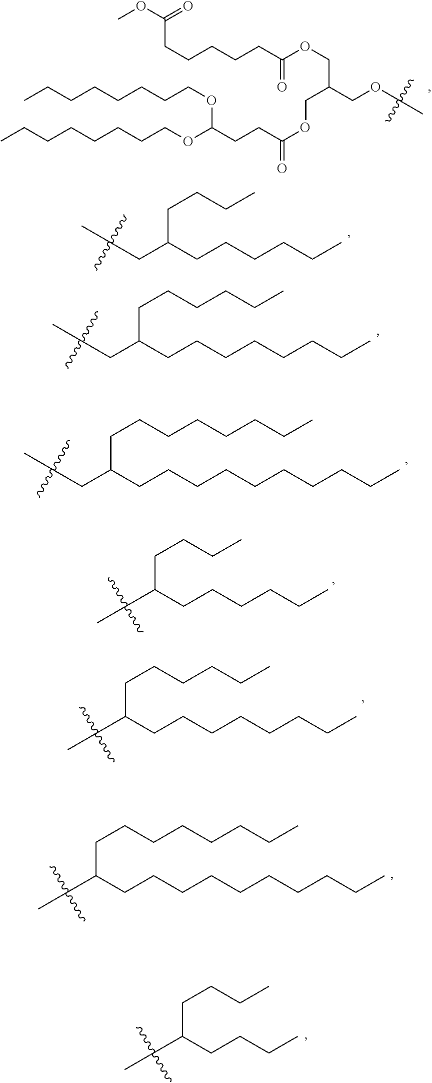

R₃ is selected from a group consisting of:

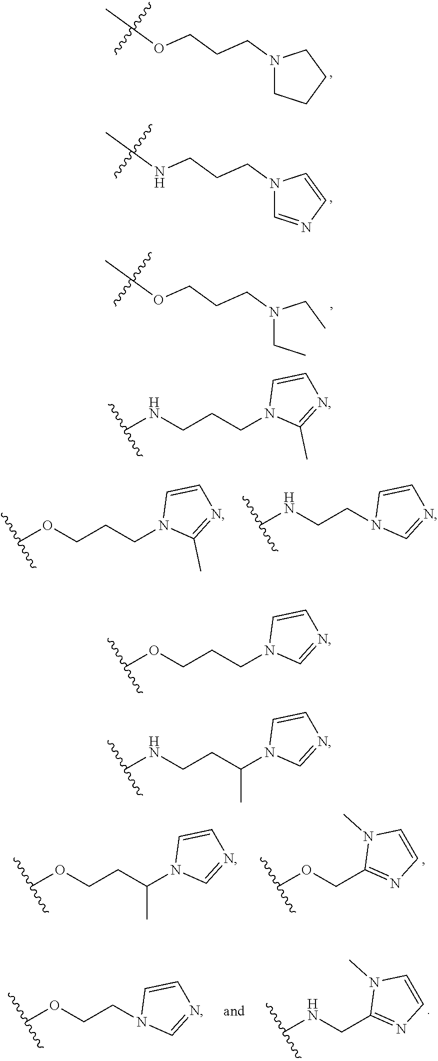

In some embodiments an LNP including Formula (xxiii) is used to deliver a polyribonucleotide (e.g., a circular polyribonucleotide, a linear polyribonucleotide) composition described herein to cells. In some embodiments the LNP of Formula (xxiii) is an LNP described by WO2021113777 (e.g., a lipid of Formula (3) such as a lipid of Table 3 of WO2021113777).

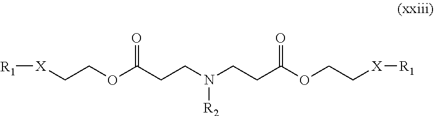

wherein
X is selected from —O—, —S—, or —OC(O)—*, wherein * indicates the attachment point to R₁;
R₁ is selected from a group consisting of:

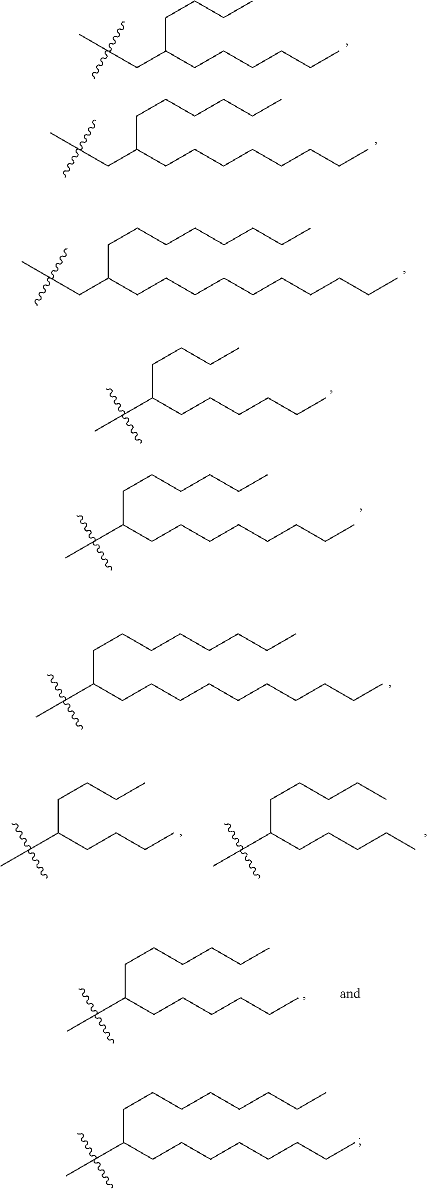

and R₂ is selected from a group consisting of:

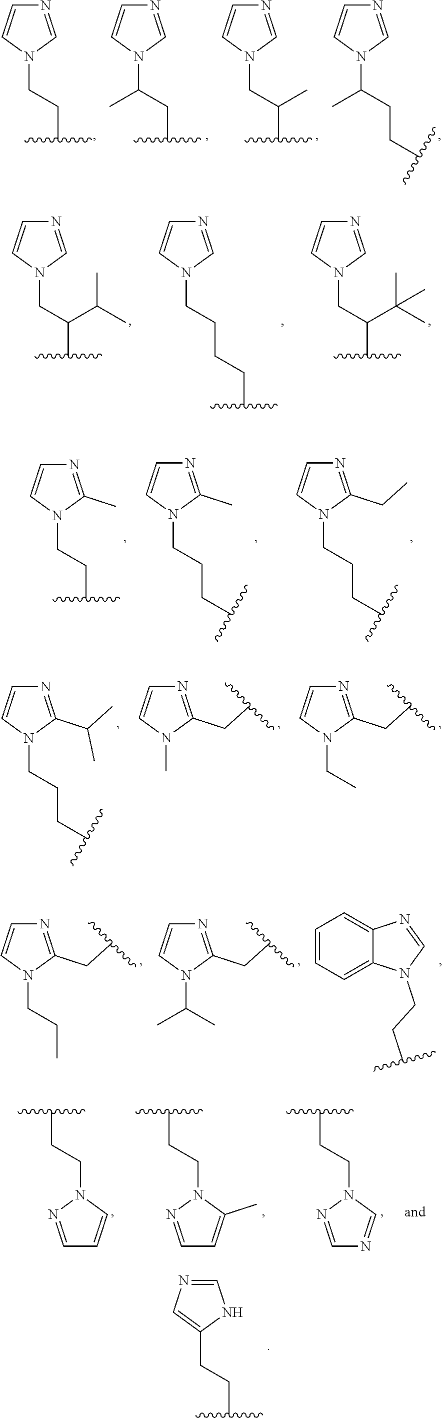

In some embodiments, a composition described herein (e.g., a nucleic acid (e.g., a circular polyribonucleotide, a linear polyribonucleotide) or a protein) is provided in an LNP that includes an ionizable lipid. In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LP01), e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy)heptadecanedioate (L319), e.g., as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, e.g., Structure (I) from WO2020/106946 (the entire contents of which is incorporated by reference herein for all purposes).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle includes a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol, and polymer conjugated lipids. In some embodiments, the cationic lipid may be an ionizable cationic lipid. An exemplary cationic lipid as disclosed herein may have an effective pKa over 6.0. In embodiments, a lipid nanoparticle may include a second cationic lipid having a different effective pKa (e.g., greater than the first effective pKa), than the first cationic lipid. A lipid nanoparticle may include between 40 and 60 mol percent of a cationic lipid, a neutral lipid, a steroid, a polymer conjugated lipid, and a therapeutic agent, e.g., a nucleic acid (e.g., RNA (e.g., a circular polyribonucleotide, a linear polyribonucleotide)) described herein, encapsulated within or associated with the lipid nanoparticle. In some embodiments, the nucleic acid is co-formulated with the cationic lipid. The nucleic acid may be adsorbed to the surface of an LNP, e.g., an LNP including a cationic lipid. In some embodiments, the nucleic acid may be encapsulated in an LNP, e.g., an LNP including a cationic lipid. In some embodiments, the lipid nanoparticle may include a targeting moiety, e.g., coated with a targeting agent. In embodiments, the LNP formulation is biodegradable. In some embodiments, a lipid nanoparticle including one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule.

Exemplary ionizable lipids that can be used in lipid nanoparticle formulations include, without limitation, those listed in Table 1 of WO2019051289, the entire contents of which is incorporated by reference herein for all purposes. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; III-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-O13 or 503-O13 of Whitehead et al; TS-P4C2 of U.S. Pat. No. 9,708,628; I of WO2020/106946; I of WO2020/106946; and (1), (2), (3), or (4) of WO2021/113777. Exemplary lipids further include a lipid of any one of Tables 1-16 of WO2021/113777. The entire contents of each reference is incorporated by reference herein for all purposes

In some embodiments, the ionizable lipid is MC3 (6Z,9Z,28Z,3 lZ)-heptatriaconta-6,9,28,3 1-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (the entire contents of which is incorporated by reference herein for all purposes). In some embodiments, the ionizable lipid is (13Z,16Z)-A,A-dimethyl-3-nonyldocosa-13, 16-dien-1-amine (Compound 32), e.g., as described in Example 11 of WO2019051289A9 (the entire contents of which is incorporated by reference herein for all purposes). In some embodiments, the ionizable lipid is Compound 6 or Compound 22, e.g., as described in Example 12 of WO2019051289A9 (the entire contents of which is incorporated by reference herein for all purposes).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.Oc01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the entire contents of which is incorporated by reference herein for all purposes.

In some embodiments, the non-cationic lipid is oleic acid or a compound of Formula I, II, or IV of US2018/0028664, the entire contents of which is incorporated by reference herein for all purposes. The non-cationic lipid can include, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not include any phospholipids.

In some aspects, the lipid nanoparticle can further include a component, such as a sterol, to provide membrane integrity. One exemplary sterol that can be used in the lipid nanoparticle is cholesterol and derivatives thereof. Non-limiting examples of cholesterol derivatives include polar analogues such as 5a-cholestanol, 53-coprostanol, cholesteryl-(2-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in PCT publication WO2009/127060 and US patent publication US2010/0130588, the entire contents of each of which is incorporated by reference herein for all purposes.

In some embodiments, the component providing membrane integrity, such as a sterol, can include 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can include a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the entire contents of each of which is incorporated by reference herein for all purposes. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the entire contents of each of which is incorporated by reference herein for all purposes. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl] carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid includes PEG-DMG, 1,2-dimyristoyl-sn-glycero-3-phosphoethanolamine-N-[methoxy(polyethylene glycol)-2000]. In some embodiments, the PEG-lipid includes a structure selected from:

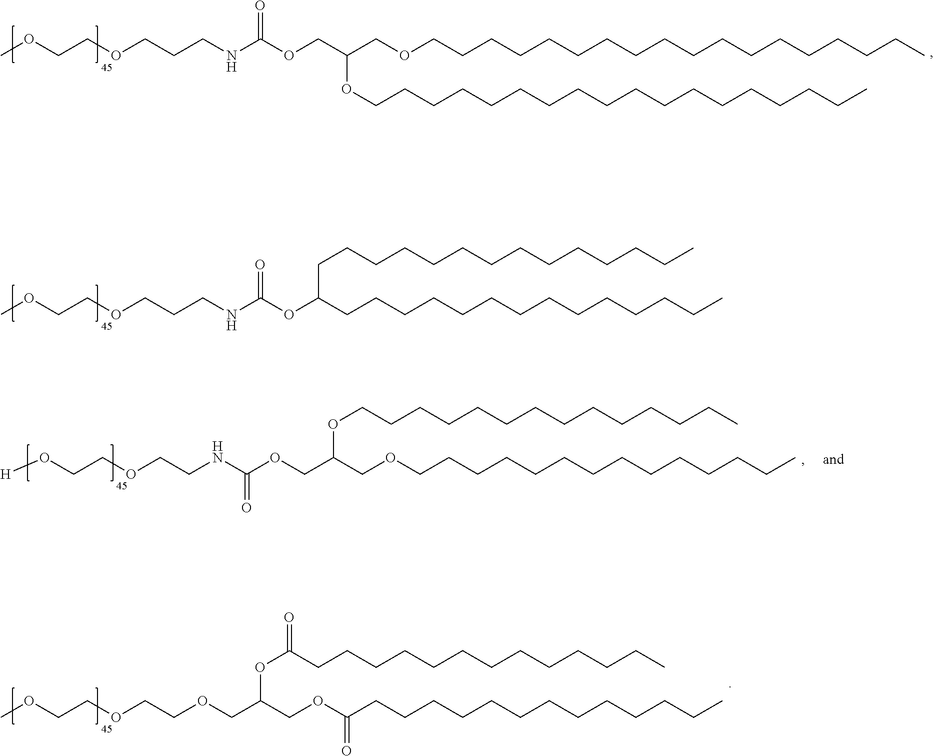

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the entire contents of which is incorporated by reference herein for all purposes.

In some embodiments, the PEG or the conjugated lipid can include 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can include 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition includes 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example including 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation includes ionizable lipid, phospholipid, cholesterol and a PEG-ylated lipid in a molar ratio of 50:10:38.5:1.5. In some other embodiments, the lipid particle formulation includes ionizable lipid, cholesterol and a PEG-ylated lipid in a molar ratio of 60:38.5:1.5.

In some embodiments, the lipid particle includes ionizable lipid, non-cationic lipid (e.g., phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle includes ionizable lipid/non-cationic-lipid/sterol/conjugated lipid at a molar ratio of 50:10:38.5:1.5.

In an aspect, the disclosure provides a lipid nanoparticle formulation including phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

In some embodiments, the LNPs include biodegradable, ionizable lipids. In some embodiments, the LNPs include (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of WO2019/067992, WO/2017/173054, WO2015/095340, and WO2014/136086, the entire contents of each of which is incorporated by reference herein for all purposes, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally include one or more coatings. In some embodiments, a LNP may be formulated in a capsule, film, or table having a coating. A capsule, film, or tablet including a composition described herein may have any useful size, tensile strength, hardness, or density.

Additional exemplary lipids, formulations, methods, and characterization of LNPs are taught by WO2020/061457 and WO2021/113777, the entire contents of each of which is incorporated by reference herein for all purposes. Further exemplary lipids, formulations, methods, and characterization of LNPs are taught by Hou et al. Lipid nanoparticles for mRNA delivery. Nat Rev Mater (2021). doi.org/10.1038/s41578-021-00358-0, which is incorporated herein by reference in its entirety (see, for example, exemplary lipids and lipid derivatives of FIG. 2 of Hou et al.), the entire contents of which is incorporated by reference herein for all purposes.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), the entire contents of which is incorporated by reference herein for all purposes.

LNP formulations optimized for the delivery of CRISPR-Cas systems, e.g., Cas9-gRNA RNP, gRNA, Cas9 mRNA, are described in WO2019067992 and WO2019067910, the entire contents of each of which is incorporated by reference herein for all purposes, and are useful for delivery of circular polyribonucleotides and linear polyribonucleotides described herein.

Additional specific LNP formulations useful for delivery of nucleic acids (e.g., circular polyribonucleotides, linear polyribonucleotides) are described in U.S. Pat. Nos. 8,158,601 and 8,168,775, the entire contents of each of which is incorporated by reference herein for all purposes, which include formulations used in patisiran, sold under the name ONPATTRO.

Exemplary dosing of polyribonucleotide (e.g., a circular polyribonucleotide, a linear polyribonucleotide) LNP may include about 0.1, 0.25, 0.3, 0.5, 1, 2, 3, 4, 5, 6, 8, 10, or 100 mg/kg (RNA). Exemplary dosing of AAV including a polyribonucleotide (e.g., a circular polyribonucleotide, a linear polyribonucleotide) may include an MOI of about 10¹¹, 10¹², 10¹³, and 10¹⁴ vg/kg.

### 5.8 Pharmaceutical Compositions

In one aspect, provided herein are pharmaceutical compositions comprising a SARS-CoV-2 spike protein or polypeptide (e.g., a SARS-CoV-2 spike protein or polypeptide immunogen (or an immunogenic fragment and/or immunogenic variant thereof)) described herein (or a fusion or conjugate thereof), a nucleic acid molecule described herein (e.g., a nucleic acid molecule comprising a coding region encoding a SARS-CoV-2 spike protein or polypeptide (e.g., a SARS-CoV-2 spike protein or polypeptide immunogen (or an immunogenic fragment and/or immunogenic variant thereof)) (or a fusion or conjugate thereof), a vector described herein (e.g., a vector comprising a nucleic acid molecule described herein (e.g., a nucleic acid molecule comprising a coding region encoding a SARS-CoV-2 spike protein or polypeptide (e.g., a SARS-CoV-2 spike protein or polypeptide immunogen (or an immunogenic fragment and/or immunogenic variant thereof)), a carrier described herein (e.g., a carrier comprising a SARS-CoV-2 spike protein or polypeptide (e.g., a SARS-CoV-2 spike protein or polypeptide immunogen (or an immunogenic fragment and/or immunogenic variant thereof)) described herein or a nucleic acid molecule described herein (e.g., a nucleic acid molecule comprising a coding region encoding a SARS-CoV-2 spike protein (e.g., a SARS-CoV-2 spike protein or polypeptide immunogen (or an immunogenic fragment and/or immunogenic variant thereof))), and/or a composition described herein (e.g., a vaccine composition), and a pharmaceutically acceptable excipient (see, e.g., Remington's Pharmaceutical Sciences (1990) Mack Publishing Co., Easton, PA, the entire contents of which is incorporated by reference herein for all purposes).

In one aspect, also provided herein are methods of making pharmaceutical compositions described herein comprising providing a SARS-CoV-2 spike protein or polypeptide (e.g., a SARS-CoV-2 spike protein or polypeptide immunogen (or an immunogenic fragment and/or immunogenic variant thereof)) described herein, a nucleic acid molecule described herein (e.g., a nucleic acid molecule comprising a coding region encoding a SARS-CoV-2 spike protein (e.g., a SARS-CoV-2 spike protein or polypeptide immunogen (or an immunogenic fragment and/or immunogenic variant thereof)) described herein), a vector described herein (e.g., a vector comprising a nucleic acid molecule described herein (e.g., a nucleic acid molecule comprising a coding region encoding a SARS-CoV-2 spike protein or polypeptide (e.g., a SARS-CoV-2 spike protein or polypeptide immunogen (or an immunogenic fragment and/or immunogenic variant thereof)))), a carrier described herein (e.g., a carrier comprising a SARS-CoV-2 spike protein (or polypeptide e.g., immunogen (or an immunogenic fragment and/or immunogenic variant thereof)) described herein or a nucleic acid molecule described herein (e.g., a nucleic acid molecule encoding a SARS-CoV-2 spike protein or polypeptide (e.g., a SARS-CoV-2 spike protein or polypeptide immunogen (or an immunogenic fragment and/or immunogenic variant thereof))), and/or a composition described herein (e.g., a vaccine composition), and formulating it into a pharmaceutically acceptable composition by the addition of one or more pharmaceutically acceptable excipient.

Acceptable excipients (e.g., carriers and stabilizers) are preferably nontoxic to recipients at the dosages and concentrations employed, and include buffers such as phosphate, citrate, or other organic acids; antioxidants including ascorbic acid or methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; or m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, or other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

A pharmaceutical composition may be formulated for any route of administration to a subject. The skilled person knows the various possibilities to administer a pharmaceutical composition described herein a in order to induce an immune response to the immunogens(s) and/or antigen(s) in the pharmaceutical composition. Non-limiting embodiments include parenteral administration, such as intramuscular, intradermal, subcutaneous, transcutaneous, or mucosal administration, e.g., inhalation, intranasal, oral, and the like. In one embodiment, the pharmaceutical composition is formulated for administration by intramuscular, intradermal, or subcutaneous injection. In one embodiment, the pharmaceutical composition is formulated for administration by intramuscular injection. In one embodiment, the pharmaceutical composition is formulated for administration by intradermal injection. In one embodiment, the pharmaceutical composition is formulated for administration by subcutaneous injection. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, or other such agents, such as for example, sodium acetate, sorbitan monolaurate, triethanolamine oleate or cyclodextrins. In some embodiments, the pharmaceutical composition is formulated in a single dose. In some embodiments, the pharmaceutical compositions if formulated as a multi-dose.

Pharmaceutically acceptable excipients used in the parenteral preparations described herein include for example, aqueous vehicles, nonaqueous vehicles, antimicrobial agents, isotonic agents, buffers, antioxidants, local anesthetics, suspending and dispersing agents, emulsifying agents, sequestering or chelating agents or other pharmaceutically acceptable substances. Examples of aqueous vehicles, which can be incorporated in one or more of the formulations described herein, include sodium chloride injection, Ringer's injection, isotonic dextrose injection, sterile water injection, dextrose or lactated Ringer's injection. Nonaqueous parenteral vehicles, which can be incorporated in one or more of the formulations described herein, include fixed oils of vegetable origin, cottonseed oil, corn oil, sesame oil or peanut oil. Antimicrobial agents in bacteriostatic or fungistatic concentrations can be added to the parenteral preparations described herein and packaged in multiple-dose containers, which include phenols or cresols, mercurials, benzyl alcohol, chlorobutanol, methyl and propyl p-hydroxybenzoic acid esters, thimerosal, benzalkonium chloride or benzethonium chloride. Isotonic agents, which can be incorporated in one or more of the formulations described herein, include sodium chloride or dextrose. Buffers, which can be incorporated in one or more of the formulations described herein, include phosphate or citrate. Antioxidants, which can be incorporated in one or more of the formulations described herein, include sodium bisulfate. Local anesthetics, which can be incorporated in one or more of the formulations described herein, include procaine hydrochloride. Suspending and dispersing agents, which can be incorporated in one or more of the formulations described herein, include sodium carboxymethylcelluose, hydroxypropyl methylcellulose or polyvinylpyrrolidone. Emulsifying agents, which can be incorporated in one or more of the formulations described herein, include Polysorbate 80 (TWEEN® 80). A sequestering or chelating agent of metal ions, which can be incorporated in one or more of the formulations described herein, is EDTA. Pharmaceutical carriers, which can be incorporated in one or more of the formulations described herein, also include ethyl alcohol, polyethylene glycol or propylene glycol for water miscible vehicles; or sodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether therapy is prophylactic or therapeutic. Therapeutic dosages are preferably titrated to optimize safety and efficacy.

### 5.9 Adjuvants

Any of the foregoing, e.g., SARS-CoV-2 spike proteins or polypeptides (e.g., immunogens (or immunogenic fragments and/or immunogenic variants thereof)) described herein (or a fusion or conjugate thereof), nucleic acid molecules described herein (e.g., nucleic acid molecules comprising a coding region encoding a SARS-CoV-2 spike protein or polypeptide (e.g., a SARS-CoV-2 spike protein or polypeptide immunogen (or an immunogenic fragment and/or immunogenic variant thereof)) (or a fusion or conjugate thereof), vectors described herein (e.g., vectors comprising a nucleic acid molecule described herein (e.g., a nucleic acid molecule comprising a coding region encoding a SARS-CoV-2 spike protein or polypeptide (e.g., a SARS-CoV-2 spike protein or polypeptide immunogen (or an immunogenic fragment and/or immunogenic variant thereof)), carriers described herein (e.g., a carrier comprising a SARS-CoV-2 spike protein or polypeptide (e.g., a SARS-CoV-2 spike protein or polypeptide immunogen (or an immunogenic fragment and/or immunogenic variant thereof)) described herein or a nucleic acid molecule described herein (e.g., a nucleic acid molecule comprising a coding region encoding a SARS-CoV-2 spike protein or polypeptide (e.g., a SARS-CoV-2 spike protein or polypeptide immunogen (or an immunogenic fragment and/or immunogenic variant thereof))), compositions described herein (e.g., a vaccine composition), and/or pharmaceutical compositions described herein may be co-formulated with and/or administered in combination with an adjuvant.

Adjuvants are known in the art to further increase the immune response (e.g., to an immunogen). General categories of adjuvants include, but are not limited to, inorganic adjuvants, small molecule adjuvants, oil in water emulsions, lipids, polymers, peptides, peptidoglycans, carbohydrates, polysaccharides, RNA-based adjuvants, DNA-based adjuvants, viral particles, bacterial adjuvants, nanoparticles (e.g., inorganic nanoparticles), and multi-component adjuvants. Examples of adjuvants include, but are not limited to, aluminum salts such as aluminum hydroxide and/or aluminum phosphate; oil-emulsion compositions (or oil-in-water compositions), including squalene-water emulsions, such as MF59 (see, e.g., WO90/14837, the entire contents of which is incorporated herein by reference for all purposes), MF59, AS03, and Montanide; saponin formulations, such as for example QS21 and Immunostimulating Complexes (ISCOMS) (see, e.g., U.S. Pat. No. 5,057,540; WO90/03184, WO96/11711, WO2004/004762, WO2005/002620, the entire contents of each of which is incorporated herein by reference for all purposes); protamine or a protamine salt (e.g., protamine sulfate); calcium salt; bacterial or microbial derivatives, examples of which include monophosphoryl lipid A (MPL), 3-O-deacylated MPL (3dMPL), CpG-motif containing oligonucleotides, ADP-ribosylating bacterial toxins or mutants thereof, such as E. coli heat labile enterotoxin LT, cholera toxin CT, and the like; eukaryotic proteins (e.g., antibodies or fragments thereof (e.g., directed against the antigen itself or CD1a, CD3, CD7, CD80) and ligands to receptors (e.g., CD40L, GMCSF, GCSF, etc.).

Exemplary RNA-based adjuvants include, but are not limited to, Poly IC, Poly IC:LC, hairpin RNAs, e.g., with a 5′PPP containing sequence, viral sequences, polyU containing sequences, dsRNA, natural or synthetic immunostimulatory RNA sequences, nucleic acids analogs, optionally cyclic GMP-AMP or a cyclic dinucleotide such as cyclic di-GMP, and immunostimulatory base analogs, e.g., C8-substitued or an N7,C8-disubstituted guanine ribonucleotide. Exemplary DNA-based adjuvants, include, but are not limited to, CpGs, dsDNA, or natural or synthetic immunostimulatory DNA sequences. Exemplary bacteria-based adjuvants include, but are not limited, to bacterial adjuvant is flagellin, LPS, or a bacterial toxin, e.g., enterotoxins, heat-labile toxins, and Cholera toxins. Exemplary carbohydrate or polysaccharide adjuvants include, but are not limited to, dextran (branched microbial polysaccharide), dextran-sulfate, Lentinan, zymosan, Betaglucan, Deltin, Mannan, and Chitin. Exemplary small molecule adjuvants, include, but are not limited to, imiquimod, resiquimod, and gardiquimod. Exemplary lipid or polymer adjuvants, include, but are not limited to, polymeric nanoparticles (e.g., PLGA, PLG, PLA, PGA, or PHB), liposomes (e.g., Virosomes and CAF01), LNPs or a component thereof, lipopolysaccharide (LPS) (e.g., monophosphoryl lipid A (MPLA) or glucopyranosyl Lipid A (GLA)), lipopeptides (e.g., Pam2 (Pam2CSK4) or Pam3 (Pam3CSK4)), and glycolipid (e.g., trehalose dimycolate). Exemplary peptides or peptidoglycan include, but are not limited to, N-acetyl-muramyl-L-alanyl-D-isoglutamine (MDP), flagellin-fusion protein, mannose-binding lectin (MBL), cytokines, and chemokine. Exemplary inorganic nanoparticle adjuvants, include, but are not limited to, gold nanorods, silica-based nanoparticles (e.g., mesoporous silica nanoparticles (MSN)). Exemplary multicomponent adjuvants include, but are not limited to, AS01, AS03, AS04, Complete Freunds Adjuvant, and CAF01.

### 5.10 Methods of Use

Provided herein are various methods of utilizing the SARS-CoV-2 spike proteins and polypeptides (e.g., immunogens (and immunogenic fragments and/or immunogenic variants thereof)) described herein (or fusions or conjugates thereof), the nucleic acid molecules described herein (e.g., nucleic acid molecules comprising a coding region encoding a SARS-CoV-2 spike protein or polypeptide (e.g., a SARS-CoV-2 spike protein or polypeptide immunogen (or immunogenic fragment and/or immunogenic variant thereof))) (or fusions or conjugates thereof), the vectors described herein (e.g., vectors comprising a nucleic acid molecule described herein (e.g., a nucleic acid molecule comprising a coding region encoding a SARS-CoV-2 spike protein e.g., immunogen (or immunogenic fragment and/or immunogenic variant thereof)), the carriers described herein (e.g., carriers comprising a SARS-CoV-2 spike protein or polypeptide (e.g., a SARS-CoV-2 spike protein or polypeptide immunogen (or immunogenic fragment and/or immunogenic variant thereof) or a nucleic acid molecule described herein (e.g., a nucleic acid molecule comprising a encoding a SARS-CoV-2 spike protein or polypeptide (e.g., a SARS-CoV-2 spike protein or polypeptide immunogen (or immunogenic fragment and/or immunogenic variant thereof), vaccine compositions (e.g., a vaccine composition comprising any of the foregoing), and/or pharmaceutical compositions described herein (e.g., a pharmaceutical composition comprising any of the foregoing).

In some of the following aspects, the methods include administering one or more of the foregoing (e.g., protein (or a fusion or conjugate thereof), polypeptide (or a fusion or conjugate thereof), immunogen (or a fusion or conjugate thereof), nucleic acid molecule (or a fusion or conjugate thereof), vector, carrier, vaccine composition, pharmaceutical composition) to a subject. Exemplary subjects include mammals, e.g., humans, non-human mammals, e.g., non-human primates. In some embodiments, the subject is a human.

In some embodiments, the subject is, elderly, pregnant, a newborn, immunocompromised, or immunosuppressed. In some embodiments, the subject has one or more of the following cancer, heart disease, obesity, diabetes, asthma, chronic lung disease, and/or sickle cell disease. In some embodiments, the subject has a weakened immune system or weakened immune response (e.g., a weakened immune response to a vaccine). In some embodiments, the subject is immunocompromised or immunosuppressed. In some embodiments, the subject is clinically vulnerable to the infection. In some embodiments, the subject has cancer, has an autoimmune disease, has an immunodeficiency, received a bone marrow or organ transplant, is undergoing a therapy that depletes immune cells, is undergoing chemotherapy, has a chronic viral infection, post viral syndrome or post viral fatigue syndrome (e.g., HIV infection or AIDS; long Covid or persistent post-Covid syndrome), is using or has had prolonged use of an immunosuppressive medication, is currently a smoker or has a history of smoking, or is at least 50 (e.g., at least 55, 60, 65, 70, 75, 80, 85, 90, or 100) years of age. In some embodiments, the subject at least 50, 60, 65, 70, or 75 years of age. In some embodiments, the subject is at least 50, 55, 60, 65, 70, 75, 80, 85, 90, 100, 110, or 120 years of age. In some embodiments, the subject is from about 50-120, 50-110, 50-100, 50-90, 50-80, 50-70, 50-60, 60-120, 60-110, 60-100, 60-90, 60-80, 60-70, 70-120, 70-110, 70-100, 70-90, 70-80, 80-120, 80-110, 80-100, 80-90, 90-120, 90-110, or 90-100 years of age.

The dosage of one or more of the foregoing (e.g., protein, polypeptide, immunogen, nucleic acid molecule, vector, carrier, vaccine composition, pharmaceutical composition) to be administered to a subject can be determined in accordance with standard techniques well known to those of ordinary skill in the art, including the type (if any) adjuvant is used, the route of administration, and the age and weight of the subject. In some embodiments, a single dose of any one of the foregoing is administered to a subject in need thereof. In some embodiments, a series of doses of any one of the foregoing are administered to a subject in need thereof (e.g., two doses given at a set interval (e.g., 2 weeks, 3 weeks) apart or within a range (e.g., 2-6 weeks apart)). In some embodiments, the SARS-CoV-2 spike protein or polypeptide (e.g., a SARS-CoV-2 spike protein or polypeptide immunogen (or immunogenic fragment and/or immunogenic variant thereof)) described herein, the nucleic acid molecule described herein, the vector described herein, the carrier described herein, the vaccine composition, or the pharmaceutical composition described herein (e.g., any one of the foregoing) is administered in a therapeutically effective amount. In some embodiments, a dose of an mRNA molecule encoding a SARS-CoV-2 spike protein or polypeptide (e.g., a SARS-CoV-2 spike protein or polypeptide immunogen (or immunogenic fragment and/or immunogenic variant thereof)) described herein (e.g., a vaccine or pharmaceutical composition comprising the same) is between 30-200 mcg, e.g., 30 mcg, 50 mcg, 75 mcg, 100 mcg, 150 mcg, or 200 mcg.

### 5.10.1 Methods of Delivery

In one aspect, provided herein are methods of delivering (a) a SARS-CoV-2 spike protein or polypeptide described herein (e.g., a SARS-CoV-2 spike protein or polypeptide immunogen (or immunogenic fragment and/or immunogenic variant thereof) described herein) (or a fusion or conjugate thereof), (b) a nucleic acid molecule described herein (e.g., a nucleic acid molecule (e.g., an RNA molecule, e.g., an mRNA molecule) comprising a coding region encoding a SARS-CoV-2 spike protein or polypeptide described herein (e.g., a SARS-CoV-2 spike protein or polypeptide immunogen (or immunogenic fragment and/or immunogenic variant thereof) described herein) (or a fusion or conjugate thereof), (c) a vector described herein (e.g., a vector comprising a nucleic acid molecule described herein (e.g., a nucleic acid molecule (e.g., an RNA molecule, e.g., an mRNA molecule) comprising a coding region encoding a SARS-CoV-2 spike protein or polypeptide described herein (e.g., a SARS-CoV-2 spike protein or polypeptide immunogen (or immunogenic fragment and/or immunogenic variant thereof) described herein), (d) a carrier described herein (e.g., a carrier comprising a SARS-CoV-2 spike protein or polypeptide described herein (e.g., a SARS-CoV-2 spike protein or polypeptide immunogen (or immunogenic fragment and/or immunogenic variant thereof) described herein) or a nucleic acid molecule (e.g., an RNA molecule, e.g., an mRNA molecule) described herein (e.g., a nucleic acid molecule (e.g., an RNA molecule) comprising a coding region encoding a SARS-CoV-2 spike protein or polypeptide described herein (e.g., a SARS-CoV-2 spike protein or polypeptide immunogen (or immunogenic fragment and/or immunogenic variant thereof) described herein), (e) a vaccine composition described herein (e.g., a vaccine composition comprising any of the foregoing), or (f) a pharmaceutical composition described herein (e.g., a pharmaceutical composition comprising any of the foregoing) to a subject in need thereof, the method comprising administering to the subject (a) the SARS-CoV-2 spike protein or polypeptide (e.g., the SARS-CoV-2 spike protein or polypeptide immunogen (or immunogenic fragment and/or immunogenic variant thereof)), (b) the nucleic acid molecule (e.g., the RNA molecule, e.g., the mRNA molecule), (c) the vector, (d) the carrier, (e) the vaccine composition, or (f) the pharmaceutical composition to the subject, to thereby deliver the SARS-CoV-2 spike protein or polypeptide (e.g., the SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition to the subject.

In some embodiments, the SARS-CoV-2 spike protein or polypeptide (e.g., the SARS-CoV-2 spike protein or polypeptide immunogen (or immunogenic fragment and/or immunogenic variant thereof)), the nucleic acid molecule, the vector, the carrier, the vaccine composition, or the pharmaceutical composition is administered to the subject in an amount and for a time sufficient to deliver the SARS-CoV-2 spike protein or polypeptide (e.g., the SARS-CoV-2 spike protein or polypeptide immunogen (or immunogenic fragment and/or immunogenic variant thereof)), the nucleic acid molecule (e.g., the RNA molecule, e.g., an mRNA molecule), the vector, the carrier, the vaccine composition, or the pharmaceutical composition to the subject.

### 5.10.2 Methods of Inducing or Enhancing an Immune Response

In one aspect, provided herein are methods of inducing and/or enhancing an immune response in a subject in need thereof, the method comprising administering to the subject (a) a SARS-CoV-2 spike protein or polypeptide described herein (e.g., a SARS-CoV-2 spike protein or polypeptide immunogen (or immunogenic fragment and/or immunogenic variant thereof) described herein) (or a fusion or conjugate thereof), (b) a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein) (or a fusion or conjugate thereof), (c) a vector described herein (e.g., a vector comprising a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein), (d) a carrier described herein (e.g., a carrier comprising a SARS-CoV-2 spike protein or polypeptide described herein (e.g., a SARS-CoV-2 spike protein or polypeptide immunogen (or immunogenic fragment and/or immunogenic variant thereof) described herein) or a nucleic acid molecule (e.g., an RNA molecule, e.g., an mRNA molecule) described herein (e.g., a nucleic acid molecule (e.g., an RNA molecule, e.g., an mRNA molecule) comprising a coding region encoding a SARS-CoV-2 spike protein or polypeptide (e.g., a SARS-CoV-2 spike protein or polypeptide immunogen (or immunogenic fragment and/or immunogenic variant thereof) described herein), (e) a vaccine composition described herein (e.g., a vaccine composition comprising any of the foregoing), or (f) a pharmaceutical composition described herein (e.g., a pharmaceutical composition comprising any of the foregoing), to thereby induce and/or enhance an immune response in the subject.

In some embodiments, the SARS-CoV-2 spike protein or polypeptide (e.g., the SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition is administered to the subject in an amount and for a time sufficient to induce and/or enhance an immune response the subject.

An immune response in a subject can be measured by common methods known to those of skill in the art. For example, serological assays can be employed to detect a humoral response by measuring titers of anti-antigen (e.g., anti-SARS-CoV-2 spike protein, anti-SARS-CoV-2 spike protein RBD) IgG antibodies post administration. For example, an enzyme-linked immunosorbent assay (ELISA) is a standard laboratory test for detecting and quantifying antibodies well known to the person of skill in the art. Generally, blood is collected from a consenting subject, centrifuged, and the serum isolated according to standard techniques. The recombinant target antigen (e.g., SARS-CoV-2 spike protein, SARS-CoV-2 spike protein RBD) is immobilized in microplate wells. The microplate is blocked by through the incubation with an irrelevant antigen (e.g., bovine serum albumin). The serum sample from the subject is prepared and added to the blocked wells to allow for binding of an antigen specific antibodies to the immobilized antigen. The bound antibodies are detected using a secondary tagged antibody that binds to the previously bound antibodies (e.g., anti-human IgG antibodies). See, e.g., Front. Immunol., Forgacs David et al., SARS-CoV-2 mRNA Vaccines Elicit Different Responses in Immunologically Naïve and Pre-Immune Humans; Vol 12 (27 Sep. 2021) https://doi.org/10.3389/fimmu.2021.728021, the entire contents of which is incorporated by reference herein for all purposes.

Cell based assays can also be utilized to detect a cell based immune response (e.g., T cell immune response). For example, antigen specific T cells (e.g., CD4+ or CD8+ T cells) can be measured using an enzyme-linked immunospot (ELISpot), an intracellular cytokine staining (ICS) assay, or an activation induced marker assay (AIM). Each of these assays is commonly used to detect cell based (e.g., T cell) immune responses to vaccines and well known to the person of ordinary skill in the art. See, e.g., Bowyer, Georgina et al. “Activation-induced Markers Detect Vaccine-Specific CD4+ T Cell Responses Not Measured by Assays Conventionally Used in Clinical Trials.” Vaccines vol. 6, 3 50. 31 Jul. 2018, doi:10.3390/vaccines6030050, the entire contents of which is incorporated by reference herein for all purposes.

5.10.3 Methods of Preventing, Ameliorating, and/or Treating a SARS-CoV-2 Infection

In one aspect, provided herein are methods of preventing, ameliorating, and/or treating a SARS-CoV-2 infection in a subject in need thereof, the method comprising administering to the subject (a) a SARS-CoV-2 spike protein or polypeptide described herein (e.g., a SARS-CoV-2 spike protein or polypeptide immunogen (or immunogenic fragment and/or immunogenic variant thereof) described herein) (or a fusion or conjugate thereof), (b) a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein) (or a fusion or conjugate thereof), (c) a vector described herein (e.g., a vector comprising a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein), (d) a carrier described herein (e.g., a carrier comprising a SARS-CoV-2 spike protein or polypeptide described herein (e.g., a SARS-CoV-2 spike protein or polypeptide immunogen (or immunogenic fragment and/or immunogenic variant thereof) described herein) or a nucleic acid molecule (e.g., an RNA molecule, e.g., an mRNA molecule) described herein (e.g., a nucleic acid molecule (e.g., an RNA molecule, e.g., an mRNA molecule) comprising a coding region encoding a SARS-CoV-2 spike protein or polypeptide (e.g., a SARS-CoV-2 spike protein or polypeptide immunogen (or immunogenic fragment and/or immunogenic variant thereof) described herein), (e) a vaccine composition described herein (e.g., a vaccine composition comprising any of the foregoing), or (f) a pharmaceutical composition described herein (e.g., a pharmaceutical composition comprising any of the foregoing), to thereby prevent, ameliorate, and/or treat the SARS-CoV-2 infection in the subject.

In some embodiments, the SARS-CoV-2 spike protein or polypeptide (e.g., the SARS-CoV-2 protein or peptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition is administered to the subject in an amount and for a time sufficient to prevent, ameliorate, and/or treat the SARS-CoV-2 infection the subject.

In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, or the pharmaceutical composition is administered to the subject as a prophylactic treatment. In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, or the pharmaceutical composition is administered as a treatment after the onset of at least one symptom of a SARS-CoV-2 infection or a SARS-CoV-2 infection associated disease.

In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition is administered to the subject after a determination that the subject does or does not have a SARS-CoV-2 infection.

In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition prevents infection with SARS-CoV-2, reduces the likelihood of infection with SARS-CoV-2, reduces the likelihood of developing an established infection after challenge with SARS-CoV-2, reduces the duration of a SARS-CoV-2 infection, prevents or delays onset one or more symptoms of COVID-19, reduces the frequency and/or severity one or more symptoms of COVID-19, and/or reduces the risk of hospitalization or death associated with COVID-19, or any combination of thereof.

Exemplary COVID-19 symptoms include, but are not limited to, shortness of breath, difficulty breathing, respiratory rate greater than or equal to 20 breaths per minutes, abnormal SpO2, clinical or radiological evidence of lower respiratory tract disease, radiological evidence of deep vein thrombosis, respiratory failure, evidence of shock, significant renal, hepatic, and neurological dysfunction.

The SARS-CoV-2 spike proteins (e.g., immunogens (or immunogenic fragments or immunogenic variants thereof)), the nucleic acid molecules (e.g., RNA molecules, e.g., mRNA molecules), the vectors, the carriers, the vaccine compositions, and the pharmaceutical compositions described herein may be administered as a prime and/or a boost in a homologous or heterologous prime-boost regimen.

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is administered to the subject as a prime in a homologous or heterologous prime-boost regimen.

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is administered to the subject as a boost in a homologous or heterologous prime-boost regimen.

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is administered to the subject as a vaccine prime and a vaccine boost in a homologous prime-boost regimen.

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is administered to the subject as a prime in a heterologous prime-boost regimen. The boost vaccine composition in the regimen may be a vaccine that is based on mRNAs, DNAs, viral vectors (e.g., adenoviral vectors, adeno-associated viral vectors, lentiviral vectors, vesicular stomatitis viral vectors, vaccinia viral vectors, or measles viral vectors), peptides or proteins, viral-like particles (VLP), capsid-like particles (CLP), live attenuated viruses, inactivated viruses (killed vaccines), and the like. In some embodiments, the prime vaccine composition contains the same immunogen as the booster vaccine. In some embodiments, the primary vaccine contains a different immunogen as the booster vaccine.

In some embodiments, the SARS-CoV-2 spike proteins (e.g., immunogens (or immunogenic fragments or immunogenic variants thereof)), the nucleic acid molecules (e.g., RNA molecules, e.g., mRNA molecules), the vectors, the carriers, the vaccine compositions, and the pharmaceutical compositions described herein are administered to a subject as a boost in a heterologous prime-boost regimen. The prime vaccine composition in the regimen may be a vaccine that is based on mRNAs, DNAs, viral vectors (e.g., adenoviral vectors, adeno-associated viral vectors, lentiviral vectors, vesicular stomatitis viral vectors, vaccinia viral vectors, or measles viral vectors), peptides or proteins, viral-like particles (VLP), capsid-like particles (CLP), live attenuated viruses, inactivated viruses (killed vaccines), and the like. In some embodiments, the prime vaccine composition contains the same immunogen as the booster vaccine. In some embodiments, the primary vaccine contains a different immunogen as the booster vaccine.

In some embodiments, a single dose of the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition is administered to the subject. In some embodiments, a series of doses of the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition are administered to the subject (e.g., two doses given at a set interval (e.g., 2 weeks, 3 weeks apart) or within a range (e.g., 2-6 weeks apart)).

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition is administered to the subject in a therapeutically effective amount. In some embodiments, wherein an mRNA molecule encoding a SARS-CoV-2 spike protein (e.g., a SARS-CoV-2 spike protein or polypeptide immunogen (or immunogenic fragment and/or immunogenic variant thereof)) described herein (e.g., a vaccine or pharmaceutical composition comprising the same) is administered to the subject, the mRNA molecule is administered at a dose from about 30-200 mcg (e.g., 30 mcg, 50 mcg, 75 mcg, 100 mcg, 150 mcg, or 200 mcg).

### 5.10.4 Methods of Vaccination Against SARS-CoV-2

Provided herein are, inter alia, various methods of vaccinating subjects (e.g., human subjects) against SARS-CoV-2. As such, provided below in §§ 5.10.4.1 and 5.10.4.2, are various methods of vaccinating subjects utilizing one or more of the SARS-CoV-2 spike proteins (e.g., immunogens (or immunogenic fragments or immunogenic variants thereof)), the nucleic acid molecules (e.g., RNA molecules, e.g., mRNA molecules), the vectors, the carriers, the vaccine compositions, and the pharmaceutical compositions described herein.

### 5.10.4.1 Methods of Vaccinating a Subject Against SARS-CoV-2

In one aspect, provided herein are methods of vaccinating a subject against SARS-CoV-2, the method comprising administering to the subject to the subject (a) a SARS-CoV-2 spike protein or polypeptide described herein (e.g., a SARS-CoV-2 spike protein or polypeptide immunogen (or immunogenic fragment and/or immunogenic variant thereof) described herein) (or a fusion or conjugate thereof), (b) a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein) (or a fusion or conjugate thereof), (c) a vector described herein (e.g., a vector comprising a nucleic acid molecule described herein (e.g., a nucleic acid molecule (e.g., an RNA molecule, e.g., an mRNA molecule) comprising a coding region encoding a SARS-CoV-2 spike protein (e.g., a SARS-CoV-2 spike protein or polypeptide immunogen (or immunogenic fragment and/or immunogenic variant thereof) described herein), (d) a carrier described herein (e.g., a carrier comprising a SARS-CoV-2 spike protein or polypeptide described herein (e.g., a SARS-CoV-2 spike protein or polypeptide immunogen (or immunogenic fragment and/or immunogenic variant thereof) described herein) or a nucleic acid molecule (e.g., an RNA molecule, e.g., an mRNA molecule) described herein (e.g., a nucleic acid molecule (e.g., an RNA molecule, e.g., an mRNA molecule) comprising a coding region encoding a SARS-CoV-2 spike protein or polypeptide (e.g., a SARS-CoV-2 spike protein or polypeptide immunogen (or immunogenic fragment and/or immunogenic variant thereof) described herein), (e) a vaccine composition described herein (e.g., a vaccine composition comprising any of the foregoing), or (f) a pharmaceutical composition described herein (e.g., a pharmaceutical composition comprising any of the foregoing), to thereby vaccinate the subject against SARS-CoV-2.

In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition is administered to the subject in an amount and for a time sufficient to vaccinate the subject against SARS-CoV-2.

In some embodiments, the SARS-CoV-2 spike proteins (e.g., immunogens (or immunogenic fragments or immunogenic variants thereof)), the nucleic acid molecules (e.g., RNA molecules, e.g., mRNA molecules), the vectors, the carriers, the vaccine compositions, and the pharmaceutical compositions described herein are administered as a prime and/or a boost in a homologous or heterologous prime-boost regimen.

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is administered to the subject as a prime in a homologous or heterologous prime-boost regimen.

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is administered to the subject as a boost in a homologous or heterologous prime-boost regimen.

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is administered to the subject as a vaccine prime and a vaccine boost in a homologous prime-boost regimen.

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is administered to the subject as a prime in a heterologous prime-boost regimen. The boost vaccine composition in the regimen may be a vaccine that is based on mRNAs, DNAs, viral vectors (e.g., adenoviral vectors, adeno-associated viral vectors, lentiviral vectors, vesicular stomatitis viral vectors, vaccinia viral vectors, or measles viral vectors), peptides or proteins, viral-like particles (VLP), capsid-like particles (CLP), live attenuated viruses, inactivated viruses (killed vaccines), and the like. In some embodiments, the prime vaccine composition contains the same immunogen as the booster vaccine. In some embodiments, the primary vaccine contains a different immunogen as the booster vaccine.

In some embodiments, the SARS-CoV-2 spike proteins (e.g., immunogens (or immunogenic fragments or immunogenic variants thereof)), the nucleic acid molecules (e.g., RNA molecules, e.g., mRNA molecules), the vectors, the carriers, the vaccine compositions, and the pharmaceutical compositions described herein are administered to a subject as a boost in a heterologous prime-boost regimen. The prime vaccine composition in the regimen may be a vaccine that is based on mRNAs, DNAs, viral vectors (e.g., adenoviral vectors, adeno-associated viral vectors, lentiviral vectors, vesicular stomatitis viral vectors, vaccinia viral vectors, or measles viral vectors), peptides or proteins, viral-like particles (VLP), capsid-like particles (CLP), live attenuated viruses, inactivated viruses (killed vaccines), and the like. In some embodiments, the prime vaccine composition contains the same immunogen as the booster vaccine. In some embodiments, the primary vaccine contains a different immunogen as the booster vaccine.

In some embodiments, a single dose of the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition is administered to the subject. In some embodiments, a series of doses of the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition are administered to the subject (e.g., two doses given at a set interval (e.g., 2 weeks, 3 weeks apart) or within a range (e.g., 2-6 weeks apart)).

In some embodiments, the SARS-CoV-2 spike protein (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition is administered to the subject in a therapeutically effective amount. In some embodiments, wherein an mRNA molecule encoding a SARS-CoV-2 spike protein (e.g., a SARS-CoV-2 spike protein or polypeptide immunogen (or immunogenic fragment and/or immunogenic variant thereof)) described herein (e.g., a vaccine or pharmaceutical composition comprising the same) is administered to the subject, the mRNA molecule is administered at a dose from about 30-200 mcg (e.g., 30 mcg, 50 mcg, 75 mcg, 100 mcg, 150 mcg, or 200 mcg).

In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, or the pharmaceutical composition is administered to the subject as a prophylactic treatment. In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, or the pharmaceutical composition is administered as a treatment after the onset of at least one symptom of a SARS-CoV-2 infection or a SARS-CoV-2 infection associated disease.

In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition is administered to the subject after a determination that the subject does or does not have a SARS-CoV-2 infection.

In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., the RNA molecule, e.g., the mRNA molecule), the vector, the carrier, the vaccine composition, or the pharmaceutical composition prevents infection with SARS-CoV-2, reduces the likelihood of infection with SARS-CoV-2, reduces the likelihood of developing an established infection after challenge with SARS-CoV-2, reduces the duration of a SARS-CoV-2 infection, prevents or delays onset one or more symptoms of COVID-19, reduces the frequency and/or severity one or more symptoms of COVID-19, and/or reduces the risk of hospitalization or death associated with COVID-19, or any combination of thereof.

Exemplary COVID-19 symptoms include, but are not limited to, shortness of breath, difficulty breathing, respiratory rate greater than or equal to 20 breaths per minutes, abnormal SpO2, clinical or radiological evidence of lower respiratory tract disease, radiological evidence of deep vein thrombosis, respiratory failure, evidence of shock, significant renal, hepatic, and neurological dysfunction.

5.10.4.2 Methods of Vaccinating a Subject Utilizing a SARS-CoV-2 mRNA Vaccine

In one aspect, provided herein are methods of vaccinating in a subject against SARS-CoV-2, the method comprising administering to the subject to the subject (a) an mRNA molecule (e.g., an mRNA molecule described herein) encoding a SARS-CoV-2 spike protein (e.g., a SARS-CoV-2 spike protein or polypeptide immunogen (or immunogenic fragment and/or immunogenic variant thereof)) described herein (or a fusion or conjugate thereof), (b) a vector comprising the mRNA molecule, (c) a carrier comprising the mRNA molecule or the vector, (d) a vaccine composition comprising the mRNA molecule, the vector, or the carrier, or (e) a pharmaceutical composition comprising the mRNA molecule, the vector, the carrier, or the vaccine composition, to thereby vaccinate the subject against SARS-CoV-2.

In some embodiments, the mRNA molecule is formulated in a lipid nanoparticle, the vaccine composition having the following characteristics: (a) the LNPs comprise a cationic lipid, a neutral lipid, a cholesterol, and a PEG lipid, (b) the LNPs have a mean particle size of between 80 nm and 160 nm, and (c) the mRNA comprises: (i) a 5′-cap structure; (ii) a 5′-UTR; (iii) N1-methyl-pseudouridine, cytosine, adenine, and guanine; (iv) a 3′-UTR; and (v) a poly-A region

In some embodiments, the SARS-CoV-2 spike protein or polypeptide (e.g., a SARS-CoV-2 spike protein or polypeptide immunogen (or immunogenic fragment and/or immunogenic variant thereof)), the mRNA molecule, the vector, the carrier, the vaccine composition, or the pharmaceutical composition is administered to the subject in an amount and for a time sufficient to vaccinate the subject against SARS-CoV-2.

In some embodiments, the mRNA molecules, the vectors, the carriers, the vaccine compositions, and the pharmaceutical compositions described herein are administered as a prime and/or a boost in a homologous or heterologous prime-boost regimen.

In some embodiments, the mRNA molecule, the vector, the carrier, the vaccine composition, or the pharmaceutical composition described herein is administered to the subject as a prime in a homologous or heterologous prime-boost regimen.

In some embodiments, the mRNA molecule, the vector, the carrier, the vaccine composition, or the pharmaceutical composition described herein is administered to the subject as a boost in a homologous or heterologous prime-boost regimen.

In some embodiments, the mRNA molecule, the vector, the carrier, the vaccine composition, or the pharmaceutical composition described herein is administered to the subject as a vaccine prime and a vaccine boost in a homologous prime-boost regimen.

In some embodiments, the mRNA molecule, the vector, the carrier, the vaccine composition, or the pharmaceutical composition described herein is administered to the subject as a prime in a heterologous prime-boost regimen. The boost vaccine composition in the regimen may be a vaccine that is based on mRNAs, DNAs, viral vectors (e.g., adenoviral vectors, adeno-associated viral vectors, lentiviral vectors, vesicular stomatitis viral vectors, vaccinia viral vectors, or measles viral vectors), peptides or proteins, viral-like particles (VLP), capsid-like particles (CLP), live attenuated viruses, inactivated viruses (killed vaccines), and the like. In some embodiments, the prime vaccine composition contains the same immunogen as the booster vaccine. In some embodiments, the primary vaccine contains a different immunogen as the booster vaccine.

In some embodiments, the mRNA molecules, the vectors, the carriers, the vaccine compositions, and the pharmaceutical compositions described herein are administered to a subject as a boost in a heterologous prime-boost regimen. The prime vaccine composition in the regimen may be a vaccine that is based on mRNAs, DNAs, viral vectors (e.g., adenoviral vectors, adeno-associated viral vectors, lentiviral vectors, vesicular stomatitis viral vectors, vaccinia viral vectors, or measles viral vectors), peptides or proteins, viral-like particles (VLP), capsid-like particles (CLP), live attenuated viruses, inactivated viruses (killed vaccines), and the like. In some embodiments, the prime vaccine composition contains the same immunogen as the booster vaccine. In some embodiments, the primary vaccine contains a different immunogen as the booster vaccine.

In some embodiments, a single dose of the mRNA molecule, the vector, the carrier, the vaccine composition, or the pharmaceutical composition is administered to the subject. In some embodiments, a series of doses of the mRNA molecule, the vector, the carrier, the vaccine composition, or the pharmaceutical composition are administered to the subject (e.g., two doses given at a set interval (e.g., 2 weeks, 3 weeks apart) or within a range (e.g., 2-6 weeks apart)).

In some embodiments, the mRNA molecule, the vector, the carrier, the vaccine composition, or the pharmaceutical composition is administered to the subject in a therapeutically effective amount. In some embodiments, the mRNA molecule is administered at a dose from about 30-200 mcg (e.g., 30 mcg, 50 mcg, 75 mcg, 100 mcg, 150 mcg, or 200 mcg).

In some embodiments, the mRNA molecule, the vector, the carrier, or the pharmaceutical composition is administered to the subject as a prophylactic treatment. In some embodiments, the mRNA molecule, the vector, the carrier, or the pharmaceutical composition is administered as a treatment after the onset of at least one symptom of a SARS-CoV-2 infection or a SARS-CoV-2 infection associated disease.

In some embodiments, the mRNA molecule, the vector, the carrier, the vaccine composition, or the pharmaceutical composition is administered to the subject after a determination that the subject does or does not have a SARS-CoV-2 infection.

In some embodiments, the mRNA molecule, the vector, the carrier, the vaccine composition, or the pharmaceutical composition prevents infection with SARS-CoV-2, reduces the likelihood of infection with SARS-CoV-2, reduces the likelihood of developing an established infection after challenge with SARS-CoV-2, reduces the duration of a SARS-CoV-2 infection, prevents or delays onset one or more symptoms of COVID-19, reduces the frequency and/or severity one or more symptoms of COVID-19, and/or reduces the risk of hospitalization or death associated with COVID-19, or any combination of thereof.

Exemplary COVID-19 symptoms include, but are not limited to, shortness of breath, difficulty breathing, respiratory rate greater than or equal to 20 breaths per minutes, abnormal SpO2, clinical or radiological evidence of lower respiratory tract disease, radiological evidence of deep vein thrombosis, respiratory failure, evidence of shock, significant renal, hepatic, and neurological dysfunction.

### 5.11 Kits

In a one aspect, provided herein are kits comprising at least one SARS-CoV-2 spike protein or polypeptide (e.g., a SARS-CoV-2 spike protein or polypeptide immunogen (or immunogenic fragment and/or immunogenic variant thereof)) described herein, a nucleic acid molecule (e.g., RNA molecule, e.g., mRNA molecule) described herein, a vector described herein, a carrier described herein, a vaccine composition described herein, and/or a pharmaceutical composition described herein. In addition, the kit may comprise a liquid vehicle for solubilizing or diluting any one of the foregoing, and/or technical instructions. The technical instructions of the kit may contain information about administration and dosage and subjects (e.g., subject groups).

In some embodiments, the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is provided in a separate part of the kit, wherein the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein is optionally lyophilized, spray-dried, or spray-freeze dried. The kit may further contain as a part a vehicle (e.g., buffer solution) for solubilizing the dried or lyophilized pharmaceutical composition.

In some embodiments, the kit comprises a single dose container of the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition. In some embodiments, the kit comprises a multi-dose container for administration of the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein and/or an administration device (e.g., an injector for intradermal injection or a syringe for intramuscular injection).

In some embodiments, the kit comprises an adjuvant in a separate container from the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein. The kit may further contain technical instructions for mixing the SARS-CoV-2 spike protein or polypeptide (e.g., a SARS-CoV-2 spike protein or polypeptide immunogen (or immunogenic fragment and/or immunogenic variant thereof)), the nucleic acid molecule (e.g., RNA molecule, e.g., mRNA molecule), the vector, the carrier, the vaccine composition, or the pharmaceutical composition described herein and the adjuvant prior to administration or for co-administration.

Any of the kits described herein may be used in any of the methods described herein, e.g., in § 5.10. Any of the kits described herein may be used in a treatment or prophylaxis as defined herein (e.g., for the treatment, amelioration, and/or prophylaxis of SARS-CoV-2 infection).

### 6. EXAMPLES

### 6.1 Example 1. SARS-CoV-2 RNA Vaccines

The following example provides an exemplary method of preparing an mRNA vaccine comprising an mRNA encoding any one or a plurality of the immunogens identified herein (e.g., an immunogen comprising an amino acid substitution set forth in Table 2).

### 6.1.1 DNA Preparation

DNA constructs comprising SARS-CoV-2 proteins (e.g., immunogens) comprising at least one of the amino acid substitutions set forth in Table 2 are prepared and used for subsequent RNA in vitro transcription. Preparation of the DNA coding sequences can include codon optimization for stabilization and expression by introducing specific codons to generate a DNA coding sequence with an optimized G/C content (as discussed herein). Optimized coding sequences are introduced into a DNA plasmid comprising a 3′-UTR, a 5-UTR, and polyadenylation sequence. The obtained DNA plasmids are transformed and propagated in bacteria using common protocols known in the art. The DNA plasmids are subsequently extracted, purified, and used for RNA in vitro transcription.

### 6.1.2 RNA In Vitro Transcription

The DNA plasmids are enzymatically linearized using a restriction enzyme used for DNA dependent RNA in vitro transcription using T7 RNA polymerase in the presence of a nucleotide mixture (ATP/GTP/CTP/UTP) and a 5′ cap (or analog) under suitable buffer conditions. The obtained RNA constructs are purified using a suitable method known in the art e.g., RP-HPLC. The RNA in vitro transcription is performed in the presence of modified nucleotides for incorporation in the RNA e.g., pseudouridine or N1-methylpseudouridine (m1ψ) instead of UTP. In some instances, the 5′ cap is enzymatically added to the RNA after in vitro transcription using capping enzymes as commonly known in the art.

### 6.1.3 Preparation of LNPs and Encapsulated RNAs

LNPs are prepared using according to the general procedures known in the art using e.g., cationic lipids, structural lipids, a PEG-lipids, and cholesterol see, e.g., WO2015199952, WO2017004143 and WO2017075531, the full contents of each of which is incorporated by reference herein for al purposes. The lipid solution (in ethanol) is mixed with RNA (in aqueous solution) in a small T-piece to encapsulate the mRNA in the LNPs, see, e.g., Reichmuth, Andreas M et al. “mRNA vaccine delivery using lipid nanoparticles.” Therapeutic delivery vol. 7, 5 (2016): 319-34. doi:10.4155/tde-2016-0006, the entire contents of which is incorporated by reference herein for all purposes. The LNP formulated mRNA is rebuffered as needed via dialysis and concentrated to a target concentration using ultracentrifugation.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

What is claimed is:

1. A nucleic acid molecule comprising a coding region encoding a SARS-CoV-2 spike protein (e.g., a SARS-CoV-2 spike protein immunogen (or an immunogenic fragment and/or immunogenic variant thereof)), wherein the amino acid sequence of the SARS-CoV-2 spike protein (e.g., the SARS-CoV-2 spike protein immunogen (or immunogenic fragment and/or immunogenic variant thereof)) comprises at least one set of amino acid substitutions set forth in Table 2.

2. The nucleic acid molecule of claim 1, wherein the amino acid sequence of the encoded SARS-CoV-2 spike protein (e.g., the SARS-CoV-2 spike protein immunogen (or immunogenic fragment and/or immunogenic variant thereof)) comprises or consists of the SARS-CoV-2 spike protein receptor binding domain (RBD).

3. The nucleic acid molecule of claim 1 or 2, wherein the amino acid sequence of the encoded SARS-CoV-2 spike protein (e.g., the SARS-CoV-2 spike protein immunogen (or immunogenic fragment and/or immunogenic variant thereof)) comprises or consists of a full-length SARS-CoV-2 spike protein.

4. The nucleic acid molecule of any one of the preceding claims, wherein the amino acid sequence of the encoded SARS-CoV-2 spike protein (e.g., the SARS-CoV-2 spike protein immunogen (or immunogenic fragment and/or immunogenic variant thereof)) comprises at least 10, 15, 20, 30, 40, 50, 60, 70, 80, 90, 100, 150, 200, 300, 400, 500, 600, 700, 800, 900, 1000, 1100, 1200, or 1300 amino acids.

5. The nucleic acid molecule of any one of the preceding claims, wherein the amino acid sequence of the encoded SARS-CoV-2 spike protein (e.g., the SARS-CoV-2 spike protein immunogen (or immunogenic fragment and/or immunogenic variant thereof)) comprises from about 10-15, 10-20, 10-30, 10-40, 10-50, 10-60, 10-70, 10-80, 10-90, 10-100, 10-200, 10-300, 10-400, 10-500, 10-600, 10-700, 10-800, 10-900, 10-1000, 10-1100, 10-1200, or 10-1300 amino acids.

6. The nucleic acid molecule of any one of the preceding claims, wherein other than the at least one set of amino acid substitutions set forth in Table 2, the amino acid sequence of the encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, 1200, or 1300 amino acids set forth in any one of SEQ ID NOS: 1-4.

7. The nucleic acid molecule of any one of the preceding claims, wherein other than the at least one set of amino acid substitutions set forth in Table 2, the amino acid sequence of the encoded SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-4.

8. The nucleic acid molecule of any one of the preceding claims, wherein the amino acid sequence of the encoded SARS-CoV-2 spike protein (e.g., the SARS-CoV-2 spike protein immunogen (or immunogenic fragment and/or immunogenic variant thereof)) comprises a plurality of sets of amino acid substitutions set forth in Table 2.

9. The nucleic acid molecule of any one of the preceding claims, wherein the amino acid sequence of the encoded SARS-CoV-2 spike protein (e.g., the SARS-CoV-2 spike protein immunogen (or immunogenic fragment and/or immunogenic variant thereof)) comprises at least 2, 3, 4, 5, or 6 or more sets of amino acid substitutions set forth in Table 2.

10. The nucleic acid molecule of any one of the preceding claims, wherein the amino acid sequence of the encoded SARS-CoV-2 spike protein (e.g., the SARS-CoV-2 spike protein immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%) amino acid variations (e.g., substitutions, additions, deletions, etc.) that are not set forth in Table 2.

11. The nucleic acid molecule of any one of the preceding claims, wherein the amino acid sequence of the encoded SARS-CoV-2 spike protein (e.g., the SARS-CoV-2 spike protein immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%) amino acid variations (e.g., substitutions, additions, deletions, etc.) that are not set forth in Table 2 relative to the amino acid sequence set forth in any one of SEQ ID NOS: 1-4.

12. The nucleic acid molecule of any one of the preceding claims, wherein the encoded SARS-CoV-2 spike protein (e.g., the SARS-CoV-2 spike protein immunogen (or immunogenic fragment and/or immunogenic variant thereof)) is stabilized in a prefusion state.

13. The nucleic acid molecule of any one of the preceding claims, wherein the amino acid sequence of the encoded SARS-CoV-2 spike protein (e.g., the SARS-CoV-2 spike protein immunogen (or immunogenic fragment and/or immunogenic variant thereof)) comprises at least one amino acid variation relative to the amino acid sequence set forth in any one of SEQ ID NOS: 1-4, that stabilizes the SARS-CoV-2 spike protein (e.g., the SARS-CoV-2 spike protein immunogen (or immunogenic fragment and/or immunogenic variant thereof)) in a prefusion state.

14. The nucleic acid molecule of any one of the preceding claims, wherein the amino acid sequence of the encoded SARS-CoV-2 spike protein (e.g., the SARS-CoV-2 spike protein immunogen (or immunogenic fragment and/or immunogenic variant thereof)) comprises a proline at amino acid position 986 and/or a proline at amino acid position 987, amino acid numbering relative to the amino acid positions set forth in SEQ ID NO: 4.

15. The nucleic acid molecule of any one of the preceding claims, wherein the encoded SARS-CoV-2 spike protein (e.g., the SARS-CoV-2 spike protein immunogen (or immunogenic fragment and/or immunogenic variant thereof)) comprises one or more non-naturally N-glycosylation sites.

16. The nucleic acid molecule of any one of the preceding claims, wherein the amino acid sequence of the encoded SARS-CoV-2 spike protein (e.g., the SARS-CoV-2 spike protein immunogen (or immunogenic fragment and/or immunogenic variant thereof)) comprises the addition of one or more N-glycosylation sites relative to the amino acid sequence set forth in any one of SEQ ID NOS: 1-4.

17. The nucleic acid molecule of any one of the preceding claims, wherein the encoded SARS-CoV-2 spike protein is an immunogen (or an immunogenic fragment and/or immunogenic variant thereof).

18. The nucleic acid molecule of any one of the preceding claims, wherein the nucleic acid molecule is RNA or DNA.

19. The nucleic acid molecule of any one of the preceding claims, wherein the RNA is messenger ribonucleic acid (mRNA).

20. The nucleic acid molecule of any one of the preceding claims, wherein the nucleic acid molecule comprises at least one modified nucleotide.

21. The nucleic acid molecule of any one of the preceding claims, wherein the nucleic acid molecule comprises N1-methyl-pseudouridine, cytosine, adenine, and guanine.

22. The nucleic acid molecule of any one of the preceding claims, wherein the nucleic acid molecule comprises a heterologous 5′-untranslated region (UTR), 3′-UTR, or both a 5′-UTR and 3′-UTR.

23. The nucleic acid molecule of any one of the preceding claims, wherein the nucleic acid molecule comprises a poly(A) sequence.

24. The nucleic acid molecule of any one of the preceding claims, wherein the nucleic acid molecule comprises a 5′cap structure.

25. The nucleic acid molecule of any one of the preceding claims, wherein the nucleotide sequence of the nucleic acid molecule is codon optimized.

26. The nucleic acid molecule of any one of the preceding claims, wherein the nucleic acid molecule further encodes a heterologous polypeptide or protein.

27. The nucleic acid molecule of any one of the preceding claims, wherein the nucleic acid molecule encodes a signal peptide.

28. The nucleic acid molecule of any one of the preceding claims, wherein the nucleic acid molecule encodes a homologous or heterologous signal peptide.

29. The nucleic acid molecule of any one of the preceding claims, wherein the nucleic acid molecule does not encode a signal peptide.

30. A vector comprising the nucleic acid molecule of any one of the preceding claims.

31. The vector of claim 30, wherein the vector is a non-viral vector (e.g., a plasmid) or a viral vector.

32. A conjugate comprising the nucleic acid molecule of any one of claims 1-29 operably connected (e.g., directly or indirectly (e.g., via a linker)) to a heterologous moiety (e.g., a heterologous polypeptide or protein).

33. A composition comprising at least one of the nucleic acid molecules set forth in any one of claims 1-29.

34. The composition of claim 33, comprising a plurality of nucleic acid molecules set forth in any one of claims 1-29, wherein the amino acid sequence of each of the encoded SARS-CoV-2 spike proteins (e.g., SARS-CoV-2 spike protein immunogens (or immunogenic fragments and/or immunogenic variants thereof)) encoded by of each of the plurality of nucleic acid molecules comprises a different set of amino acid substitutions set forth in Table 2.

35. The composition of claim 33 or 34, wherein the composition comprises a nucleic acid molecule comprising a coding region encoding SARS-CoV-2 spike proteins (e.g., SARS-CoV-2 spike protein immunogens (or immunogenic fragments and/or immunogenic variants thereof)) comprising an amino acid sequence that does not comprise a set of amino acid substitutions set forth in Table 2.

36. A SARS-CoV-2 spike protein (e.g., a SARS-CoV-2 spike protein immunogen (or an immunogenic fragment and/or immunogenic variant thereof)) wherein the amino acid sequence of the SARS-CoV-2 spike protein (e.g., the SARS-CoV-2 spike protein immunogen (or immunogenic fragment and/or immunogenic variant thereof)) comprises at least one set of amino acid substitutions set forth in Table 2.

37. The SARS-CoV-2 spike protein (e.g., the SARS-CoV-2 spike protein immunogen (or immunogenic fragment and/or immunogenic variant thereof)) of claim 36, wherein the amino acid sequence of the SARS-CoV-2 spike protein (e.g., the SARS-CoV-2 spike protein immunogen (or immunogenic fragment and/or immunogenic variant thereof)) comprises or consists of the receptor binding domain (RBD) of a SARS-CoV-2 spike protein.

38. The SARS-CoV-2 spike protein (e.g., the SARS-CoV-2 spike protein immunogen (or immunogenic fragment and/or immunogenic variant thereof)) of claim 36 or 37, wherein the amino acid sequence of the SARS-CoV-2 spike protein (e.g., the SARS-CoV-2 spike protein immunogen (or immunogenic fragment and/or immunogenic variant thereof)) comprises or consists of a full-length SARS-CoV-2 spike protein.

39. The SARS-CoV-2 spike protein (e.g., the SARS-CoV-2 spike protein immunogen (or immunogenic fragment and/or immunogenic variant thereof)) of any one of claims 36-38, wherein the amino acid sequence of the SARS-CoV-2 spike protein (e.g., the SARS-CoV-2 spike protein immunogen (or immunogenic fragment and/or immunogenic variant thereof)) comprises at least 10, 15, 20, 30, 40, 50, 60, 70, 80, 90, 100, 150, 200, 300, 400, 500, 600, 700, 800, 900, 1000, 1100, or 1200 amino acids.

40. The SARS-CoV-2 spike protein (e.g., the SARS-CoV-2 spike protein immunogen (or immunogenic fragment and/or immunogenic variant thereof)) of any one of claims 36-39, wherein the amino acid sequence of the SARS-CoV-2 spike protein (e.g., the SARS-CoV-2 spike protein immunogen (or immunogenic fragment and/or immunogenic variant thereof)) comprises from about 10-15, 10-20, 10-30, 10-40, 10-50, 10-60, 10-70, 10-80, 10-90, 10-100, 10-200, 10-300, 10-400, 10-500, 10-600, 10-700, 10-800, 10-900, 10-1000, 10-1100, 10-1200, or 10-1300 amino acids.

41. The SARS-CoV-2 spike protein (e.g., the SARS-CoV-2 spike protein immunogen (or immunogenic fragment and/or immunogenic variant thereof)) of any one of claims 36-40, wherein other than the at least one set of amino acid substitutions, the amino acid sequence of the SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to a contiguous stretch of at least 10, 15, 20, 30, 40, 50, 60, 70, 80, 90, 100, 150, 200, 300, 400, 500, 600, 700, 800, 900, 1000, 1100, or 1200 amino acids set forth in any one of SEQ ID NOS: 1-4.

42. The SARS-CoV-2 spike protein (e.g., the SARS-CoV-2 spike protein immunogen (or immunogenic fragment and/or immunogenic variant thereof)) of any one of claims 36-41, wherein other than the at least one set of amino acid substitutions, the amino acid sequence of the SARS-CoV-2 spike protein (e.g., the SARS-CoV-2 spike protein immunogen (or immunogenic fragment and/or immunogenic variant thereof)) is at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in any one of SEQ ID NOS: 1-4.

43. The SARS-CoV-2 spike protein (e.g., the SARS-CoV-2 spike protein immunogen (or immunogenic fragment and/or immunogenic variant thereof)) of any one of claims 36-42, wherein the amino acid sequence of the SARS-CoV-2 spike protein (e.g., the SARS-CoV-2 spike protein immunogen (or immunogenic fragment and/or immunogenic variant thereof)) comprises a plurality of amino acid substitutions set forth in Table 2.

44. The SARS-CoV-2 spike protein (e.g., the SARS-CoV-2 spike protein immunogen (or immunogenic fragment and/or immunogenic variant thereof)) of any one of claims 36-43, wherein the amino acid sequence of the SARS-CoV-2 spike protein (e.g., the SARS-CoV-2 spike protein immunogen (or immunogenic fragment and/or immunogenic variant thereof)) comprises at least 2, 3, 4, 5, or 6 or more sets of amino acid substitutions set forth in Table 2.

45. The SARS-CoV-2 spike protein (e.g., the SARS-CoV-2 spike protein immunogen (or immunogenic fragment and/or immunogenic variant thereof)) of any one of claims 36-44, wherein the amino acid sequence of the SARS-CoV-2 spike protein (e.g., the SARS-CoV-2 spike protein immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%) amino acid variations (e.g., substitutions, additions, deletions, etc.) that are not listed in Table 2.

46. The SARS-CoV-2 spike protein (e.g., the SARS-CoV-2 spike protein immunogen (or immunogenic fragment and/or immunogenic variant thereof)) of any one of claims 36-45, wherein the amino acid sequence of the SARS-CoV-2 spike protein (e.g., the SARS-CoV-2 spike protein immunogen (or immunogenic fragment and/or immunogenic variant thereof)) comprises 1 or more (e.g., 1, 2, 3, 4, 5, 6, 7, 8, 9, or 10, or more) but less than 20% (e.g., less than 15%, less than 12%, less than 10%, less than 8%, less than 5%) amino acid variations (e.g., substitutions, additions, deletions, etc.) that are not listed in Table 2 relative to the amino acid sequence set forth in any one of SEQ ID NOS: 1-4.

47. The SARS-CoV-2 spike protein (e.g., the SARS-CoV-2 spike protein immunogen (or immunogenic fragment and/or immunogenic variant thereof)) of any one of claims 36-46, wherein the SARS-CoV-2 spike protein (e.g., the SARS-CoV-2 spike protein immunogen (or immunogenic fragment and/or immunogenic variant thereof)) is stabilized in a prefusion state.

48. The SARS-CoV-2 spike protein (e.g., the SARS-CoV-2 spike protein immunogen (or immunogenic fragment and/or immunogenic variant thereof)) of any one of claims 36-47, wherein the amino acid sequence of the SARS-CoV-2 spike protein (e.g., the SARS-CoV-2 spike protein immunogen (or immunogenic fragment and/or immunogenic variant thereof)) comprises at least one amino acid variation relative to the amino acid sequence set forth in any one of SEQ ID NOS: 1-4 that stabilizes the SARS-CoV-2 spike protein (e.g., the SARS-CoV-2 spike protein immunogen (or immunogenic fragment and/or immunogenic variant thereof)) in a prefusion state.

49. The SARS-CoV-2 spike protein (e.g., the SARS-CoV-2 spike protein immunogen (or immunogenic fragment and/or immunogenic variant thereof)) of any one of claims 36-48, wherein the amino acid sequence of the SARS-CoV-2 spike protein (e.g., the SARS-CoV-2 spike protein immunogen (or immunogenic fragment and/or immunogenic variant thereof)) comprises a proline at amino acid position 986 and/or a proline at amino acid position 987, amino acid numbering relative to the amino acid positions set forth in SEQ ID NO: 4.

50. The SARS-CoV-2 spike protein (e.g., the SARS-CoV-2 spike protein immunogen (or immunogenic fragment and/or immunogenic variant thereof)) of any one of claims 36-49, wherein the SARS-CoV-2 spike protein (e.g., the SARS-CoV-2 spike protein immunogen (or immunogenic fragment and/or immunogenic variant thereof)) comprises one or more non-naturally N-glycosylation sites.

51. The SARS-CoV-2 spike protein (e.g., the SARS-CoV-2 spike protein immunogen (or immunogenic fragment and/or immunogenic variant thereof)) of any one of claims 36-50, wherein the amino acid sequence of the SARS-CoV-2 spike protein (e.g., the SARS-CoV-2 spike protein immunogen (or immunogenic fragment and/or immunogenic variant thereof)) comprises the addition of one or more N-glycosylation sites relative to the amino acid sequence set forth in any one of SEQ ID NOS: 1-4.

52. The SARS-CoV-2 spike protein (e.g., the SARS-CoV-2 spike protein immunogen (or immunogenic fragment and/or immunogenic variant thereof)) of any one of claims 36-51, wherein the amino acid sequence of the SARS-CoV-2 spike protein (e.g., the SARS-CoV-2 spike protein immunogen (or immunogenic fragment and/or immunogenic variant thereof)) comprises an inactive furin cleavage site.

53. The SARS-CoV-2 spike protein (e.g., the SARS-CoV-2 spike protein immunogen (or immunogenic fragment and/or immunogenic variant thereof)) of any one of claims 36-52, wherein the amino acid sequence of the SARS-CoV-2 spike protein (e.g., the SARS-CoV-2 spike protein immunogen (or immunogenic fragment and/or immunogenic variant thereof)) comprises at least one amino acid variation in the furin cleavage site that inactivates the furin cleavage site.

54. The SARS-CoV-2 spike protein (e.g., the SARS-CoV-2 spike protein immunogen (or immunogenic fragment and/or immunogenic variant thereof)) of any one of claims 36-53, wherein the SARS-CoV-2 spike protein (e.g., the SARS-CoV-2 spike protein immunogen (or immunogenic fragment and/or immunogenic variant thereof)) further comprises a heterologous protein.

55. The SARS-CoV-2 spike protein (e.g., the SARS-CoV-2 spike protein immunogen (or immunogenic fragment and/or immunogenic variant thereof)) of any one of claims 36-54, wherein the SARS-CoV-2 spike protein (e.g., the SARS-CoV-2 spike protein immunogen (or immunogenic fragment and/or immunogenic variant thereof)) comprises a signal peptide.

56. The SARS-CoV-2 spike protein (e.g., the SARS-CoV-2 spike protein immunogen (or immunogenic fragment and/or immunogenic variant thereof)) of any one of claims 36-55, wherein the SARS-CoV-2 spike protein (e.g., the SARS-CoV-2 spike protein immunogen (or immunogenic fragment and/or immunogenic variant thereof)) comprises a homologous or heterologous signal peptide.

57. The SARS-CoV-2 spike protein (e.g., the SARS-CoV-2 spike protein immunogen (or immunogenic fragment and/or immunogenic variant thereof)) of any one of claims 36-56, wherein the SARS-CoV-2 spike protein (e.g., the SARS-CoV-2 spike protein immunogen (or immunogenic fragment and/or immunogenic variant thereof)) does not comprise a signal peptide.

58. The SARS-CoV-2 spike protein (e.g., the SARS-CoV-2 spike protein immunogen (or immunogenic fragment and/or immunogenic variant thereof)) of any one of claims 36-57, wherein the SARS-CoV-2 spike protein is an immunogen (or an immunogenic fragment and/or immunogenic variant thereof).

59. A composition comprising at least one SARS-CoV-2 spike proteins (e.g., at least one of the SARS-CoV-2 spike protein immunogens (or immunogenic fragments and/or immunogenic variants thereof)) set forth in any one of claims 36-58.

60. The composition of claim 59, wherein the composition comprises a plurality of SARS-CoV-2 spike proteins (e.g., a plurality of SARS-CoV-2 spike protein immunogens (or immunogenic fragments and/or immunogenic variants thereof)) set forth in any one of claims 36-58, wherein the amino acid sequence of each of the plurality of SARS-CoV-2 spike proteins (e.g., the SARS-CoV-2 spike protein immunogens (or immunogenic fragments and/or immunogenic variants thereof)) comprises a different set of amino acid substitutions set forth in Table 2.

61. The composition of claim 59 or 60, wherein the composition comprises at least one SARS-CoV-2 spike protein (e.g., SARS-CoV-2 spike protein immunogen (or immunogenic fragment and/or immunogenic variant thereof)) comprising an amino acid sequence that does not comprise a set of amino acid substitutions set forth in Table 2.

62. A fusion protein comprising the SARS-CoV-2 spike protein (e.g., the SARS-CoV-2 spike protein immunogen (or immunogenic fragment and/or immunogenic variant thereof)) of any one of claims 36-58 operably connected (e.g., directly or indirectly (e.g., via a linker)) to a heterologous polypeptide or protein.

63. A conjugate comprising the SARS-CoV-2 spike protein (e.g., the SARS-CoV-2 spike protein immunogen (or immunogenic fragment and/or immunogenic variant thereof)) of any one of claims 36-58 operably connected (e.g., directly or indirectly (e.g., via a linker)) to a heterologous moiety.

64. A SARS-CoV-2 spike protein (e.g., a SARS-CoV-2 spike protein immunogen (or an immunogenic fragment and/or immunogenic variant thereof)) encoded by the nucleic acid molecule of any one of claims 1-29.

65. A nucleic acid molecule encoding the SARS-CoV-2 spike protein (e.g., the SARS-CoV-2 spike protein immunogen (or immunogenic fragment and/or immunogenic variant thereof)) of any one of claims 36-58.

66. A carrier comprising the nucleic acid molecule of any one of claims 1-29 or 65, the vector of any one of claims 30-31, the composition of any one of claims 33-35 or 59-61, the SARS-CoV-2 spike protein (e.g., the SARS-CoV-2 spike protein immunogen (or immunogenic fragment and/or immunogenic variant thereof)) of any one of claims 36-58 or 64, the conjugate of any one of claim 32 or 63, the fusion protein of claim 62, the vaccine composition of any one of claims 71-77, or the pharmaceutical composition of any one of claims 78-80.

67. The carrier of claim 66, wherein the carrier is a lipid nanoparticle (LNP), liposome, lipoplex, or nanoliposome.

68. The carrier of claim 67, wherein the carrier is an LNP.

69. The carrier of claim 67 or 68, wherein the LNP comprises a cationic lipid, a neutral lipid, a cholesterol, and/or a PEG lipid.

70. The carrier of any one of claims 67-69, wherein the LNP has a mean particle size of between 80 nm and 160 nm.

71. A vaccine composition comprising the nucleic acid molecule of any one of claims 1-29 or 65, the vector of any one of claims 30-31, the composition of any one of claims 33-35 or 59-61, the SARS-CoV-2 spike protein (e.g., the SARS-CoV-2 spike protein immunogen (or immunogenic fragment and/or immunogenic variant thereof)) of any one of claims 36-58 or 64, the vaccine composition of any one of claims 71-77, the conjugate of any one of claim 32 or 63, the fusion protein of claim 62, the carrier of any one of claims 66-70, or the pharmaceutical composition of any one of claims 78-80.

72. The vaccine composition of claim 71, wherein the vaccine composition is a prime vaccine composition.

73. The vaccine composition of claim 71 or 72, wherein the vaccine composition is a boost vaccine composition.

74. The vaccine composition of any one of claims 71-73, wherein the vaccine composition is a prime vaccine composition and a boost vaccine composition.

75. The vaccine composition of any one of claims 71-74, wherein the vaccine composition can be utilized as a prime vaccine composition and/or a booster vaccine composition in a homologous or heterologous prime boost vaccine regimen.

76. A vaccine composition comprising a messenger ribonucleic acid (mRNA) encoding a SARS-CoV-2 spike protein (e.g., a SARS-CoV-2 spike protein immunogen (or an immunogenic fragment and/or immunogenic variant thereof)) that comprises at least one amino acid substitution set forth in Table 2, formulated in a lipid nanoparticle, the vaccine composition having the following characteristics: (a) the LNPs comprise a cationic lipid, a neutral lipid, a cholesterol, and a PEG lipid, (b) the LNPs have a mean particle size of between 80 nm and 160 nm, and (c) the mRNA comprises: (i) a 5′-cap structure; (ii) a 5′-UTR; (iii) N1-methyl-pseudouridine, cytosine, adenine, and guanine; (iv) a 3′-UTR; and (v) a poly-A region.

77. The vaccine composition of any one of claims 71-76, wherein the vaccine composition further comprises an adjuvant.

78. A pharmaceutical composition comprising the nucleic acid molecule of any one of claims 1-29 or 65, the vector of any one of claims 30-31, the composition of any one of claims 33-35 or 59-61, the SARS-CoV-2 spike protein (e.g., the SARS-CoV-2 spike protein immunogen (or immunogenic fragment and/or immunogenic variant thereof)) of any one of claims 36-58 or 64, the vaccine composition of any one of claims 71-77, the conjugate of any one of claim 32 or 63, the fusion protein of claim 62, the carrier of any one of claims 66-70, and a pharmaceutically acceptable excipient.

79. A pharmaceutical composition comprising a messenger ribonucleic acid (mRNA) encoding a SARS-CoV-2 spike protein (e.g., a SARS-CoV-2 spike protein immunogen (or an immunogenic fragment and/or immunogenic variant thereof)) that comprises at least one amino acid substitution set forth in Table 2, formulated in a lipid nanoparticle, the pharmaceutical composition having the following characteristics: (a) the LNPs comprise a cationic lipid, a neutral lipid, a cholesterol, and a PEG lipid, (b) the LNPs have a mean particle size of between 80 nm and 160 nm, and (c) the mRNA comprises: (i) a 5′-cap structure; (ii) a 5′-UTR; (iii) N1-methyl-pseudouridine, cytosine, adenine, and guanine; (iv) a 3′-UTR; and (v) a poly-A region.

80. The method of any one of claims 78-79, wherein the pharmaceutical composition further comprises an adjuvant.

81. A kit comprising the nucleic acid molecule of any one of claims 1-29 or 65, the vector of any one of claims 30-31, the composition of any one of claims 33-35 or 59-61, the SARS-CoV-2 spike protein (e.g., the SARS-CoV-2 spike protein immunogen (or immunogenic fragment and/or immunogenic variant thereof)) of any one of claims 36-58 or 64, the vaccine composition of any one of claims 71-77, the conjugate of any one of claim 32 or 63, the fusion protein of claim 62, the carrier of any one of claims 66-70, or the pharmaceutical composition of any one of claims 78-80.

82. The kit of claim 81, comprising instructions for use of the nucleic acid molecule, vector, protein (or immunogenic fragment or immunogenic variant thereof), conjugate, fusion protein, carrier, composition, vaccine composition, or pharmaceutical composition.

83. A method of delivering a nucleic acid molecule, vector, protein (or immunogenic fragment or immunogenic variant thereof), conjugate, fusion protein, carrier, composition, vaccine composition, or pharmaceutical composition to a subject in need thereof, the method comprising administering to the subject the nucleic acid molecule of any one of claims 1-29 or 65, the vector of any one of claims 30-31, the SARS-CoV-2 spike protein (e.g., the SARS-CoV-2 spike protein immunogen (or immunogenic fragment and/or immunogenic variant thereof)) of any one of claims 36-58 or 64, the conjugate of claim 32 or 63, the fusion protein of claim 62, the carrier of any one of claims 66-70, the composition of any one of claims 33-35 or 59-61, the vaccine composition of any one of claims 71-77, or the pharmaceutical composition of any one of claims 78-80, to thereby deliver the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition to the subject.

84. A method of inducing or enhancing an immune response in a subject in need thereof, the method comprising administering to the subject the nucleic acid molecule of any one of claims 1-29 or 65, the vector of any one of claims 30-31, the SARS-CoV-2 spike protein (e.g., the SARS-CoV-2 spike protein immunogen (or immunogenic fragment and/or immunogenic variant thereof)) of any one of claims 36-58 or 64, the conjugate of claim 32 or 63, the fusion protein of claim 62, the carrier of any one of claims 66-70, the composition of any one of claims 33-35 or 59-61, the vaccine composition of any one of claims 71-77, or the pharmaceutical composition of any one of claims 78-80, to thereby induce or enhance an immune response the subject.

85. A method of preventing, ameliorating, or treating a SARS-CoV-2 infection in a subject in need thereof, the method comprising administering to the subject the nucleic acid molecule of any one of claims 1-29 or 65, the vector of any one of claims 30-31, the SARS-CoV-2 spike protein (e.g., the SARS-CoV-2 spike protein immunogen (or immunogenic fragment and/or immunogenic variant thereof)) of any one of claims 36-58 or 64, the conjugate of claim 32 or 63, the fusion protein of claim 62, the carrier of any one of claims 66-70, the composition of any one of claims 33-35 or 59-61, the vaccine composition of any one of claims 71-77, or the pharmaceutical composition of any one of claims 78-80, to thereby prevent, ameliorate, or treat the SARS-CoV-2 infection the subject.

86. A method of vaccinating a subject against SARS-CoV-2, the method comprising administering to the subject the nucleic acid molecule of any one of claims 1-29 or 65, the vector of any one of claims 30-31, the SARS-CoV-2 spike protein (e.g., the SARS-CoV-2 spike protein immunogen (or immunogenic fragment and/or immunogenic variant thereof)) of any one of claims 36-58 or 64, the conjugate of claim 32 or 63, the fusion protein of claim 62, the carrier of any one of claims 66-70, the composition of any one of claims 33-35 or 59-61, the vaccine composition of any one of claims 71-77, or the pharmaceutical composition of any one of claims 78-80, to thereby vaccinate the subject against SARS-CoV-2.

87. A method of vaccinating a subject against SARS-CoV-2, the method comprising administering to the subject (a) an mRNA molecule (e.g., an mRNA molecule described herein) encoding the SARS-CoV-2 spike protein (e.g., a SARS-CoV-2 spike protein or polypeptide immunogen (or immunogenic fragment and/or immunogenic variant thereof)) of any one of claims 36-58 or 64 (or a conjugate or fusion protein thereof), (b) a vector comprising the mRNA molecule, (c) a carrier comprising the mRNA molecule or the vector, (d) a vaccine composition comprising the mRNA molecule, the vector, or the carrier, or (e) a pharmaceutical composition comprising the mRNA molecule, the vector, the carrier, or the vaccine composition, to thereby vaccinate the subject against SARS-CoV-2, to thereby vaccinate the subject against SARS-CoV-2.

88. A method of vaccinating a subject against SARS-CoV-2, the method comprising administering to the subject the vaccine composition of any one of claims 76-77 or the pharmaceutical composition of any one of claims 78-80, to thereby vaccinate the subject against SARS-CoV-2.

89. The method of any one of claims 83-88, wherein the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered to the subject at least twice.

90. The method of any one of claims 83-89, wherein the subject is a human.

91. The method of any one of claims 83-90, wherein the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a prime and/or a boost in a homologous or heterologous prime-boost regimen.

92. The method of any one of claims 83-91, wherein the nucleic acid molecule, the vector, the SARS-CoV-2 spike protein (e.g., the SARS-CoV-2 spike protein immunogen (or immunogenic fragment and/or immunogenic variant thereof)), the conjugate, the fusion protein, the carrier, the composition, the vaccine composition, or the pharmaceutical composition is administered as a boost in a heterologous prime-boost regimen.


SEQUENCE LISTING (7 sequences)

SEQ ID NO: 1 - protein, 201 aa, Severe acute respiratory symdrome coronavirus 2
NITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKST

SEQ ID NO: 2 - protein, 1270 aa, Severe acute respiratory symdrome coronavirus 2
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVEPERDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1195 aa, Severe acute respiratory symdrome coronavirus 2
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVEPERDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIK

SEQ ID NO: 4 - protein, 1273 aa, Severe acute respiratory symdrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 4 aa, Severe acute respiratory symdrome coronavirus 2
RRAR

SEQ ID NO: 6 - protein, 4 aa, synthetic construct
QQAQ

SEQ ID NO: 7 - RNA, 500 nt, synthetic construct
  source 1..500: mol_type = other RNA
  misc_difference 1..500: note = This sequence may encompass 10, 20, 30, 40, 50, 60, 70, 80, 90, 100, 200, 300, 400, or 500 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa